SEQUENCE LISTING (32 sequences)

SEQ ID NO: 1 - protein, 472 aa, Cucurbita moschata
  PriL isoform 1 Protein
MELHRSQRKSSTATISSTTLPLYRSAPPLEVRLEEFELYAIDRLRVLKGISDGLSRGKKSEDMEKLVRDLLKAHMKHPQASEAVNKDIISHFVLRLVYCRTEDLRKWFLSMETMLFRHRFLSESPESQKQVFSELGLSYKAIGYAEFEAVKDKLVQVARLIGQPVPSADAIYYKVPWEEVPELVAGRRVLLHKGYAYIAIYQVVSLVATQFRSYLSKALSLTNRKWTSTIREQEKDRLTPIVEALCTSYLGPDYSQPTEYADISIKDLEQIAKSSFPLCMRHLFDKLKEDHHLKHGGRMQLGLFLKGVGLKLDDALAFWRAEFSQRVGVERFDKEYAYSIRHNYGKEGKRVDYSPYSCQKVISSSPGVGDHHGCPYRHFSEDNLRAALGKMGVNNQKMEDILDKVRNRHYQLACTLTFESVHGSSCDAGINHPNQYFIDSQKVLQSKVDPETSSCTNFDAFLTVLSLGRLST

SEQ ID NO: 2 - DNA, 1419 bp, Cucurbita moschata
  PriL isoform 1 CDS
atggaactccatcgttctcagaggaaatcttccaccgcgaccatttcctcgaccactcttccactctatcgctccgctcctcctctcgaagtcaggcttgaagaattcgaactttatgccatagatcgtcttcgagttcttaaaggaatttctgatggtttatctcgaggaaagaaatctgaagacatggagaaactggttagagacttgttgaaggcccatatgaaacatccacaggcatccgaggctgtgaacaaggatataatatctcactttgttctgcgcctcgtatactgcagaacggaggacttgagaaaatggtttctttctatggaaactatgctatttcgacaccgttttctttctgaaagtcctgaatctcagaagcaggtcttttcggagcttggcctctcatacaaagcaatcggttatgcagagtttgaggctgtgaaggacaaattggtccaagttgctcggttgattggtcagcctgtaccaagcgctgatgcaatatactataaggtaccatgggaagaagttccagaactggtggctggtcgaagggtattacttcataaaggatatgcatatattgctatctatcaggtggtttcccttgttgcaacacaattccgcagttacctatcaaaggccttaagtctgacaaacaggaaatggacatctacaataagagaacaagagaaggatcggttgacccctatcgttgaagccctttgcactagctacctgggtcctgactactcacagccaacagagtatgctgatatatcaatcaaagaccttgaacaaattgctaaaagttcatttcctctttgcatgcggcacctatttgataagctgaaagaagatcatcatttgaagcatggagggagaatgcaattaggtctctttcttaagggtgttggtttgaagcttgatgatgcccttgctttctggagagctgagttctcgcagagagttggtgttgagaggtttgacaaagaatatgcatacagtatcaggcataactatggaaaagaaggcaagagagtggattattcaccttattcctgtcaaaaagtcatctcatcatcacctggtgttggagatcatcatggatgtccctatagacatttcagtgaagacaacttaagagcagctcttggtaaaatgggagtaaataaccagaaaatggaagatatattggacaaagtgcgaaatagacactatcagttggcctgcaccttgacatttgaatcggttcatggttcgtcatgcgacgccgggattaatcatccaaaccagtacttcattgatagtcaaaaagttttgcaatctaaggtagaccctgaaacttcatcatgtactaattttgatgctttcttgacagttttgagtcttggaaggctgagcacataa

SEQ ID NO: 3 - protein, 452 aa, Cucurbita moschata
  PriL isoform 2 Protein
MELHRSQRKSSTATISSTTLPLYRSAPPLEVRLEEFELYAIDRLRVLKGISDGLSRGKKSEDMEKLVRDLLKAHMKHPQASEAVNKDIISHFVLRLVYCRTEDLRKWFLSMETMLFRHRFLSESPESQKQVFSELGLSYKAIGYAEFEAVKDKLVQVARLIGQPVPSADAIYYKVPWEEVPELVAGRRVLLHKGYAYIAIYQVVSLVATQFRSYLSKALSLTNRKWTSTIREQEKDRLTPIVEALCTSYLGPDYSQPTEYADISIKDLEQIAKSSFPLCMRHLFDKLKEDHHLKHGGRMQLGLFLKGVGLKLDDALAFWRAEFSQRVGVERFDKEYAYSIRHNYGKEGKRVDYSPYSCQKVISSSPGVGDHHGCPYRHFSEDNLRAALGKMGVNNQKMEDILDKVRNRHYQLACTLTFESVHGSSCDAGINHPNQYFIDSQKVLQSKNNSTA

SEQ ID NO: 4 - DNA, 1359 bp, Cucurbita moschata
  PriL isoform 2 CDS
atggaactccatcgttctcagaggaaatcttccaccgcgaccatttcctcgaccactcttccactctatcgctccgctcctcctctcgaagtcaggcttgaagaattcgaactttatgccatagatcgtcttcgagttcttaaaggaatttctgatggtttatctcgaggaaagaaatctgaagacatggagaaactggttagagacttgttgaaggcccatatgaaacatccacaggcatccgaggctgtgaacaaggatataatatctcactttgttctgcgcctcgtatactgcagaacggaggacttgagaaaatggtttctttctatggaaactatgctatttcgacaccgttttctttctgaaagtcctgaatctcagaagcaggtcttttcggagcttggcctctcatacaaagcaatcggttatgcagagtttgaggctgtgaaggacaaattggtccaagttgctcggttgattggtcagcctgtaccaagcgctgatgcaatatactataaggtaccatgggaagaagttccagaactggtggctggtcgaagggtattacttcataaaggatatgcatatattgctatctatcaggtggtttcccttgttgcaacacaattccgcagttacctatcaaaggccttaagtctgacaaacaggaaatggacatctacaataagagaacaagagaaggatcggttgacccctatcgttgaagccctttgcactagctacctgggtcctgactactcacagccaacagagtatgctgatatatcaatcaaagaccttgaacaaattgctaaaagttcatttcctctttgcatgcggcacctatttgataagctgaaagaagatcatcatttgaagcatggagggagaatgcaattaggtctctttcttaagggtgttggtttgaagcttgatgatgcccttgctttctggagagctgagttctcgcagagagttggtgttgagaggtttgacaaagaatatgcatacagtatcaggcataactatggaaaagaaggcaagagagtggattattcaccttattcctgtcaaaaagtcatctcatcatcacctggtgttggagatcatcatggatgtccctatagacatttcagtgaagacaacttaagagcagctcttggtaaaatgggagtaaataaccagaaaatggaagatatattggacaaagtgcgaaatagacactatcagttggcctgcaccttgacatttgaatcggttcatggttcgtcatgcgacgccgggattaatcatccaaaccagtacttcattgatagtcaaaaagttttgcaatctaagaacaattcaacagcttag

SEQ ID NO: 5 - protein, 452 aa, Cucurbita moschata
  Modified PriL Protein
MELHRSQRKSSTATTSSTTLPLYRSAPPLEVRLEEFELYAIDRLRVLKGISDGLSRGKKSEEMEKLVRDLLKAHMKHPQASEAVNKDIISHFVLRLVYCRTEDLRKWFLSMETMLFRHRFLSKGPESQKQVFSELGLSYKAISYAECEAVKDKLVQVARLIDHPVPSADAIYYKVPWEEVPELVAGRRVLLHKGYAYIAIYQVVSLVATQFRSYLSKALSLTNRKWTSTIREQEKDRLTPIVEALCTSYLGPDYSQPTEYADISIKDLEQIAKSSFPLCMRHLFDKLKEDHHLKHGGRMQLGLFLKGVGLKLDDALAFWRAEFSQRVGVERFDKEYAYSIRHNYGKEGKRVDYSPYSCQKVISSSPGVGDHHGCPYRHFSEDNLRAALGKMGVNNQKMEDILDKVRNRHYQLACTLTFESVHGSSCDAGINHPNQYFIDSQKVLQSKNNSTA

SEQ ID NO: 6 - DNA, 1359 bp, Cucurbita moschata
  Modified PriL CDS
atggaactccatcgttctcagaggaaatcttccaccgccaccacttcctcgaccactcttccactctatcgctccgctcctcctctcgaagtcaggcttgaagaattcgaactttatgccatagatcgtcttcgagttcttaaaggaatttctgatggtttatctcgaggaaagaaatctgaagaaatggagaaactggttagagacttgttgaaggcccatatgaaacatccacaggcatccgaggctgtgaacaaggatataatatctcactttgttctgcgcctcgtatactgcagaacggaggacttgagaaaatggtttctttctatggaaactatgctatttcgacaccgttttctttctaaaggtcctgaatctcagaagcaggtcttttcggagcttggcctctcatacaaagcaatcagttatgcagagtgtgaggctgtgaaggacaaattggtccaagttgctcgattgattgatcaccctgtaccaagcgctgatgcaatatactataaggtaccatgggaagaagttccagaactggtggctggtcgaagggtattacttcataaaggatatgcatatattgctatctatcaggtggtttcccttgttgcaacacaattccgcagttacctatcaaaggccttaagtctgacaaacaggaaatggacatctacaataagagaacaagagaaggatcggttgacccctatcgttgaagccctttgcacaagctacctgggtcctgactactcacagccaacggagtatgctgatatatcaatcaaagaccttgaacaaattgctaaaagttcatttcctctttgcatgcggcacctatttgataagctgaaagaagatcatcatttgaagcatggagggagaatgcaattaggtctctttctcaagggtgttggtttgaagcttgatgatgcccttgctttctggagagctgagttctcgcagagagttggtgttgagaggtttgacaaagaatatgcatacagtatcaggcataactatggaaaagaaggcaagagagtggattattcaccttattcctgtcaaaaagtcatctcatcatcacctggtgttggagatcatcatggatgtccctatagacatttcagtgaagacaacttaagagcagctcttggtaaaatgggagtaaataaccagaaaatggaagatatattggacaaagtgcgaaatagacattatcagttggcctgcaccttgacgtttgaatcggttcatggttcatcgtgcgacgctgggattaatcatccaaaccagtacttcattgatagtcaaaaagttctgcaatctaagaacaattcaacagcttag

SEQ ID NO: 7 - protein, 426 aa, Cucumis melo
  PriL Protein
MEPYLPQRKSSVSTNSTTALPLYRSAPPLEVRLEDFELYAIDRLRVLKGISDGLSRGKKSEEMEKLVRELLKTNMKHPQASEVVNKDIISHFVLRLVYCRTEDLRKWFLSMETMLFRHRFLSESPESQKQVFAELGLSYKAISNAEFEAVRDKLVQVARLIGQPAPSSDAIYYKVPWEEVPELVAGRRVFLHKGYAYIAIYQVVSLVATQFRSYLSKALSLTNRKWTSTIREQEKDRLTPIVEALCTSYLGPDYSQPREYADISIKDLDQIAKSSFPLCMRHLFEKLKEDHHLKHGGRMQLGLFLKGVGLKLDDALAFWRAEFSQRVGAERFDKEYAYSIRHNYGKEGKRVDYSPYSCQKIISSSPSVGDHHGCPYRHFSEDNLRAALGKMGVNNRTMDDIMDKVRNRHYQVLNLLPLNFYVVSWE

SEQ ID NO: 8 - DNA, 1281 bp, Cucumis melo
  PriL CDS
atggaaccctatcttcctcagagaaaatcttccgtctcgaccaattccactaccgctctcccactctaccgctctgctcctcctcttgaagtcaggcttgaagattttgagctttatgccatagatcgtcttcgagttcttaaagggatttctgatgggctatctcgaggaaagaaatctgaagaaatggagaaactggttagagaattgttgaaaaccaacatgaaacatccccaggcatctgaggttgtgaacaaggatataatatctcactttgttctgcgccttgtgtattgccgaacggaggacttgagaaaatggtttctttctatggaaactatgctattccgacaccgttttctttctgaaagtcctgaatctcagaagcaggtatttgcggagcttggtctctcatacaaagcaatcagtaatgcagaatttgaggctgtaagggacaaattggttcaagttgctcggttgattggtcagcctgcaccaagtagtgatgctatatactataaggtaccatgggaagaagttccagagcttgtggctggtcgaagagtatttctccataaaggatatgcatatattgctatttatcaggtggtttcccttgttgcaacacaattccgcagttacctgtcaaaggccctaagtctgacgaacaggaaatggacatctacaataagagaacaagagaaagatcggttgaccccaatagtagaagccctttgcacgagctacctgggtcctgactactcacagccaagagagtatgctgatatatcaataaaagaccttgaccaaatagctaaaagttcatttcctctttgcatgcgacacctatttgaaaagctgaaagaagatcatcatttgaagcatggagggaggatgcaattaggtctctttctcaagggtgttggtttgaagcttgatgatgctctggctttctggagagctgagttctcccagagagttggtgctgagaggtttgacaaagaatatgcatacagtatcaggcataactatggaaaagaaggcaagagagtggattattcgccttattcctgtcaaaaaataatctcatcatcacctagtgttggagatcatcatggatgtccctatagacatttcagtgaagacaacttaagagcagctcttggtaaaatgggagtaaataaccggacaatggatgatataatggacaaagtgcgaaatagacattatcaggttctcaatttactgcctcttaatttttatgttgtcagttgggaataa

SEQ ID NO: 9 - protein, 452 aa, Cucumis melo
  Modified PriL Protein
MEPHLPQRKSSVSTNSTTALPLYRSAPPLEVRLEDFELYAIDRLRVLKGISDGLSRGKKSEEMEKLVKELLKTNMKHPQASEVVNKDIISHFVLRLVYCRTEDLRKWFLSMETMLFRHRFLSGSLESQKQVFAELGLSYKAISNAEFEAVRDKLVQVARLIGQPAQSSDAIYYKVPWEEVPELVAGRRVFLHKGYAYIATYQVVSLVATQFRSYLSKALSLTNRKWTSTIREQEKDRLTPIVEALCTSYLGPDYSQPREYADISIKDLDQIAKSSFPLCMRHLFEKLKEDHHLKHGGRMQLGLFLKGVGLKLDDALAFWRAEFSQRVGAERFDKEYAYSIRHNYGKEGKRVDYSPYSCQKIISSSPSVGDHHGCPYRHFSEDNLRAALGKMGVNNRTMDDIMDKVRNRHYQLACTLTFESIHGSTCDAGINHPNQYFIDSQKVLQSKNNSTS

SEQ ID NO: 10 - DNA, 1359 bp, Cucumis melo
  Modified PriL CDS
atggaaccccatcttcctcagagaaaatcttccgtctcgaccaattccactaccgctctcccactctaccgctctgctcctcctcttgaagtcaggcttgaagattttgagctttatgccatagatcgtcttcgagttcttaaagggatttctgatgggctatctcgaggaaagaaatctgaagaaatggagaaactggttaaagaattgttgaaaaccaacatgaaacatccccaggcatctgaggttgtgaacaaggatataatatctcactttgttctgcgccttgtgtattgccgaacggaggacttgagaaaatggtttctttctatggaaactatgctattccgacaccgttttctttctggaagtcttgaatctcagaagcaggtatttgcggagcttggtctctcatacaaagcaatcagtaatgcagaatttgaggctgtaagggacaaattggttcaagttgctcggttgattggtcagcctgcacaaagtagtgatgctatatactataaggtaccatgggaagaagttccagagcttgtggctggtcgaagagtatttctccataaaggatatgcatatattgctacttatcaggtggtttcccttgttgcaacacaattccgcagttacctgtcaaaggccctaagtctgacgaacaggaaatggacatctacaataagagaacaagagaaagatcggttgaccccaatagtagaagccctttgcacgagctacctgggtcctgactactcacagccaagagagtatgctgatatatcaataaaagaccttgaccaaatagctaaaagttcatttcctctttgcatgcgacacctatttgaaaagctgaaagaagatcatcatttgaagcatggagggaggatgcaattaggtctctttctcaagggtgttggtttgaagcttgatgatgctctggctttctggagagctgagttctcccagagagttggtgctgagaggtttgacaaagaatatgcatacagtatcaggcataactatggaaaagaaggcaagagagtggattattcgccttattcctgtcaaaaaataatctcatcatcacctagtgttggagatcatcatggatgtccctatagacatttcagtgaagacaacttaagagcagctcttggtaaaatgggagtaaataaccggacaatggatgatataatggacaaagtgcgaaatagacattatcagttggcatgcaccttgacatttgaatcgatccatggctcgacatgtgatgctgggattaatcatccaaaccagtacttcattgatagtcaaaaggtcctgcaatctaagaataattcaacatcctag

SEQ ID NO: 11 - protein, 381 aa, Cucurbita pepo subsp. pepo
  PriL Protein
MELHRSQRKSSTATTSSTTLPLYRSAPPLEVRLEEFELYAIDRLRVLKGISDGLSRGKKSEEMEKLVRDLLKAHMKHPQASEAVNKDIISHFVLRLVYCRTEDLRKWFLSMETMLFRHRFLSESPESQKQVFSELGLSYKAISYAEFEAVKDKLVQVARLIGQPVPSADAIYYKVVSLVATQFRSYLSKALSLTNRKWTSTIREQEKDRLTPIVEALCTSYLGPDYSQPTEYADISIKDLEQIAKSSFPLCMRHLFDKLKEDHHLKHGGRMQLGLFLKGVGLKLDDALAFWRAEFSQRVGVERFDKEYAYSIRHNYGKEGKRVLACTLTFESVHGSSCDAGINHPNQYFIDSQKVLQSKHWENALARRVGSNQVLSLDREL

SEQ ID NO: 12 - DNA, 1146 bp, Cucurbita pepo subsp. pepo
  PriL CDS
atggaactccatcgttctcagaggaaatcttccaccgccaccacttcctcgaccactcttccactctatcgctcagctcctcctctcgaagtcaggcttgaagaattcgaactttatgccatagatcgtcttcgagttcttaaaggaatttctgatggtttatctcgaggaaaaaaatctgaagaaatggagaaactggtaagagacttgttgaaggcccatatgaaacatccacaggcatccgaggctgtgaacaaggatataatatctcactttgttctgcgcctcgtatactgcagaacggaggacttgagaaaatggtttctttctatggaaactatgctatttcgacaccgttttctttctgaaagtcctgaatctcagaagcaggtcttttcggagcttggcctctcatacaaagcaatcagttatgcagagtttgaggctgtgaaggacaaattggtccaagttgctcggttgattggtcagcctgtaccaagcgctgatgcaatatactataaggtggtttcccttgttgcaacacaattccgcagttacctatcaaaggccttaagtctgacaaacaggaaatggacatctacaataagagaacaagagaaggatcggttgacccctatcgttgaagccctttgcacaagctacctgggtcctgactactcacagccaacagagtatgctgatatatcaatcaaagaccttgaacaaattgctaaaagttcatttcctctttgcatgcggcacctatttgataagctgaaagaagatcatcatttgaagcatggagggagaatgcaattaggtctctttctcaagggtgttggtttgaagcttgatgatgcccttgctttctggagagctgagttctcgcagagagttggtgttgagaggtttgacaaagaatatgcatacagtatcaggcataactatggaaaagaaggcaagagagtgttggcatgcaccttgacgtttgaatcggttcatggttcatcgtgcgacgctgggattaatcatccaaaccagtacttcattgatagtcaaaaagttctgcaatctaagcactgggaaaacgctcttgcaaggcgagtgggttcaaaccaggtgctttctctcgatcgagagctttaa

SEQ ID NO: 13 - protein, 452 aa, Cucurbita maxima
  PriL Protein
MELHRSQRKSSTATTSSTTLPLYRSAPPLEVRLEEFELYAIDRLRVLKGISDGLSRGKKSEEMEKLVRGLLNAHMKHPQASEAVNKDIISHFVLRLVYCRTEDLRKWFLSMETMLFRHRFLSESPESQKQVFSELGLSYKAISYAEFEAVKDKLVQVARLIGQPVPSADAIYYKVPWEEVPELVAGRRVLLHKGYAYIAIYQVVSLVATQFRSYLSKTLSLTNRKWTSTIREQEKDRLTPIVEALCTSYLGPDYSQPTEYADISIKDLEQIAKSSFPLCMRHLFDKLKEDHHLKHGGRMQLGLFLKGVGLKLDDALTFWRAEFSQRVGVERFDKEYAYSIRHNYGKEGKRVDYSPYSCQKIISSSPGVGDHHGCPYRHFSEDNLRAALGKMGVNNQKIEDILDKVRNRHYQLACTLTFESVHGSSCDAGINHPNQYFIDSQKVLQSKNNSTA

SEQ ID NO: 14 - DNA, 1359 bp, Cucurbita maxima
  PriL CDS
atggaactccatcgttctcagaggaaatcttccaccgccaccacttcctcgaccactcttccactctatcgctccgctcctcctctcgaagtcaggcttgaagaattcgaactttatgccatagatcgtcttcgagttcttaaaggaatttctgatggtttatctcgaggaaagaaatctgaagaaatggagaaactggttagaggcttgttgaatgcccatatgaaacatccacaggcatccgaggctgtgaacaaggatataatatctcactttgttctgcgcctcgtatactgcagaacggaggacttgagaaaatggtttctttctatggaaactatgctctttcgacaccgttttctttctgaaagtcctgaatctcagaagcaggtcttttcggagcttggcctctcatacaaagcaatcagttatgcagagtttgaggctgtgaaggacaaattggtccaagttgctcggttgattggtcagcctgtaccaagcgctgatgcaatatactataaggtaccatgggaagaagttccagaactggtggctggtcgaagggtattacttcataaaggatatgcatatattgctatttatcaggtggtttcccttgttgcaacacaattccgcagttacctatcaaagaccttaagtctgacaaacaggaaatggacatctacaataagagaacaagagaaggatcggttgacccctatcgttgaagccctttgcacaagctacctgggtcctgactactcacagccaacagagtatgctgatatatcaatcaaagaccttgaacaaattgctaaaagttcatttcctctttgcatgcggcacctatttgataagctgaaagaagatcatcatttgaagcatggagggagaatgcaattaggtctctttctcaagggtgttggtttgaagcttgatgatgcccttactttctggagagctgagttctcgcagagagttggtgttgagaggtttgacaaagaatatgcatacagtatcaggcataactatggaaaagaaggcaagagagtggattattcaccttattcctgtcaaaaaattatctcatcatcacctggtgttggagatcatcatggatgtccctatagacatttcagtgaagacaacttaagagcagctctaggtaaaatgggagtaaataaccagaaaatagaagatatattggacaaagtgcgaaatagacattatcagttggcctgcaccttgacatttgaatcggttcatggttcgtcgtgcgacgctgggattaatcatccaaaccagtacttcattgatagtcaaaaagttctgcaatctaagaacaattcaacagcttag

SEQ ID NO: 15 - protein, 471 aa, Cucumis sativus
  PriL isoform 1 Protein
MARFKLHLFSASVPRSVTAMEPYRSHRKSPISTNSTTTLPLYRSAPPLEVRLEDFELYAIDRLRVLKGISDGLSRGKKSEEMEKLVRELLKTNMKHPQASEVVNKDIISHFVLRLVYCRTEDLRKWFLSMETMLFRHRFLSEGPESQKQVFAELGLSYKAISNAEFEAVRDKLVQVARLIGQPAPSSDAIYYKVPWEEVPELVAGRRVFLHKGYAYIAIYQVVSLVATQFRSYLSKALSLTNRKWTSTIREQEKDRLAPIVEALCTSYLGPDYSQPREYADISIKDLDQIAKSSFPLCMRHLFEKLKEDHHLKHGGRMQLGLFLKGVGLKLDDALAFWRAEFSQRVGAERFDKEYAYSIRHNYGKEGKRVDYSPYSCQKVISSSPSVGDHHGCPYRHFSEDNLRAALGKMGVNNRTMEDIMDKVRNRHYQLACTLTFESIHGSTCDAGINHPNQYFIDSQKVLQSKNNSTS

SEQ ID NO: 16 - DNA, 1416 bp, Cucumis sativus
  PriL isoform 1 CDS
atggcgcgttttaaactccatctattctcagcttcagtgccacgatcagtgactgccatggaaccctatcgttctcacagaaaatctcccatctcaaccaattccactaccactctcccactctaccgctctgctcctcctcttgaagtcaggcttgaagattttgagctttatgccatagatcgtcttcgagttcttaaagggatttctgatgggttatctcgaggaaagaaatctgaagaaatggagaaactggttagagaattgttgaaaaccaacatgaaacatccacaggcatctgaggttgtgaacaaggatataatatctcactttgttctgcgccttgtgtattgccgaacggaggacttgagaaaatggtttctttctatggaaactatgctattccgacatcgttttctttctgaaggtcctgaatctcagaagcaggtatttgcggagcttggtctctcatacaaagcaatcagtaatgcagaatttgaggctgtaagggacaaattggttcaagttgctcggttgattggtcagcctgcaccaagtagtgatgctatatactataaggtaccatgggaagaagttccagagcttgtggctggtcgaagagtattcctccataaaggatatgcatatattgctatttatcaggtggtttcccttgttgcaacacaattccgcagttacctatcgaaggccctaagtctgacgaacaggaaatggacatctacaataagagaacaagagaaagatcggttggccccaatagtagaagccctttgcacgagctacctgggtcctgactactcacagccaagagagtatgcggatatatcaataaaggaccttgaccaaatagctaaaagttcatttccactttgcatgcgacacctatttgaaaagctgaaagaagatcatcatttgaagcatggagggaggatgcaattaggtctctttctcaagggtgttggtttgaagcttgatgatgccctggctttctggagagctgagttctcccagagagttggtgctgagaggtttgacaaagaatatgcatacagtatcaggcataactatggaaaagaaggcaagagagtggattattcgccttattcctgtcaaaaagtaatctcatcatcacctagtgttggagatcatcatggatgtccctatagacatttcagtgaagacaacttaagagcagctcttggtaaaatgggagtaaataaccggacaatggaagatataatggacaaagtgcgaaatagacattatcagttggcatgcaccttgacatttgaatcaatccatggctcgacatgtgatgctgggattaatcatccaaaccagtacttcattgatagtcaaaaggtcctgcaatctaagaataattcaacatcctag

SEQ ID NO: 17 - protein, 468 aa, Cucumis sativus
  PriL isoform 2 Protein
MARFKLHLFSASVPRSVTAMEPYRSHRKSPISTNSTTTLPLYRSAPPLEVRLEDFELYAIDRLRVLKGISDGLSRGKKSEEMEKLVRELLKTNMKHPQASEVVNKDIISHFVLRLVYCRTEDLRKWFLSMETMLFRHRFLSEGPESQVFAELGLSYKAISNAEFEAVRDKLVQVARLIGQPAPSSDAIYYKVPWEEVPELVAGRRVFLHKGYAYIAIYQVVSLVATQFRSYLSKALSLTNRKWTSTIREQEKDRLAPIVEALCTSYLGPDYSQPREYADISIKDLDQIAKSSFPLCMRHLFEKLKEDHHLKHGGRMQLGLFLKGVGLKLDDALAFWRAEFSQRVGAERFDKEYAYSIRHNYGKEGKRVDYSPYSCQKVISSSPSVGDHHGCPYRHFSEDNLRAALGKMGVNNRTMEDIMDKVRNRHYQLACTLTFESIHGSTCDAGINHPNQYFIDSQKVLQSKFGTF

SEQ ID NO: 18 - DNA, 1407 bp, Cucumis sativus
  PriL isoform 2 CDS
atggcgcgttttaaactccatctattctcagcttcagtgccacgatcagtgactgccatggaaccctatcgttctcacagaaaatctcccatctcaaccaattccactaccactctcccactctaccgctctgctcctcctcttgaagtcaggcttgaagattttgagctttatgccatagatcgtcttcgagttcttaaagggatttctgatgggttatctcgaggaaagaaatctgaagaaatggagaaactggttagagaattgttgaaaaccaacatgaaacatccacaggcatctgaggttgtgaacaaggatataatatctcactttgttctgcgccttgtgtattgccgaacggaggacttgagaaaatggtttctttctatggaaactatgctattccgacatcgttttctttctgaaggtcctgaatctcaggtatttgcggagcttggtctctcatacaaagcaatcagtaatgcagaatttgaggctgtaagggacaaattggttcaagttgctcggttgattggtcagcctgcaccaagtagtgatgctatatactataaggtaccatgggaagaagttccagagcttgtggctggtcgaagagtattcctccataaaggatatgcatatattgctatttatcaggtggtttcccttgttgcaacacaattccgcagttacctatcgaaggccctaagtctgacgaacaggaaatggacatctacaataagagaacaagagaaagatcggttggccccaatagtagaagccctttgcacgagctacctgggtcctgactactcacagccaagagagtatgcggatatatcaataaaggaccttgaccaaatagctaaaagttcatttccactttgcatgcgacacctatttgaaaagctgaaagaagatcatcatttgaagcatggagggaggatgcaattaggtctctttctcaagggtgttggtttgaagcttgatgatgccctggctttctggagagctgagttctcccagagagttggtgctgagaggtttgacaaagaatatgcatacagtatcaggcataactatggaaaagaaggcaagagagtggattattcgccttattcctgtcaaaaagtaatctcatcatcacctagtgttggagatcatcatggatgtccctatagacatttcagtgaagacaacttaagagcagctcttggtaaaatgggagtaaataaccggacaatggaagatataatggacaaagtgcgaaatagacattatcagttggcatgcaccttgacatttgaatcaatccatggctcgacatgtgatgctgggattaatcatccaaaccagtacttcattgatagtcaaaaggtcctgcaatctaagtttggaactttctaa

SEQ ID NO: 19 - protein, 439 aa, Citrullus lanatus subsp. vulgaris
  PriL isoform 1 Protein
MEPYRPQGKYSITTSSTTNLPLYRSAPPLEVKLEDFELYAIDRLRVLKGISDGLSRGKKSEEMEKLVRELWNTNMKHPQASEDVNKDIISHFVLRLVYCRTEELRKWFLSMETMLFRHRFLYASSESQKQVFVELGLPYRAISYAEFEAVKDKLVQVARLIGQTVPSGAIYYKVVSLAATQFRSYLSKALSLTNRKWTSTIREQEKDRLTPIVEALCTSYLGPDYSQPREYGDISIKDLDQIAKSSFPLCMRHLFEKLKEDHHLKHGGRMQLGLFLKGVGLKLDDALAFWKAEFSQRVGAERFDKEYAYSIRHNYGKEGKRVDYSPYSCQKIISSSPSVGDHHGCPYRHFSEDNLRAALGKMGVNNRTMDDIMDKVRNRHYQLACTLTFESIHGSSCDAGINHPNQYFIDSQKVLQSKFTYQSLPTADSATHTRLLCLS

SEQ ID NO: 20 - DNA, 1320 bp, Citrullus lanatus subsp. vulgaris
  PriL isoform 1 CDS
atggaaccctatcgtcctcagggaaaatattccatcacaaccagttccactaccaatcttcctctctaccgctctgctcctcctcttgaagtcaagctcgaagatttcgagctttatgctatagatcgtcttcgagttcttaaagggatttctgatggtttatctcgaggaaagaaatctgaagaaatggagaaactggttagagaattgtggaataccaatatgaaacatccacaggcatctgaggatgtgaacaaggatataatctctcactttgttttgcgccttgtatactgcagaacggaggaattgagaaaatggtttctttctatggaaactatgctattccgacaccgttttctttatgcaagttctgaatctcagaagcaggtctttgtggagcttggtctcccatacagagcaatcagttatgcagaatttgaggctgtaaaggacaaattggttcaagttgctcggttgattggtcagactgtaccaagtggtgctatatactataaggtggtttcccttgctgcaacacaattccgcagttacctatcaaaggccctaagtctgacaaacaggaaatggacatctacaataagagaacaagagaaagataggttgaccccaatagtagaagccctttgcacgagctacctgggtcctgactactcacagccaagagagtatggtgatatatcaataaaagaccttgaccaaatagctaaaagttcatttcctctttgcatgcgacacctatttgaaaagctgaaagaagatcatcatttgaagcatggagggaggatgcaattaggtctctttctcaagggtgttggtttgaagcttgatgatgccctggctttctggaaagctgagttctcccagagagttggtgccgagaggtttgacaaagaatatgcatacagtatcaggcataattatggaaaagaaggcaagagagtggattattcgccttattcctgtcaaaaaataatctcatcatcacctagtgttggagatcatcatggatgtccctacagacatttcagtgaagacaacttaagagcagctcttggtaaaatgggagtaaataacaggacaatggacgatataatggacaaagtgcgaaatagacattatcagttggcatgcaccttgacatttgaatcgatccatggctcgtcgtgtgatgctgggattaatcatccaaaccagtacttcattgatagtcagaaggtccttcaatctaagtttacataccaaagcctacctactgcagattcggctacacatactcggttgttgtgtctctcataa

SEQ ID NO: 21 - protein, 452 aa, Citrullus lanatus subsp. vulgaris
  PriL isoform 2 Protein
MEPYRPQGKYSITTSSTTNLPLYRSAPPLEVKLEDFELYAIDRLRVLKGISDGLSRGKKSEEMEKLVRELWNTNMKHPQASEDVNKDIISHFVLRLVYCRTEELRKWFLSMETMLFRHRFLYASSESQKQVFVELGLPYRAISYAEFEAVKDKLVQVARLIGQTVPSGAIYYKVPWEEVPELVAGRRIFLHKGYAYIAMYQVVSLAATQFRSYLSKALSLTNRKWTSTIREQEKDRLTPIVEALCTSYLGPDYSQPREYGDISIKDLDQIAKSSFPLCMRHLFEKLKEDHHLKHGGRMQLGLFLKGVGLKLDDALAFWKAEFSQRVGAERFDKEYAYSIRHNYGKEGKRVDYSPYSCQKIISSSPSVGDHHGCPYRHFSEDNLRAALGKMGVNNRTMDDIMDKVRNRHYQLACTLTFESIHGSSCDAGINHPNQYFIDSQKVLQSKVEHCPH

SEQ ID NO: 22 - DNA, 1359 bp, Citrullus lanatus subsp. vulgaris
  PriL isoform 2 CDS
atggaaccctatcgtcctcagggaaaatattccatcacaaccagttccactaccaatcttcctctctaccgctctgctcctcctcttgaagtcaagctcgaagatttcgagctttatgctatagatcgtcttcgagttcttaaagggatttctgatggtttatctcgaggaaagaaatctgaagaaatggagaaactggttagagaattgtggaataccaatatgaaacatccacaggcatctgaggatgtgaacaaggatataatctctcactttgttttgcgccttgtatactgcagaacggaggaattgagaaaatggtttctttctatggaaactatgctattccgacaccgttttctttatgcaagttctgaatctcagaagcaggtctttgtggagcttggtctcccatacagagcaatcagttatgcagaatttgaggctgtaaaggacaaattggttcaagttgctcggttgattggtcagactgtaccaagtggtgctatatactataaggtaccatgggaagaagttccagagctagtggctggtcgaagaatatttctccataaaggatatgcatatattgctatgtatcaggtggtttcccttgctgcaacacaattccgcagttacctatcaaaggccctaagtctgacaaacaggaaatggacatctacaataagagaacaagagaaagataggttgaccccaatagtagaagccctttgcacgagctacctgggtcctgactactcacagccaagagagtatggtgatatatcaataaaagaccttgaccaaatagctaaaagttcatttcctctttgcatgcgacacctatttgaaaagctgaaagaagatcatcatttgaagcatggagggaggatgcaattaggtctctttctcaagggtgttggtttgaagcttgatgatgccctggctttctggaaagctgagttctcccagagagttggtgccgagaggtttgacaaagaatatgcatacagtatcaggcataattatggaaaagaaggcaagagagtggattattcgccttattcctgtcaaaaaataatctcatcatcacctagtgttggagatcatcatggatgtccctacagacatttcagtgaagacaacttaagagcagctcttggtaaaatgggagtaaataacaggacaatggacgatataatggacaaagtgcgaaatagacattatcagttggcatgcaccttgacatttgaatcgatccatggctcgtcgtgtgatgctgggattaatcatccaaaccagtacttcattgatagtcagaaggtccttcaatctaaggtagaacattgtcctcattga

SEQ ID NO: 23 - protein, 459 aa, Solanum lycopersicum
  PriL Protein
MEAVRSQRKSLVSTGVGSTLPLYRSAPPLEVRLEDFELYAIDRLRVLKGISDALSRGKKPDEMEKLVLDLWKTNMRHQHSSELLNKDIISHFVLRLVYCRTEELRKWFLSIETTLFRYRFRDEPPEKQRALMADFDLPYKAVTIAEYESVKEKLNQVARAIGQPITTDAIYYKVPFEEVPELVAGRRVFIQKGNAYIAMNQVVSLVITQFRSHLSKALVLTNRKWTSMIREQEKDRLVPIIEALSTSYLGPDYNQPREHAEISLKDIDQIAKSSFPLCMGHLFEKLQEDHHLKHGGRMQLGLFLKGVGLKLDDALAFWRAEFSRKVGAERFEKEYAYSIRHNYGKEGKRTDYTPYSCQKIISSTPGVGDHHGCPYRHFSEENLRAALTRMRVGNRALEDVIDKVRNRHYQLACTLTFEAVHGSSCDAGVNHPNQYYNDSQRILESQKSSSNPKGTAASM

SEQ ID NO: 24 - DNA, 1380 bp, Solanum lycopersicum
  PriL CDS
atggaagcggtaagatcgcagcgaaaatctttggtatcgaccggcgtcggttcaacacttcctctctatcgctctgcccctccacttgaagtccgtcttgaagatttcgagctttacgccattgatcgcctccgagttcttaaaggtatttcagatgctttgtctagaggaaagaaacccgatgaaatggagaaattggtgttggatttgtggaaaacaaatatgaggcatcaacattcatctgagctccttaataaggacataatttcacattttgttttgcgacttgtttattgcaggacggaggagttaagaaaatggtttctttcaattgaaactaccttatttcgttaccgtttccgcgatgaacctcctgaaaaacagagagcgctaatggcagattttgatcttccatacaaagctgtaaccattgctgaatacgagagtgtaaaggaaaaattgaaccaagttgcacgcgccataggccaacctattacaactgatgctatctattacaaggttccatttgaggaggtgccagagcttgtggcaggtcgacgagtatttattcaaaaagggaatgcatatatcgccatgaatcaggtggtttcactagtaatcacacagtttcggagtcatctttcgaaagcactagtgctgacaaacagaaaatggacctctatgatcagagaacaggagaaggaccgtttggttcctattattgaagccttatccacaagttacctgggtcctgattataaccagccgagagaacatgcagaaatatcactaaaagacattgaccagattgctaagagttcattccctctatgtatgggtcatcttttcgaaaagctacaagaggatcatcatctgaagcatggagggaggatgcaacttggtctatttctcaagggtgttggattgaagttggatgatgcccttgcattctggagagctgagttctcccgaaaagttggtgctgaaagatttgaaaaagaatatgcatacagcataagacacaactatgggaaagaaggaaagagaacggattacacaccttattcttgtcaaaagattatatcatcaactcctggagttggagatcaccatggctgtccatatcgtcatttcagtgaggagaatctgagagctgctctgaccaggatgagagtaggcaatcgagcactggaggatgtgatagacaaagtccgaaatagacattaccagttggcatgcactttgacctttgaagctgttcatggctcatcttgtgatgccggggttaaccatccaaatcagtactacaatgacagtcagaggatcttggaatcacagaagagttccagtaacccaaaaggaacagcagcttcaatgtag

SEQ ID NO: 25 - protein, 483 aa, Solanum melongena
  PriL Protein
MEAVKSQRKPGVSNGVVSNLPLYLSAPPLEVRLEDFELYAIDRLRVLKGISDALSRGKKPDEMGKLVLDLWETNMRHQHSSEVINKDIISHFVLRLVYCRTEELRKWFLSIETTLFRYRFRDKGPEVQRALMAEFDLPYKAVSNAEYESVKDKLIQVARAIGQHIETNAIFYKVPFEEVPELVAGRRVFIQKGKAYIAMNQVVSLVITKFRSHLSKALVLTNRKWTSMIRVQEKDRLVPIIEALSTSYLGPDYSQPRENAEISLKDIDQIAKSSFPLCMRHLFEKLREDHHLKHGGRMQLGLFLKGVGLKLDDALAFWRAEFSRKVGAERFDKEYAYGIRHIYGKEGKRTVGKKQKKWLLSIFFQFLCSSECPCDYTPYSCQKIISSAPGVGDHHGCPYRHFSEENLRAALTRMRVGNRTLEDVIDKVRNRHYQLACTLTFEAVHSSSCDAGVNHPNQYYNDSQKILESQNSHSKPQGTTASM

SEQ ID NO: 26 - DNA, 1452 bp, Solanum melongena
  PriL CDS
atggaagccgtaaagtctcagaggaaacctggggtatcgaacggcgtcgtttcaaatctccctctctacctctctgcccctcctcttgaagttcgtcttgaagattttgagctttacgccatcgatcgccttcgagttcttaaaggaatttcggatgctttgtctagaggaaagaagccggatgaaatggggaaattggtgttggatctgtgggaaacaaatatgaggcatcaacattcatctgaggttattaataaggacataatttcacatttcgtcttgagacttgtttattgcaggacggaggagttaagaaaatggtttctttctattgaaactaccttatttcgttatcgtttccgggataaaggtcctgaagttcagagggcacttatggcagagtttgatcttccatacaaagctgtgagcaatgctgagtatgaaagtgtgaaggacaaattgatccaagttgcacgcgccataggccaacatattgaaactaatgctatcttttacaaggttccatttgaggaggtgccagagcttgtggcaggtcgaagagtatttattcagaaagggaaggcatatattgccatgaatcaggtggtttcgctagttatcacaaagttccggagtcatctttcgaaagcactagtgttgactaacagaaaatggacctcgatgatcagagtacaagagaaggaccgtttggttcctataattgaagccttatccacaagttacctgggtcctgattatagccagccaagagaaaatgcagaaatatcacttaaagacattgatcagattgctaagagttcattccctctatgtatgcgtcatctttttgaaaagctacgagaggatcatcatctgaagcatggagggaggatgcagcttggtctatttctcaagggtgttggattgaagttggatgatgcccttgcattctggagagctgagttctcccgaaaagttggtgctgaaagatttgacaaagaatatgcatatggcatacgacacatctatgggaaagaaggaaagagaacggtaggaaaaaagcagaagaaatggcttttgtccatattctttcaatttctctgttcctctgaatgcccatgtgattacactccttattcatgtcaaaagattatatcatcagctcctggagtaggagatcaccatggctgtccatatcgtcatttcagtgaggagaacctgagagctgctctgaccaggatgagagtaggcaatcgaacactggaggatgtcatagacaaagtccgaaatagacattaccagttggcatgcactttgacctttgaagctgttcatagctcgtcttgcgatgccggggttaaccatccaaatcagtactacaatgacagtcagaagatcttggaatcacagaatagtcatagcaagccacaaggaacaacagcttcaatgtag

SEQ ID NO: 27 - protein, 488 aa, Capsicum annuum
  PriL isoform 1 Protein
MEAVRSQRKSSFSNGGGGGVSTLPIYRSAPPLEVRLEDFELYAVDRLRVLKRISDGLSRGKKPDEMEKLVLDLWKTNMRHEHSSEVVNKDIISHFVLRLVYCRTEELRKWFLSMETTLFRYRFRDESPEIQRALMAEFDLQYKAVSNAEYESVKDKLNQVARAIGQPITSTDTIFYKNLHRIWSIYIGCFGYSYDQEIWIDWVPFEEVPELVAGRRVLIQKGNAYIAMNQVVSLVITQFRSLLSKALVLTNRKWTSMIREQEKDRLTPIVEALSTSYLGPDYSQPREHAELSLKDIDQIAKSSFPLCMRHLFDKLREDHHLKHGGRMQLGLFLKGVGLKLDDALAFWRAEFSQKVGAERFDKEYAYGIRHNYGKEGKRTDYTPYSCQKIISAAPGVGDHHGCPYRHFSEENLRAALTRMGVGNRAMEDVMDKVRNRHYQLACTLTFEAVHNSTCDAGVNHPNQYFNDSQKIFESKKSSSNPKGAAAST

SEQ ID NO: 28 - DNA, 1467 bp, Capsicum annuum
  PriL isoform 1 CDS
atggaagctgtacgatctcagagaaaatcttcgttttccaatggcggcggcggcggagtttcaactctacctatctatcgctctgctcctcctctcgaagttcgtcttgaagatttcgagctttacgccgtcgatcgcctccgagttctaaaaagaatttcggatggtttgtctagaggaaagaagccagatgaaatggagaaattggtgttggatctatggaaaacaaatatgaggcatgaacattcatctgaggttgttaataaggacataatttcacattttgtcttgcgacttgtttattgcaggacggaggagttaagaaaatggtttctttcaatggaaactaccttatttcgttaccgtttccgggatgagtctcctgaaattcagagggcgctaatggcagagtttgatcttcaatacaaagctgtgagcaatgctgaatatgagagtgtgaaggacaaattgaatcaagttgcacgcgctataggccagcctattacaagcactgatactatcttctacaagaatctgcatcggatttggtctatttatattggatgctttggctacagctatgaccaagaaatatggattgactgggttccattcgaggaggtgccagagcttgtggctggtcgacgagtattaattcagaaagggaatgcatatattgccatgaatcaggtggtttcactagttatcacacagttccgaagtcttctttccaaagcactggtgctgacaaacagaaaatggacttcgatgatcagagaacaggagaaggaccgtttgactcctattgttgaagccttatccacaagttacctgggtcctgattatagccagccaagagaacatgcagaactatcactaaaagacattgatcagattgctaagagttcattccctctgtgtatgcgtcatcttttcgataagctacgtgaggatcatcatctgaaacacggagggaggatgcaacttggactatttctcaagggtgttggattgaagttggatgatgcccttgcattctggagagctgagttctcccagaaagttggtgctgaaagatttgataaagaatatgcatacggcataagacacaactatgggaaagaaggaaagagaacggactacacaccttattcttgtcaaaagattatatcggctgctcctggagttggagatcaccatggctgtccatatcgtcatttcagtgaggagaatctgagagctgctctgaccaggatgggagtaggcaatcgagcaatggaggatgtgatggacaaagtccgaaatagacattatcagttggcatgcactttgacctttgaagctgttcacaactcaacttgtgatgcgggggttaaccatccaaatcagtacttcaatgatagtcagaagatctttgaatcaaagaaaagttccagcaacccaaaaggagcagcagcgtcaacgtag

SEQ ID NO: 29 - protein, 463 aa, Capsicum annuum
  PriL isoform 2 Protein
MEAVRSQRKSSFSNGGGGGVSTLPIYRSAPPLEVRLEDFELYAVDRLRVLKRISDGLSRGKKPDEMEKLVLDLWKTNMRHEHSSEVVNKDIISHFVLRLVYCRTEELRKWFLSMETTLFRYRFRDESPEIQRALMAEFDLQYKAVSNAEYESVKDKLNQVARAIGQPITSTDTIFYKVPFEEVPELVAGRRVLIQKGNAYIAMNQVVSLVITQFRSLLSKALVLTNRKWTSMIREQEKDRLTPIVEALSTSYLGPDYSQPREHAELSLKDIDQIAKSSFPLCMRHLFDKLREDHHLKHGGRMQLGLFLKGVGLKLDDALAFWRAEFSQKVGAERFDKEYAYGIRHNYGKEGKRTDYTPYSCQKIISAAPGVGDHHGCPYRHFSEENLRAALTRMGVGNRAMEDVMDKVRNRHYQLACTLTFEAVHNSTCDAGVNHPNQYFNDSQKIFESKKSSSNPKGAAAST

SEQ ID NO: 30 - DNA, 1392 bp, Capsicum annuum
  PriL isoform 2 CDS
atggaagctgtacgatctcagagaaaatcttcgttttccaatggcggcggcggcggagtttcaactctacctatctatcgctctgctcctcctctcgaagttcgtcttgaagatttcgagctttacgccgtcgatcgcctccgagttctaaaaagaatttcggatggtttgtctagaggaaagaagccagatgaaatggagaaattggtgttggatctatggaaaacaaatatgaggcatgaacattcatctgaggttgttaataaggacataatttcacattttgtcttgcgacttgtttattgcaggacggaggagttaagaaaatggtttctttcaatggaaactaccttatttcgttaccgtttccgggatgagtctcctgaaattcagagggcgctaatggcagagtttgatcttcaatacaaagctgtgagcaatgctgaatatgagagtgtgaaggacaaattgaatcaagttgcacgcgctataggccagcctattacaagcactgatactatcttctacaaggttccattcgaggaggtgccagagcttgtggctggtcgacgagtattaattcagaaagggaatgcatatattgccatgaatcaggtggtttcactagttatcacacagttccgaagtcttctttccaaagcactggtgctgacaaacagaaaatggacttcgatgatcagagaacaggagaaggaccgtttgactcctattgttgaagccttatccacaagttacctgggtcctgattatagccagccaagagaacatgcagaactatcactaaaagacattgatcagattgctaagagttcattccctctgtgtatgcgtcatcttttcgataagctacgtgaggatcatcatctgaaacacggagggaggatgcaacttggactatttctcaagggtgttggattgaagttggatgatgcccttgcattctggagagctgagttctcccagaaagttggtgctgaaagatttgataaagaatatgcatacggcataagacacaactatgggaaagaaggaaagagaacggactacacaccttattcttgtcaaaagattatatcggctgctcctggagttggagatcaccatggctgtccatatcgtcatttcagtgaggagaatctgagagctgctctgaccaggatgggagtaggcaatcgagcaatggaggatgtgatggacaaagtccgaaatagacattatcagttggcatgcactttgacctttgaagctgttcacaactcaacttgtgatgcgggggttaaccatccaaatcagtacttcaatgatagtcagaagatctttgaatcaaagaaaagttccagcaacccaaaaggagcagcagcgtcaacgtag

SEQ ID NO: 31 - protein, 410 aa, Capsicum annuum
  PriL isoform 3 Protein
MEAVRSQRKSSFSNGGGGGVSTLPIYRSAPPLEVRLEDFELYAVDRLRVLKRISDGLSRGKKPDEMEKLVLDLWKTNMRHEHSSEVVNKDIISHFVLRLVYCRTEELRKWFLSMETTLFRYRFRDESPEIQRALMAEFDLQYKAVSNAEYESVKDKLNQVARAIGQPITSTDTIFYKNLHRIWSIYIGCFGYSYDQEIWIDWVPFEEVPELVAGRRVLIQKGNAYIAMNQVVSLVITQFRSLLSKALVLTNRKWTSMIREQEKDRLTPIVEALSTSYLGPDYSQPREHAELSLKDIDQIAKSSFPLCMRHLFDKLREDHHLKHGGRMQLGLFLKGVGLKLDDALAFWRAEFSQKVGAERFDKEYAYGIRHNYGKEGKRTSAGLHTLFLSKDYIGCSWSWRSPWLSISSFQ

SEQ ID NO: 32 - DNA, 1233 bp, Capsicum annuum
  PriL isoform 3 CDS
atggaagctgtacgatctcagagaaaatcttcgttttccaatggcggcggcggcggagtttcaactctacctatctatcgctctgctcctcctctcgaagttcgtcttgaagatttcgagctttacgccgtcgatcgcctccgagttctaaaaagaatttcggatggtttgtctagaggaaagaagccagatgaaatggagaaattggtgttggatctatggaaaacaaatatgaggcatgaacattcatctgaggttgttaataaggacataatttcacattttgtcttgcgacttgtttattgcaggacggaggagttaagaaaatggtttctttcaatggaaactaccttatttcgttaccgtttccgggatgagtctcctgaaattcagagggcgctaatggcagagtttgatcttcaatacaaagctgtgagcaatgctgaatatgagagtgtgaaggacaaattgaatcaagttgcacgcgctataggccagcctattacaagcactgatactatcttctacaagaatctgcatcggatttggtctatttatattggatgctttggctacagctatgaccaagaaatatggattgactgggttccattcgaggaggtgccagagcttgtggctggtcgacgagtattaattcagaaagggaatgcatatattgccatgaatcaggtggtttcactagttatcacacagttccgaagtcttctttccaaagcactggtgctgacaaacagaaaatggacttcgatgatcagagaacaggagaaggaccgtttgactcctattgttgaagccttatccacaagttacctgggtcctgattatagccagccaagagaacatgcagaactatcactaaaagacattgatcagattgctaagagttcattccctctgtgtatgcgtcatcttttcgataagctacgtgaggatcatcatctgaaacacggagggaggatgcaacttggactatttctcaagggtgttggattgaagttggatgatgcccttgcattctggagagctgagttctcccagaaagttggtgctgaaagatttgataaagaatatgcatacggcataagacacaactatgggaaagaaggaaagagaacgagtgcaggactacacaccttattcttgtcaaaagattatatcggctgctcctggagttggagatcaccatggctgtccatatcgtcatttcagtga

(19) United States
(12) Patent Application Publication
(10) Pub. No.: US 2022/0267791 A1
(43) Pub. Date: Aug. 25, 2022

(54) BEGOMOVIRUS RESISTANCE RELATED GENES

(71) Applicant: RIJK ZWAAN ZAADTEELT EN ZAADHANDEL B.V., De Lier (NL)

(72) Inventors: Valentina BRACUTO, De Lier (NL); Adrianus Cornelis KOEKEN, De Lier (NL); Florian MULLER, De Lier (NL); Emilio SARRIA VILLADA, De Lier (NL)

(21) Appl. No.: 17/699,346

(22) Filed: Mar. 21, 2022

Related U.S. Application Data
(63) Continuation-in-part of application No. PCT/EP2020/077558, filed on Oct. 1, 2020.
Application No. PCT/EP2020/077558 is a continuation-in-part of application No. PCT/EP2019/076611, filed on Oct. 1, 2019.

(51) Int. Cl. C12N 15/82; C07K 14/415

(52) U.S. Cl. CPC C12N 15/8283; C07K 14/415

(57) ABSTRACT

The present invention relates to a modified PriL gene, which encodes a modified protein which may comprise one or more modifications in the wild type protein sequence of SEQ ID NO: 1 or SEQ ID NO: 3 or in a protein sequence having at least 60% sequence identity to SEQ ID NO: 1 or SEQ ID NO: 3. Alternatively, the wild type of the modified PriL gene encodes a protein according to SEQ ID NO: 1, SEQ ID NO: 3, SEQ ID NO: 7, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, or SEQ ID NO: 31. The modification in the wild type protein sequence is an amino acid substitution.

RELATED APPLICATIONS AND INCORPORATION BY REFERENCE

This application is a continuation-in-part application of international patent application Ser. No. PCT /EP2020/077558 filed 1 Oct. 2020, which published as PCT Publication No. WO 2021/064118 on 8 Apr. 2021, which claims benefit of international patent application Serial No. PCT/EP2019/076611 filed 1 Oct. 2019.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said ASCII copy was created March 17, 2020, is named Y7954-00519SL.txt and is 91.724 bytes in size.

### FIELD OF THE INVENTION

The present invention relates to a modified gene that imparts Begomovirus resistance in a plant of the Cucurbitaceae or Solanaceae family.

### BACKGROUND OF THE INVENTION

Begomoviruses form a genus of viruses in the family Geminiviridae that exhibit a wide host range in a number of economically valuable crop species including those of the Cucurbitaceae (e.g. Cucurbita moschata, Cucurbita pepo, Cucumis melo, Cucumis sativus, Cucurbita maxima, Citrullus lanatus) and Solanaceae (e.g. Solanum lycopersicum, Solanum melongena, and Capsicum annuum) families. Currently there are over 300 species classified as Begomoviruses, including Tomato Leaf Curl New Delhi Virus (ToLCNDV), Tomato Leaf Curl Palampur Virus (ToLCPMV), Cucurbit Leaf Curl Virus (CuLCV), Melon Chlorotic Leaf Curl Virus (MCLCV), Melon Leaf Curl Virus (MLCV), Squash Leaf Curl Virus (SqLCV), Cucumber Leaf Crumple Virus (CuLCrV), Tomato Yellow Leaf Curl Virus (TYLCV), Watermelon Chlorotic Stunt Virus (WmCSV), and Watermelon Curly Mottle Virus (WmCMoV). Begomoviruses are transmitted by an insect vector, which can be the white fly Bemisia tabaci or other whiteflies. Disease symptoms typically manifest in infected plants as leaf chlorosis, mottled or mosaic leaves, leaf curling or distortion, and stunting of the plant. Fruits grown from Begomovirus infected plants may have symptoms ranging from rough skin, longitudinal cracking, dehydration and speckling. Plants infected with the virus at an early stage may be severely stunted and fruit production may be affected, if not suppressed.

With exceptionally high yield losses attributed to Begomovirus infection, preventing infections from occurring and spreading have become of utmost importance. Currently, control measures against some Begomoviruses are limited and mainly rely on various cultural, phytosanitary and hygienic practices to control whiteflies, including biological control or chemical treatments of whiteflies, cultivation of plants under insect-proof greenhouses, and the elimination of infected plants.

Plant viruses multiply inside their host cells. The genome of Begomoviruses such as ToLCNDV, ToLCPMV, CuLCV, MCLCV, MLCV, SqLCV, CuLCrV, TYLCV, WmCSV and WmCMoV consists of one (monopartite) or two (bipartite) DNA molecules that are individually encapsidated in a virion. Virus and host plant interaction studies have shown that important viral proteins interact with host proteins, leading to the increase of viral DNA. Thus Begomoviruses heavily rely on the host cell replication machinery for replication and spreading.

The genetic basis of Begomovirus resistance is not well characterized.

Citation or identification of any document in this application is not an admission that such document is available as prior art to the present invention.

### SUMMARY OF THE INVENTION

It is thus an object of the present invention to provide a gene that leads to Begomovirus resistance, in particular, resistance to one or more of the following Begomoviruses selected from ToLCNDV and/or ToLCPMV and/or CuLCV and/or MCLCV and/or MLCV and/or SqLCV and/or CuLCrV and/or TYLCV and/or WmCSV and/or WmCMoV, in a plant of the Cucurbitaceae or Solanaceae family, in particular Cucurbita moschata (C. moschata), Cucurbita pepo (C. pepo), Cucumis melo (C. melo), Cucumis sativus (C. sativus), Cucurbita maxima (C. maxima), Citrullus lanatus (C. lanatus), Solanum lycopersicum (S. lycopersicum), Solanum melongena (S. melongena) and Capsicum annuum (C. annuum) plants.

In the research that led to the present invention, novel plants of the Cucurbitaceae family, in particular C. pepo and C. melo plants, were developed that are highly resistant to Begomoviruses, and in particular, highly resistant to ToLCNDV. It was surprisingly found that resistance resulted from modifications in the DNA Primase Large Subunit gene, abbreviated herein as PriL.

The present invention relates to a modified gene that imparts Begomovirus resistance in a plant of the Cucurbitaceae or Solanaceae family. The invention further relates to a plant which may comprise the modified gene and to progeny, seed, and fruit of the Begomovirus resistant plant. The invention also relates to propagation material suitable for producing the Begomovirus resistant plant. Additionally, the invention relates to use of the modified gene for producing Begomovirus resistant plants, as well as methods for identifying and selecting a plant of the Cucurbitaceae or Solanaceae family having resistance against Begomoviruses.

The present invention relates to a modified PriL gene, which encodes a modified protein which may comprise one or more modifications in the wild type protein sequence of SEQ ID NO: 1 or SEQ ID NO: 3 or in a protein sequence having at least 60% sequence identity to SEQ ID NO: 1 or SEQ ID NO: 3. Alternatively, the wild type of the modified PriL gene encodes a protein according to SEQ ID NO: 1, SEQ ID NO: 3, SEQ ID NO: 7, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, or SEQ ID NO: 31. The modification in the wild type protein sequence is an amino acid substitution, in particular a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5, and/or wherein the modified gene encodes a modified protein that may comprise a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DEPOSITS

Seeds of a Cucumis melo plant comprising the modified PriL gene of the invention homozygously in its genome and which confers resistance to ToLCNDV were deposited under accession number NCIMB 43372 on 19 Mar., 2019 with NCIMB Ltd. (Ferguson Building, Craibstone Estate, Bucksburn, Aberdeen, AB21 9YA).

Seeds of a Cucurbita pepo plant comprising the modified PriL gene of the invention homozygously in its genome and which confers resistance to ToLCNDV were also deposited under accession number NCIMB 43405 on 23 May, 2019 with NCIMB Ltd.

The Deposits with NCIMB Ltd., under deposit accession numbers 43372 and 43405 were made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1: Multiple sequence alignment of orthologous PriL protein sequences from Table 1 of Cucurbita moschata primase isoform 1—SEQ ID NO: 1; Cucurbita moschata primase isoform 2—SEQ ID NO: 3; Cucumis melo primase—SEQ ID NO: 7; Cucurbita pepo primase—SEQ ID NO: 11; Cucurbita maxima primase—SEQ ID NO: 13; Cucumis sativus primase isoform 1—SEQ ID NO: 15; Cucumis sativus primase isoform 2—SEQ ID NO: 17; Citrullus lanatus primase isoform 1—SEQ ID NO: 19; Citrullus lanatus primase isoform 2—SEQ ID NO: 21; Solanum lycopersicum primase—SEQ ID NO: 23, Solanum melongena primase—SEQ ID NO: 25; Capsicum annuum primase isoform 1—SEQ ID NO: 27; Capsicum annuum primase isoform 2-SEQ ID NO: 29; and Capsicum annuum primase isoform 3—SEQ ID NO: 31. The following symbols are used below the alignment: * all residues in that column are identical, : conserved substitutions have been observed, . semi-conserved substitutions have been observed, and no match (space).

FIG. 2: Sequence identity and sequence similarity of the PriL proteins from Table 1 of Cucurbita moschata primase isoform 1—SEQ ID NO: 1; Cucurbita moschata primase isoform 2—SEQ ID NO: 3; Cucumis melo primase—SEQ ID NO: 7; Cucurbita pepo primase—SEQ ID NO: 11; Cucurbita maxima primase—SEQ ID NO: 13; Cucumis sativus primase isoform 1—SEQ ID NO: 15; Cucumis sativus primase isoform 2—SEQ ID NO: 17; Citrullus lanatus primase isoform 1—SEQ ID NO: 19; Citrullus lanatus primase isoform 2—SEQ ID NO: 21; Solanum lycopersicum primase—SEQ ID NO: 23, Solanum melongena primase—SEQ ID NO: 25; Capsicum annuum primase isoform 1—SEQ ID NO: 27; Capsicum annuum primase isoform 2—SEQ ID NO: 29; and Capsicum annuum primase isoform 3—SEQ ID NO: 31. Sequence identity is calculated using a method taking the gaps into account; sequence similarity is calculated based on grouping of amino acids having similar properties. See SIAS method for both options.

FIGS. 3A and 3B: Examples of an InterProScan output for the functional analysis and classification of orthologous PriL protein sequences of C. moschata PriL isoform 1—SEQ ID NO: 1 and C. melo PriL—SEQ ID NO: 7. All PriL orthologs identified as part of this invention belong to the DNA primase large subunit, eukaryotic (IPRO16558) family. PriL is predicted to have a role in the biological process of synthesizing short RNA primers from which DNA polymerases extend during DNA replication (Gene Ontology Accession: GO:0006269). It has DNA primase activity (Gene Ontology Accession: GO0003896).

FIG. 4A: Predicted wild type C. moschata PriL protein structure; FIG. 4B: Predicted modified C. moschata PriL protein structure; In both predictions, the amino acids at position 147, 162 and 163 are visualized. The amino acid substitutions F147C, G162D, and Q163H are predicted to be located at the exposed surface of the modified PriL protein.

FIG. 5A: Predicted wild type C. melo PriL protein structure; FIG. 5B: Predicted modified C. melo PriL protein structure; In both predictions, the amino acids at position 166 and 200 are visualized (amino acids at position 4 not pictured). The amino acid substitutions Y4H, P166Q, and I200T, are predicted to be located at the exposed surface of the modified PriL protein.

FIG. 6: The mean ToLCNDV disease score for C. moschata 109238, C. pepo spp. pepo cv. 10006 and C. pepo spp. pepo cv. Zucchini MU-CU-16 for each of the years between 2016-2019.

### DETAILED DESCRIPTION OF THE INVENTION

In general, DNA primase is an RNA polymerase that is an essential component needed for the replication of DNA. DNA primase synthesizes short RNA primers that replicative polymerases use to initiate DNA synthesis and elongate therefrom. All bacteria, eukaryotes and many viruses are known to initiate DNA synthesis using the short RNA primers synthesized by DNA primase. Primases are grouped into two classes, bacteria/bacteriophage and archaeal/eukaryotic. Eukaryotic DNA primase is a heterodimeric protein complex which may comprise a large subunit, PriL, and a small subunit (abbreviated herein as PriS). PriS is expected to have a catalytic function, while several functions have been proposed for PriL, including stabilization of PriS, involvement in synthesis initiation, improvement of Primase processivity, determination of product size and the transfer of the products to DNA polymerase alpha. Studies in yeast for example, have provided evidence that the PriL subunit is essential for DNA Primase function, as the disruption of the PriL gene in yeast is lethal.

Begomoviruses like ToLCNDV, ToLCPMV, CuLCV, MCLCV, MLCV, SqLCV, CuLCrV, TYLCV, WmCSV and WmCMoV multiply by a mechanism known as rolling circle replication (RCR) and this mechanism is heavily reliant on host replication proteins. Without being bound by theory, it is hypothesized by the present inventors that modifications to the PriL gene lead to an alteration in the DNA primase protein such that virus replication proteins are altered in their ability to interact with host DNA Primase for the purpose of viral replication. This in turn provides the plant with resistance to the virus. Modifications to the PriL gene and/or encoded protein that were identified in the present invention, are not expected by the inventors to lead to the complete loss of function of the PriL protein, otherwise it is expected to be lethal to the plant since this is a single copy gene and one that encodes an essential protein for plant cellular DNA replication. Modifications to the PriL gene, and the resultant resistance to Begomoviruses, like those that were found in the present research, would be widely applicable to members of the Cucurbitaceae and Solanaceae families in which an orthologous PriL gene with a similar function exists.

The present invention is thus broadly applicable to members of the Cucurbitaceae and Solanaceae families that comprise a PriL gene and are naturally susceptible to Begomoviruses, in particular, to one or more of the following Begomoviruses selected from the group consisting of ToLCNDV, ToLCPMV, CuLCV, MCLCV, MLCV, SqLCV, CuLCrV, TYLCV, WmCSV and WmCMoV.

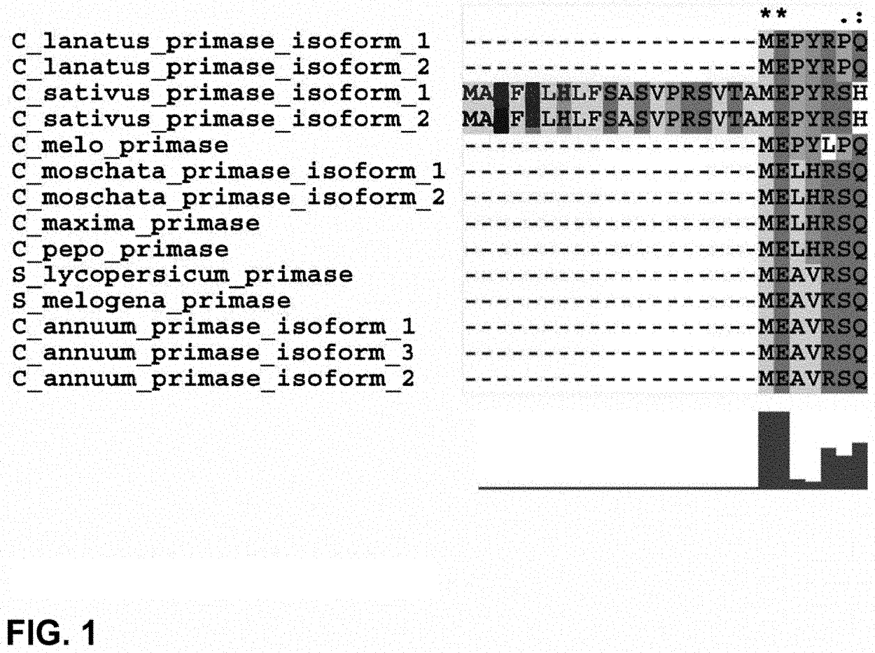

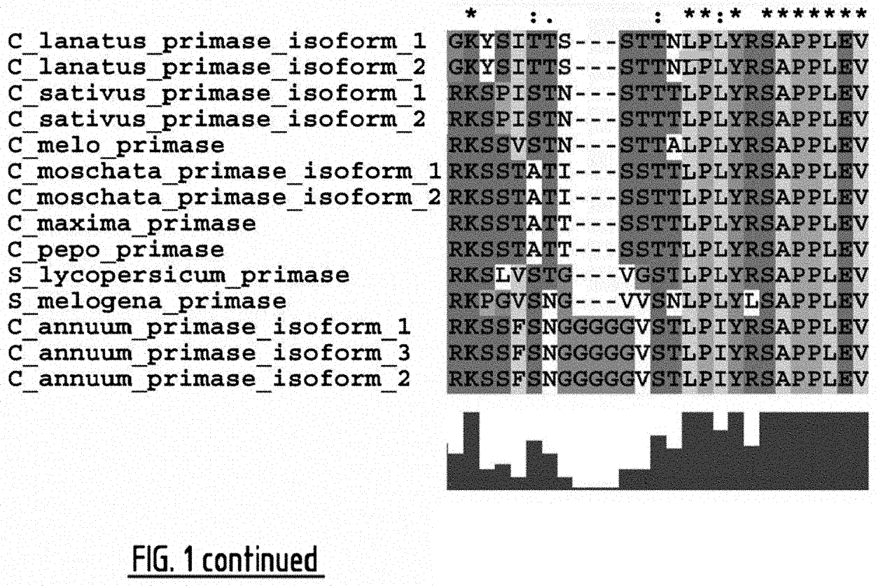

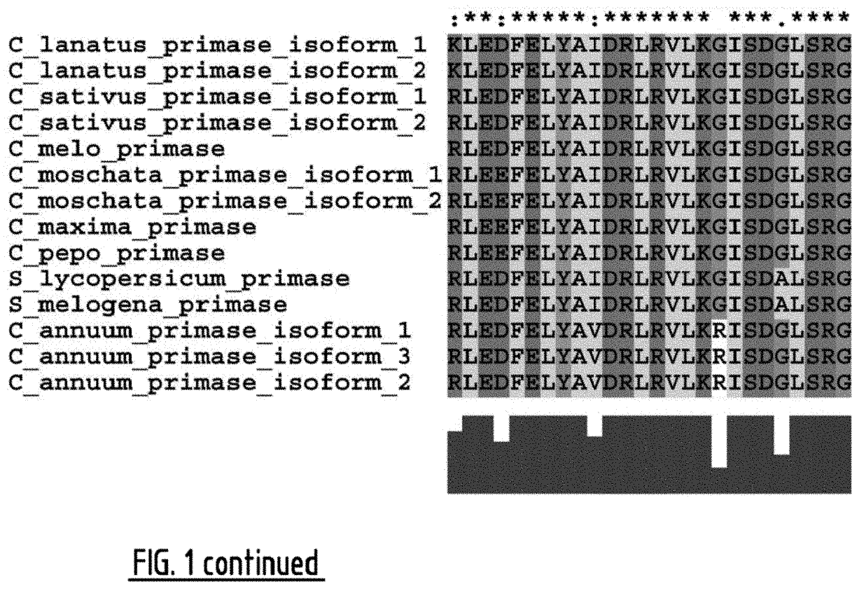

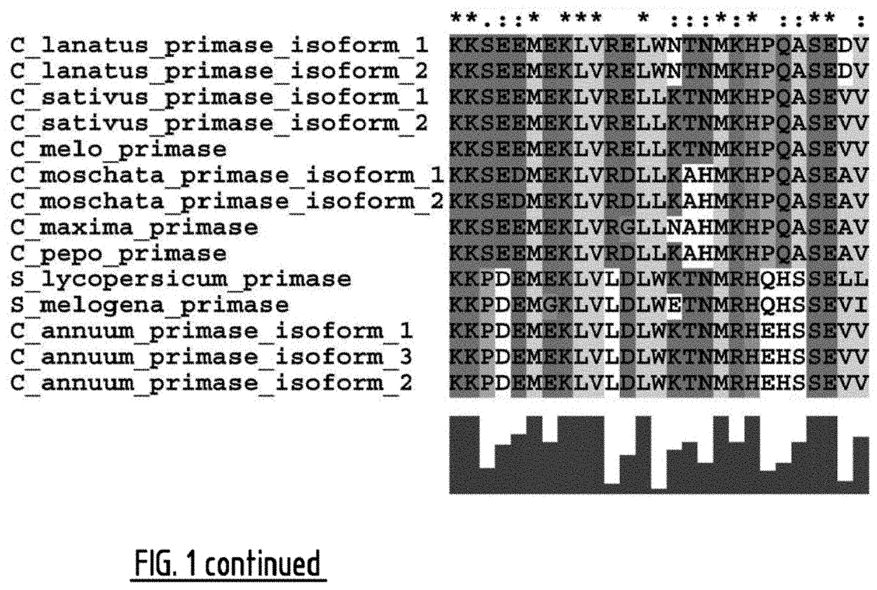

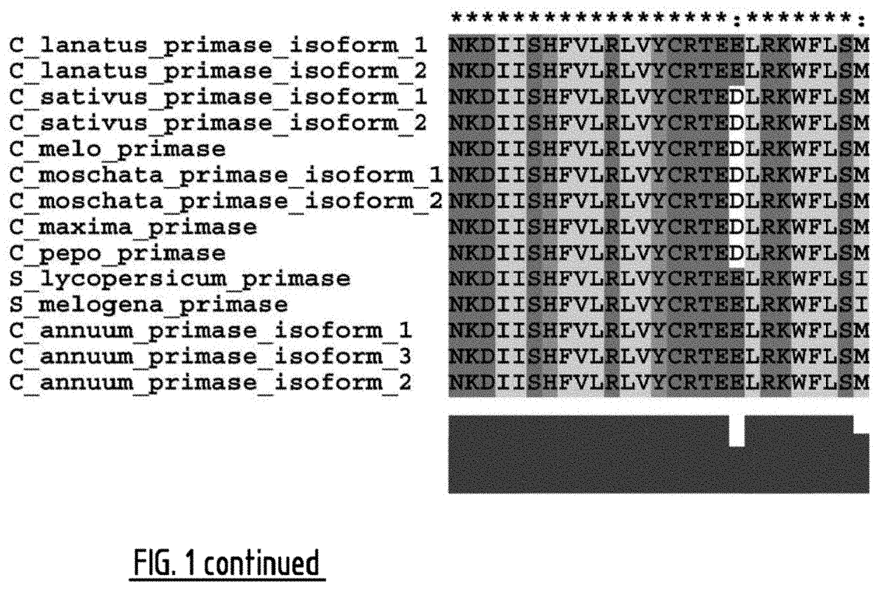

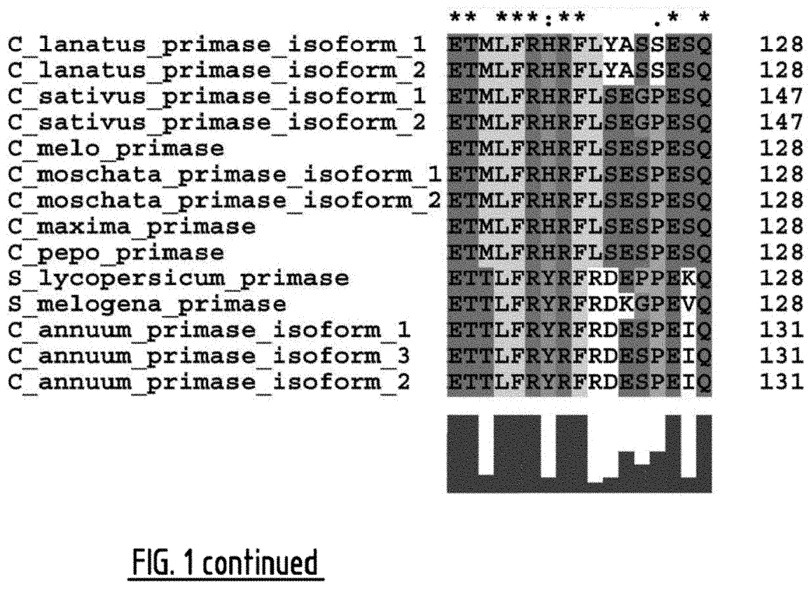

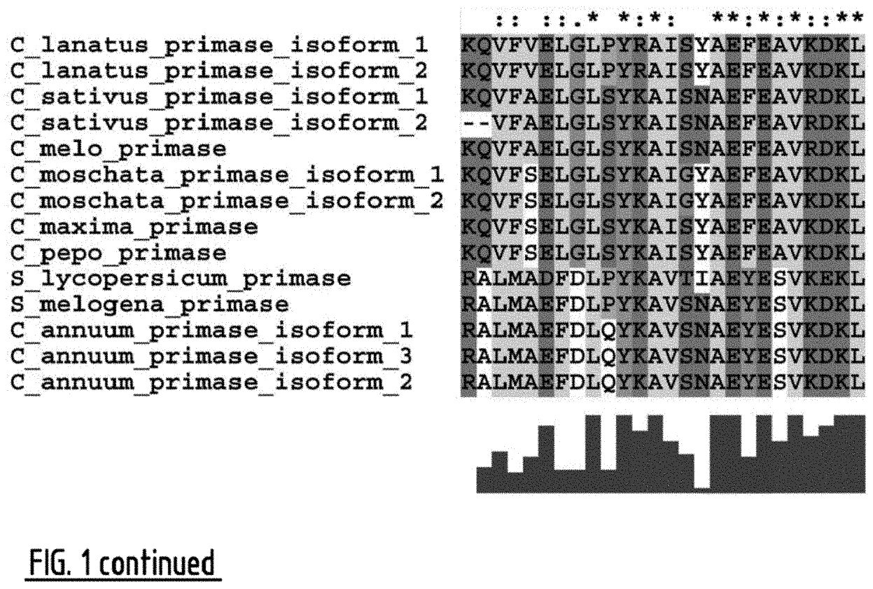

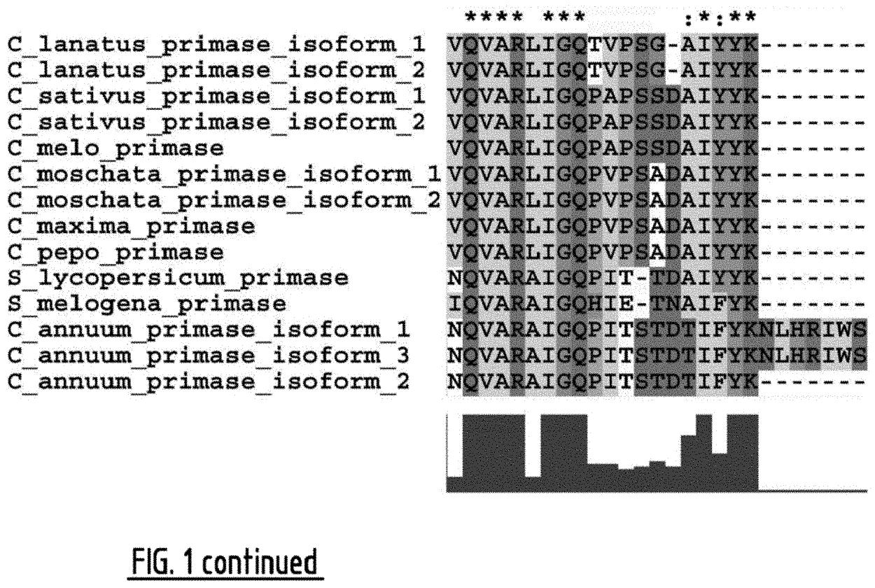

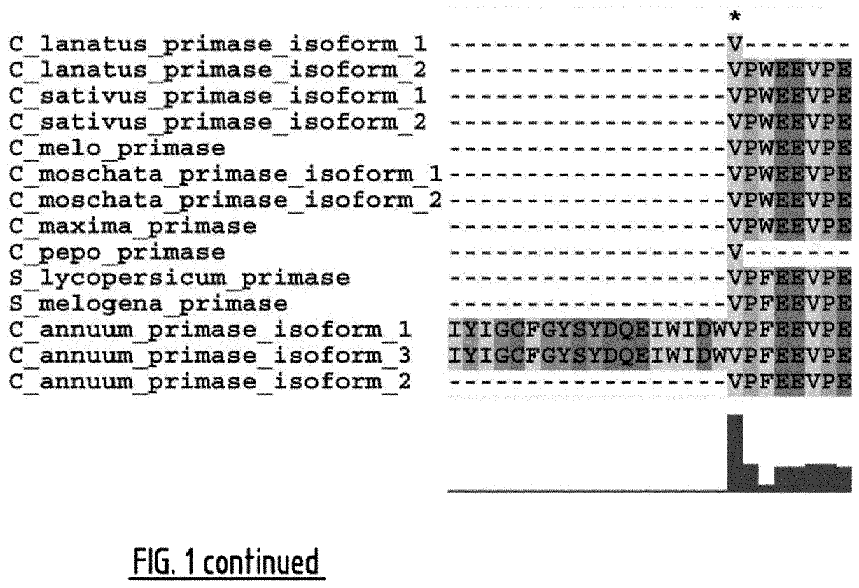

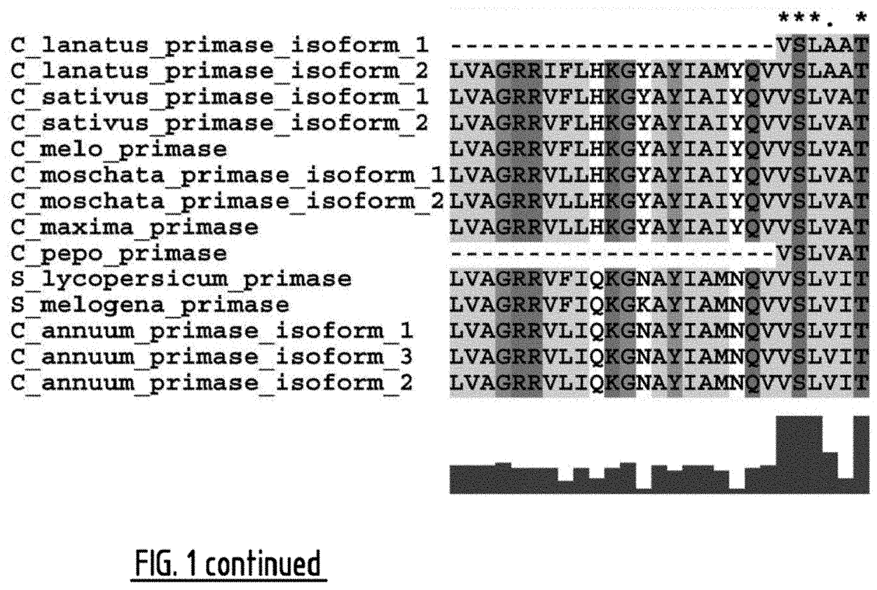

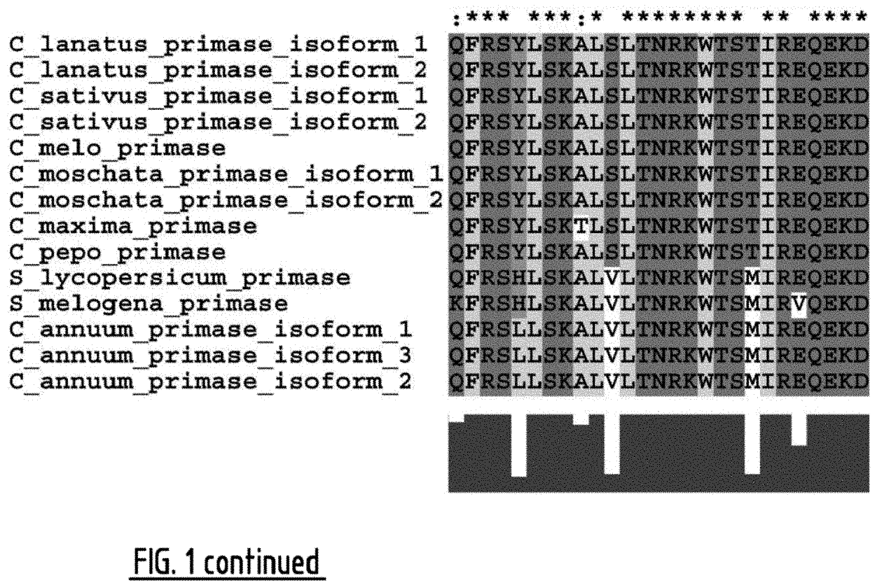

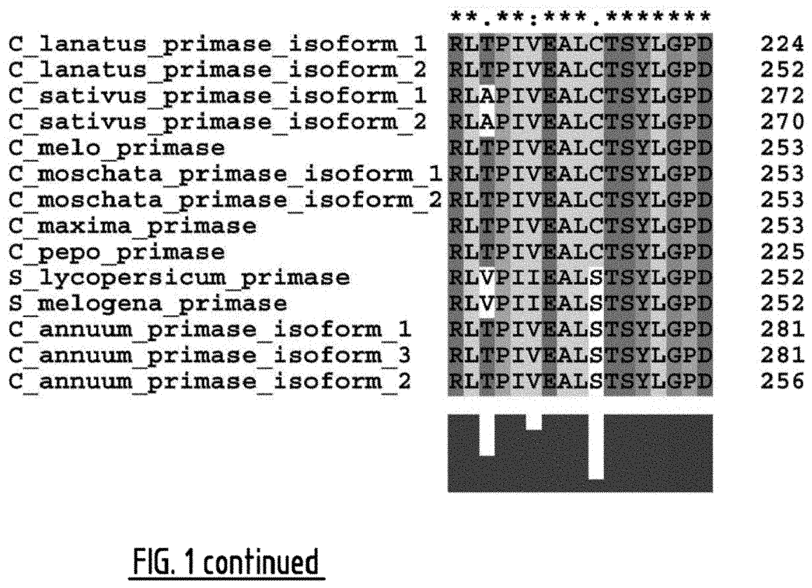

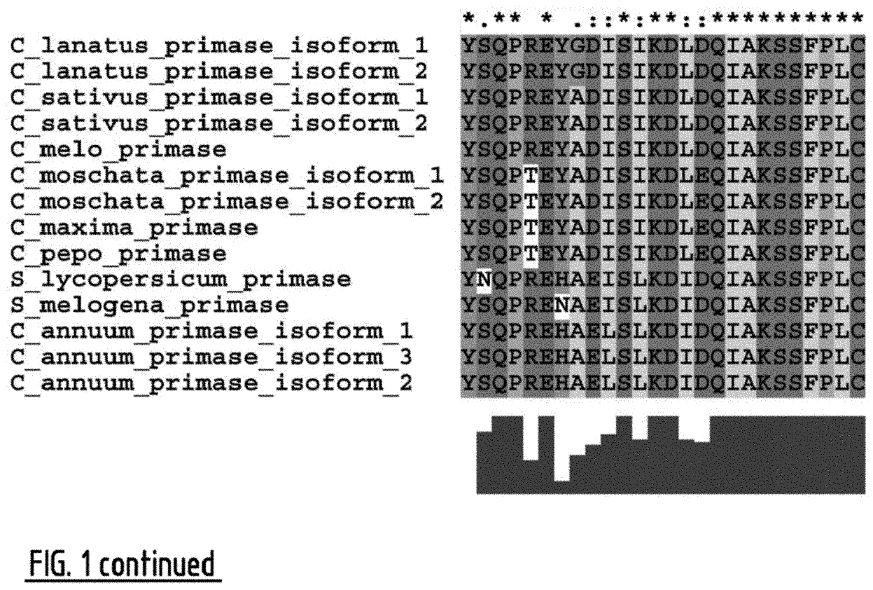

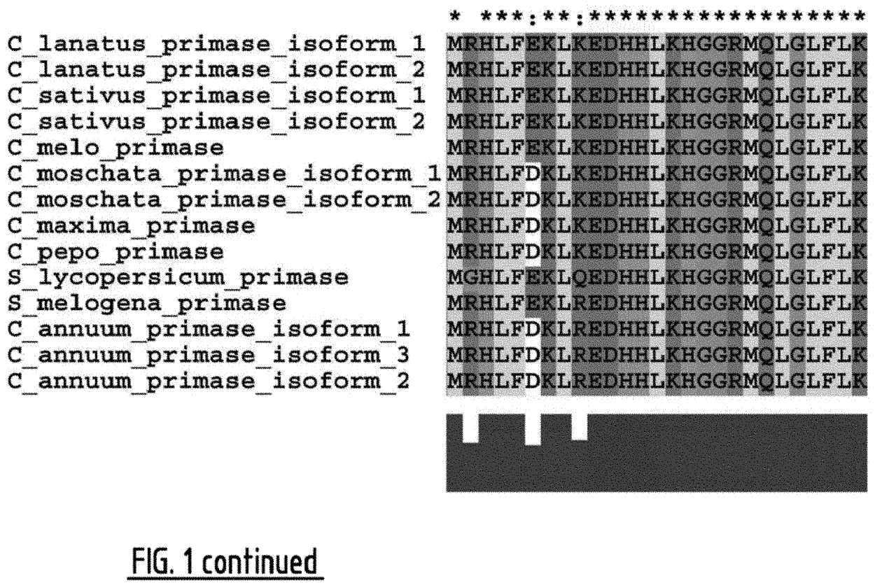

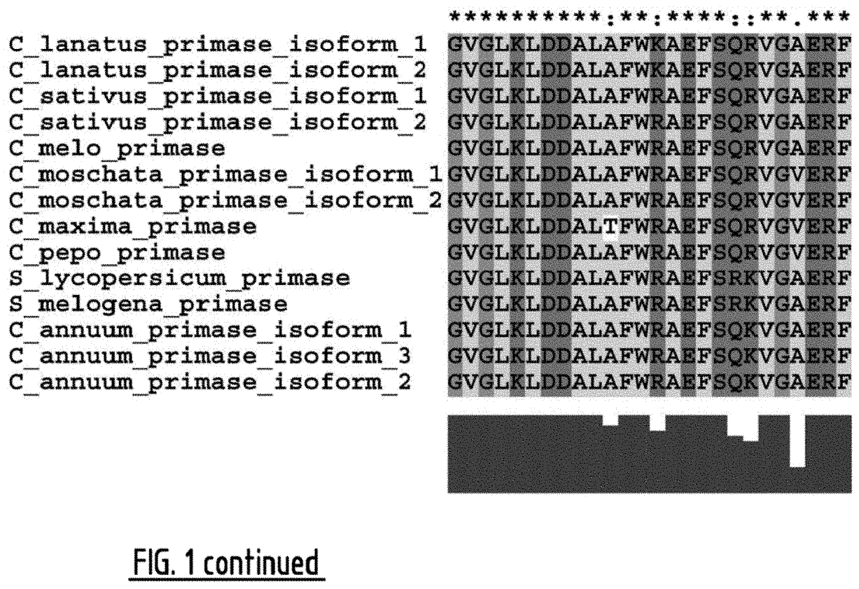

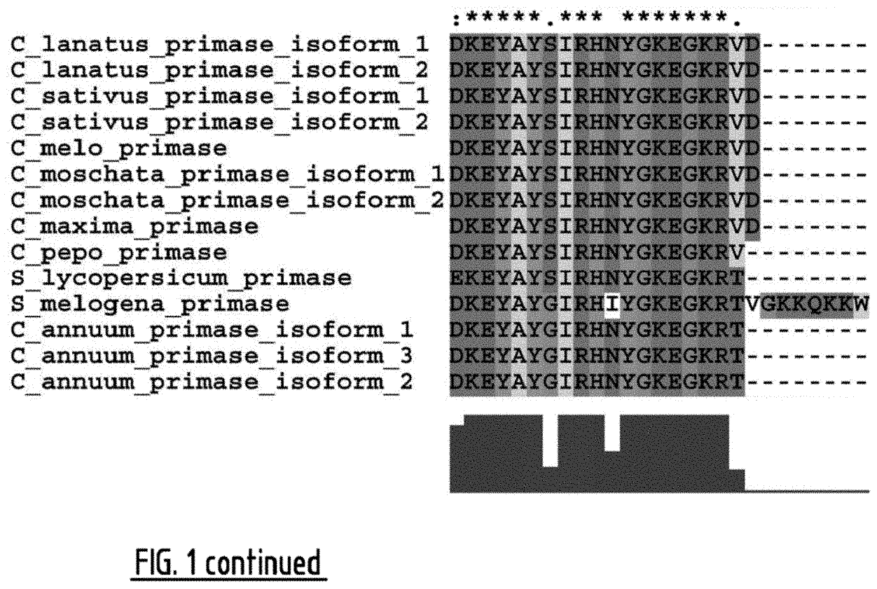

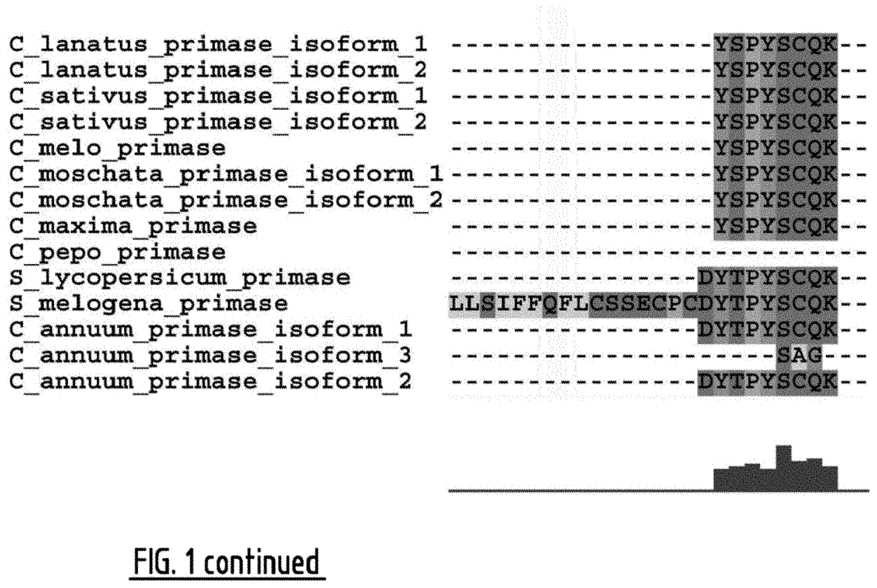

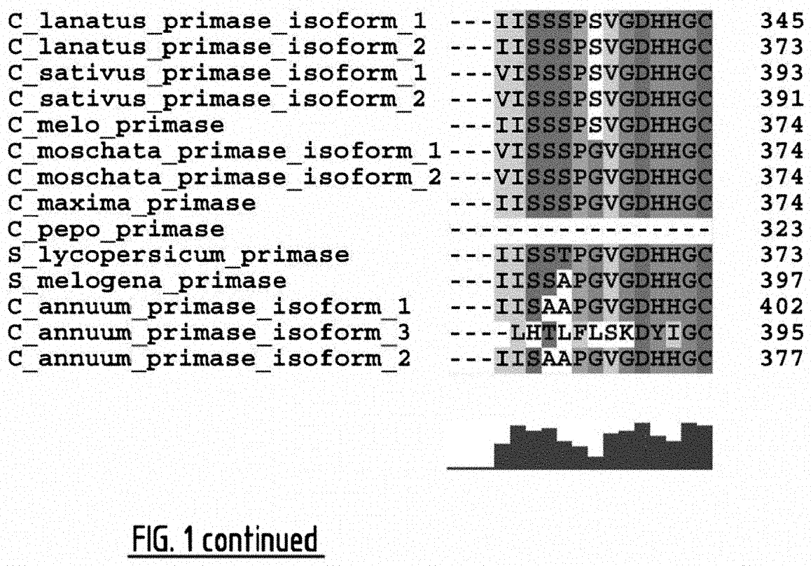

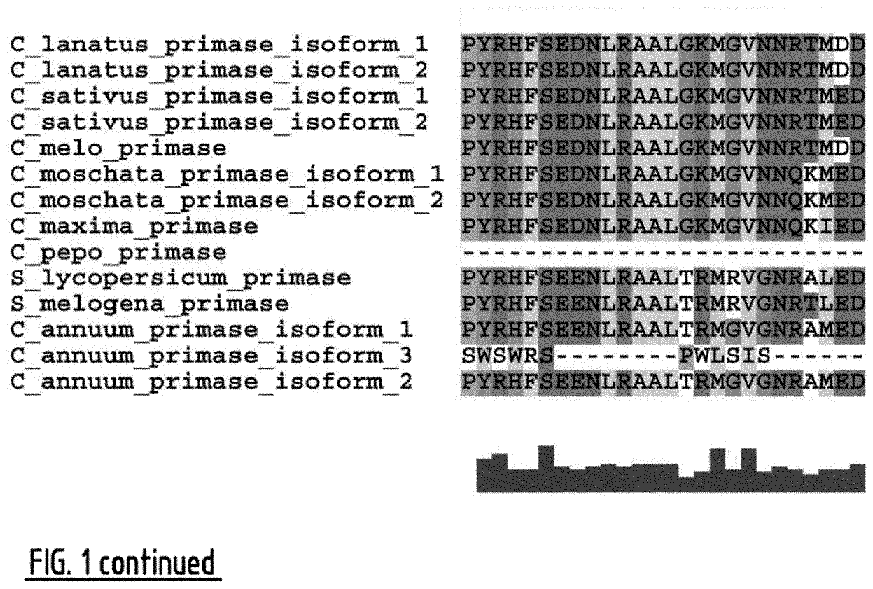

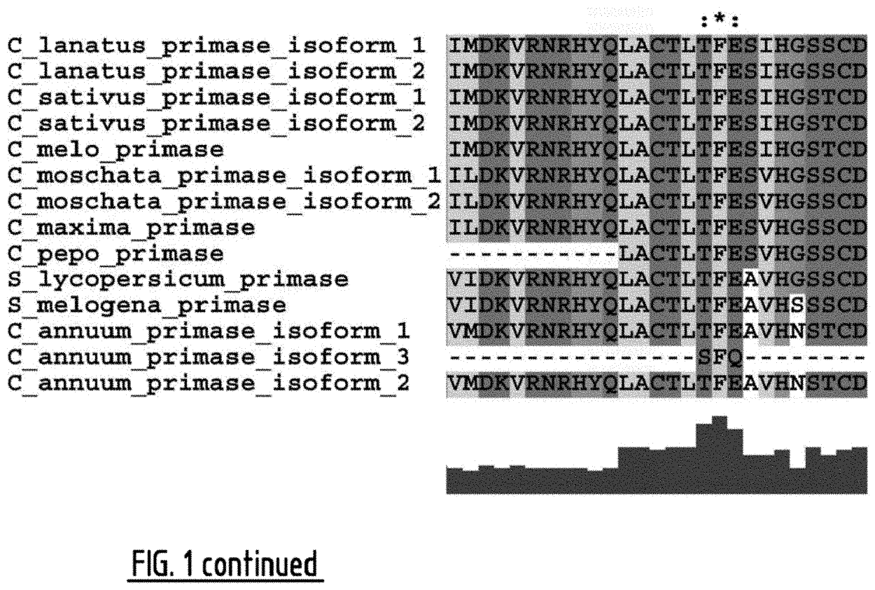

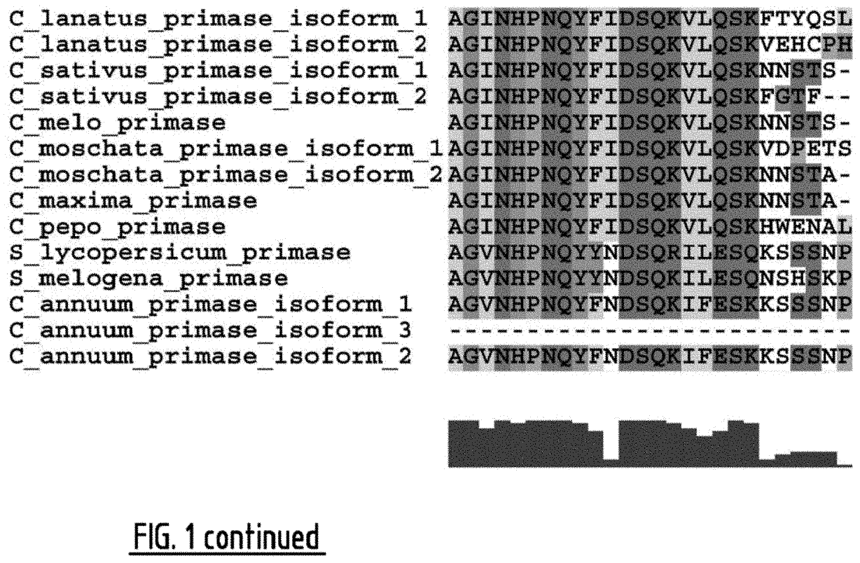

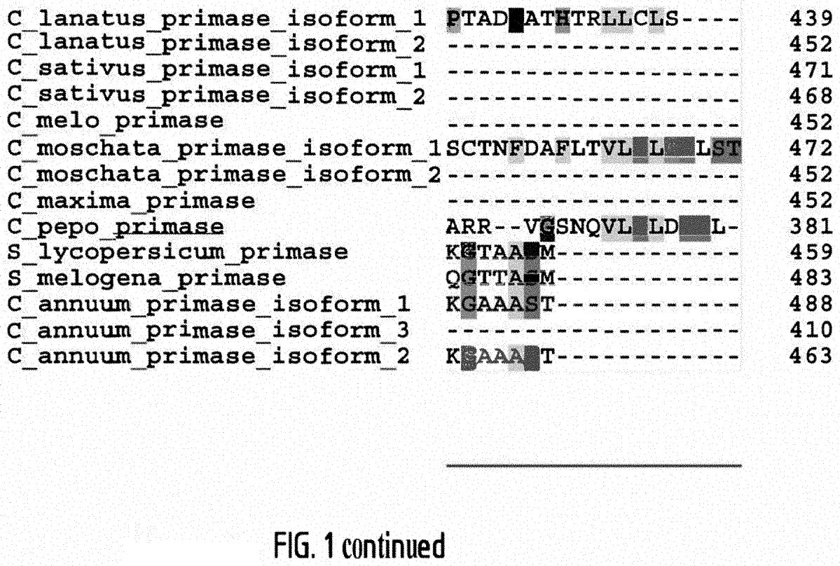

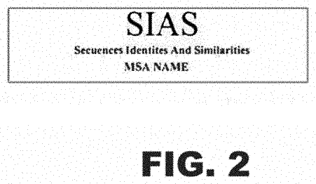

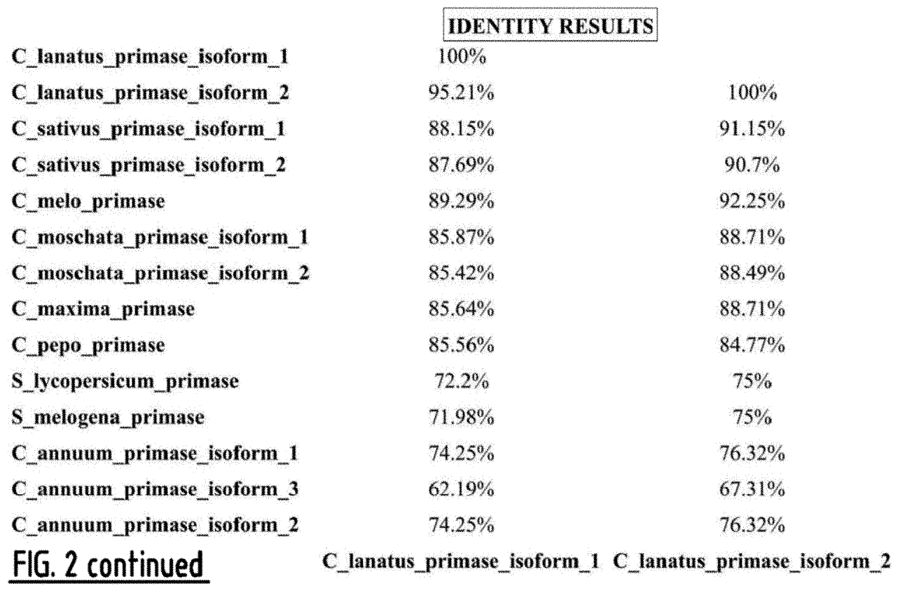

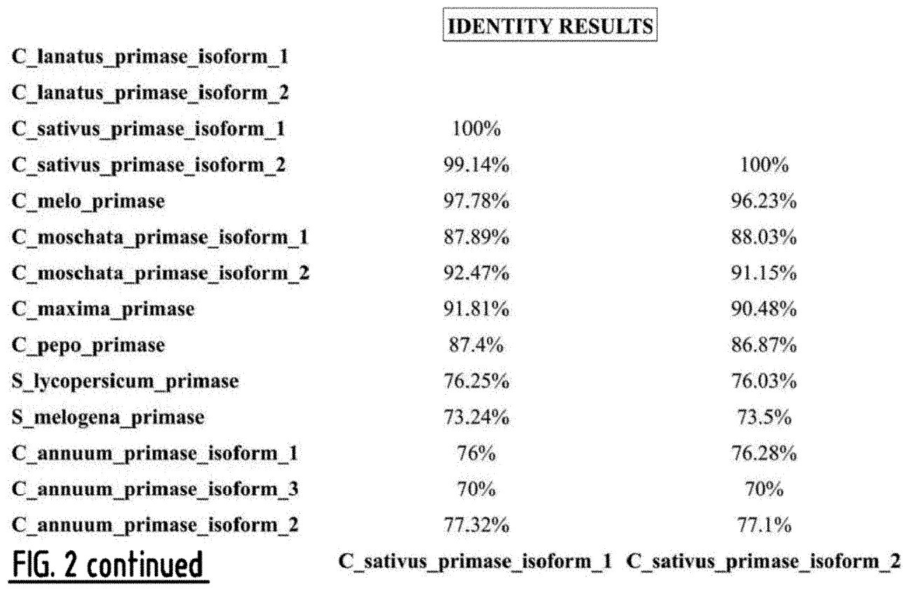

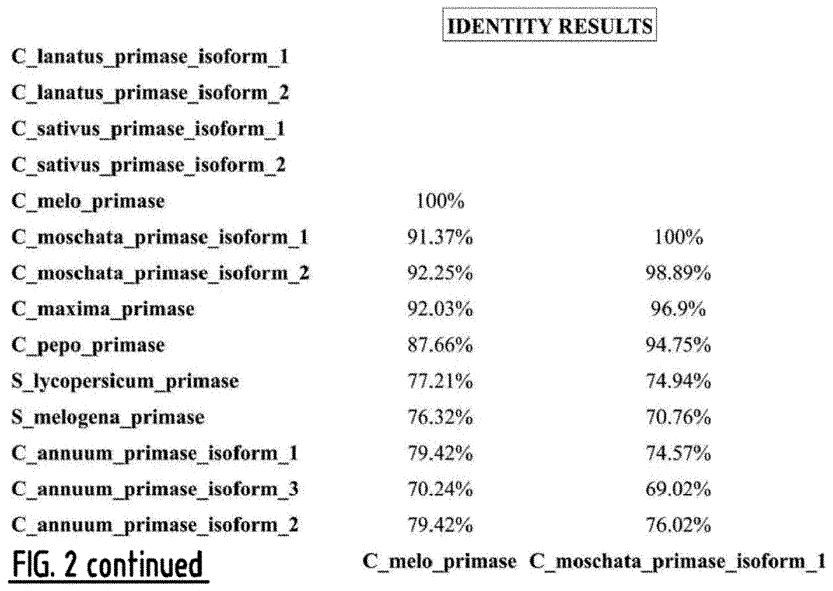

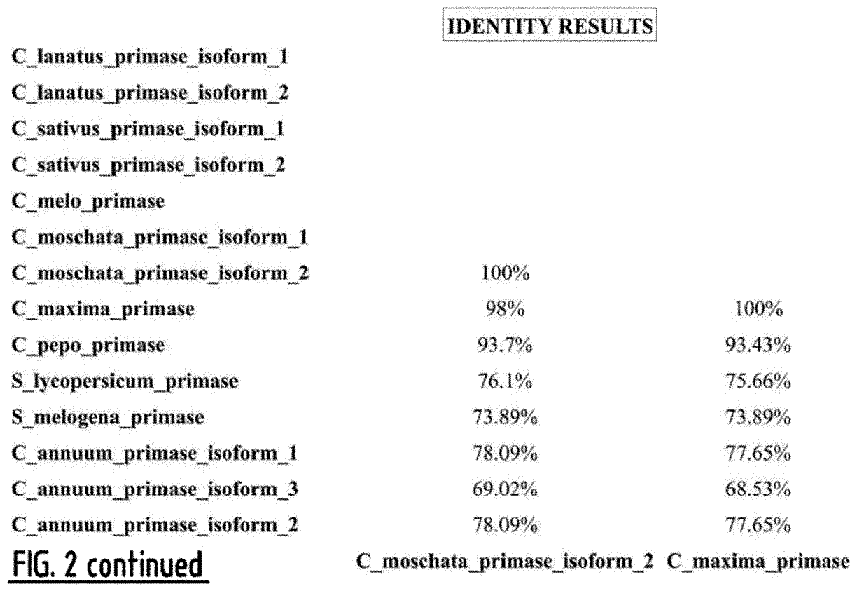

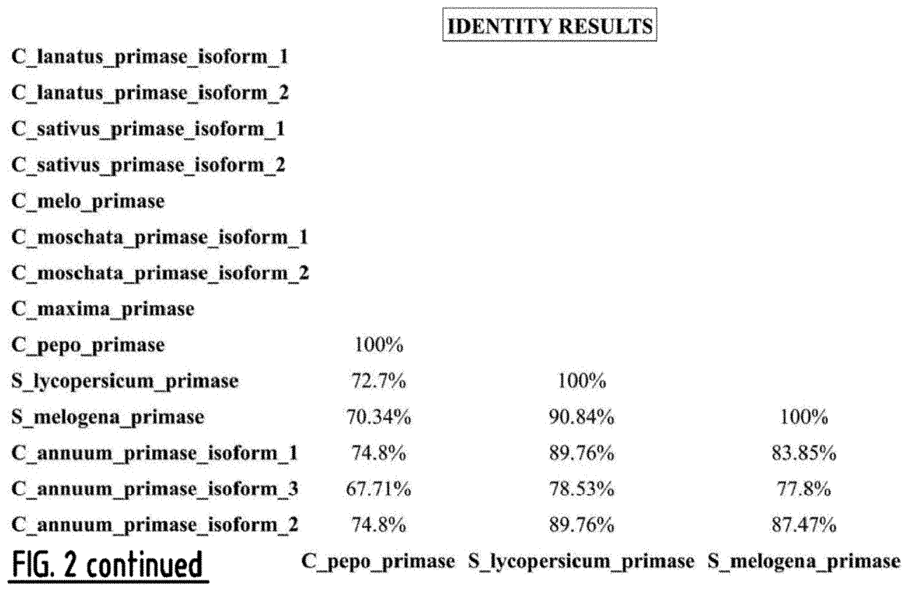

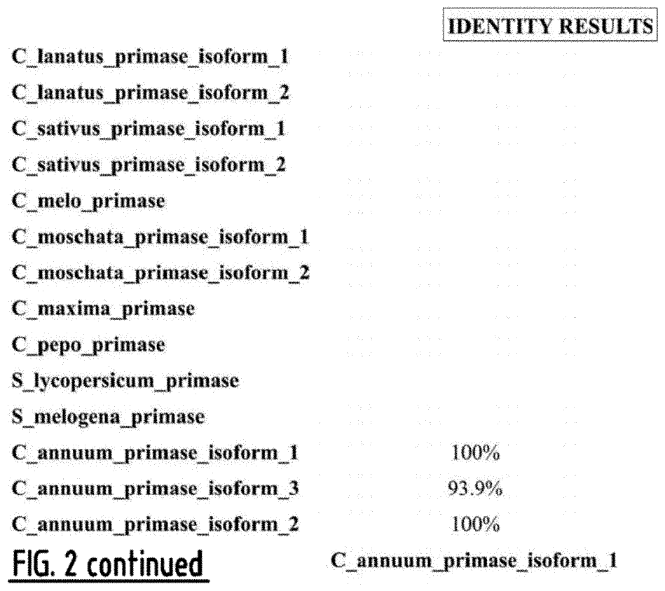

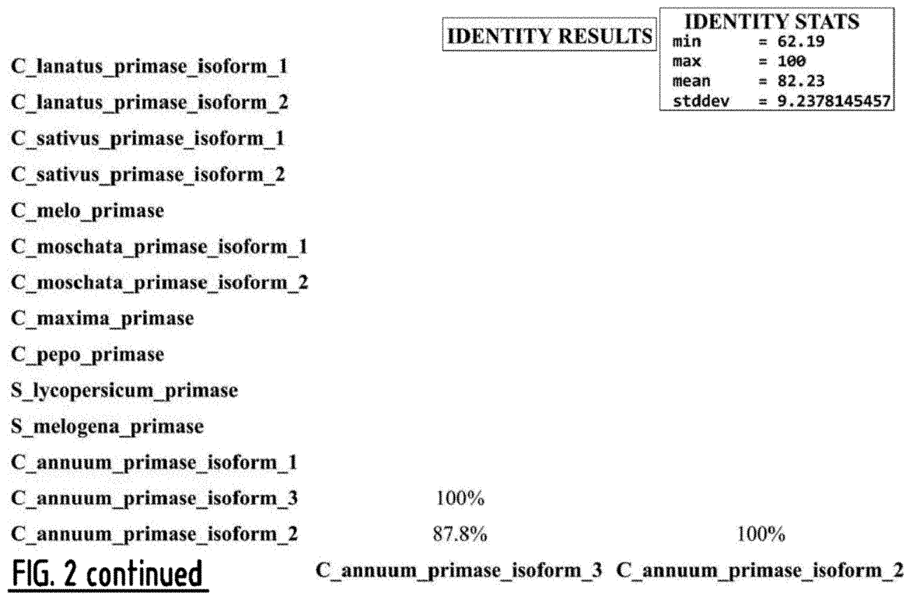

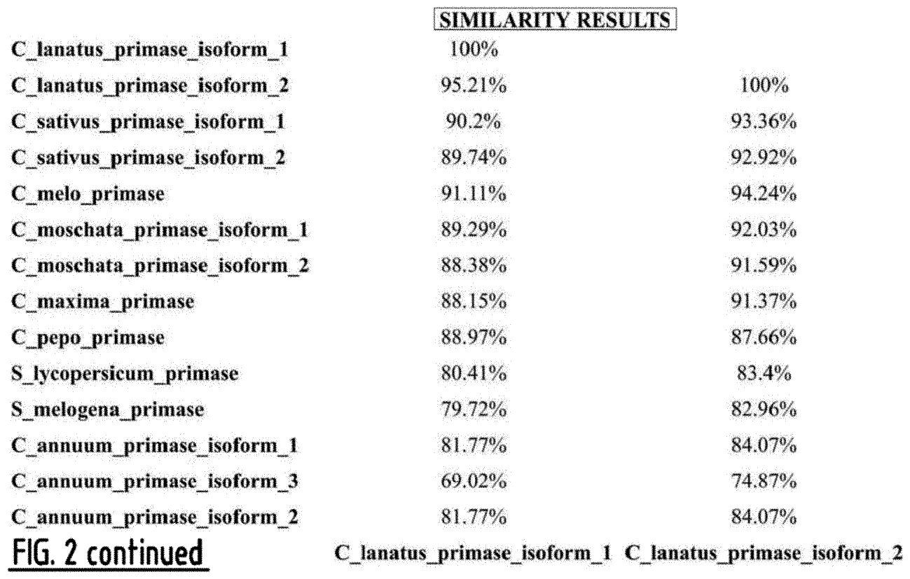

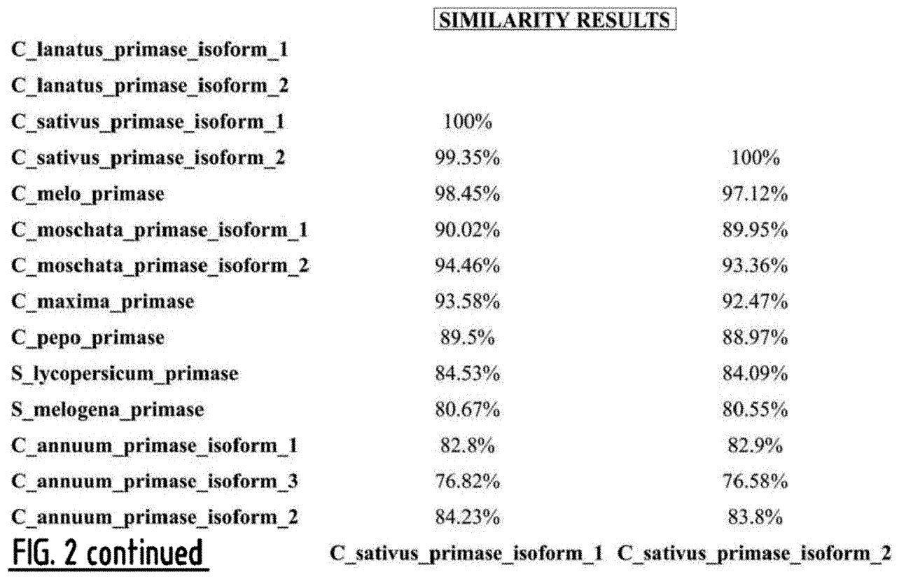

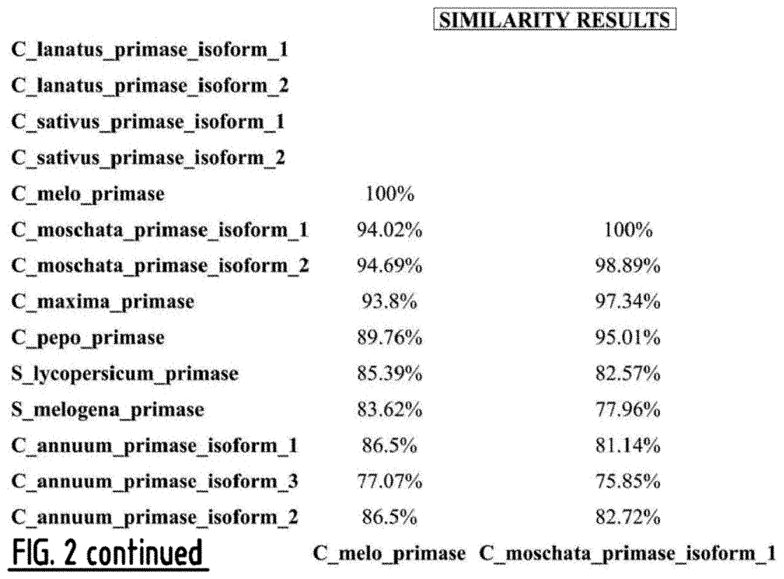

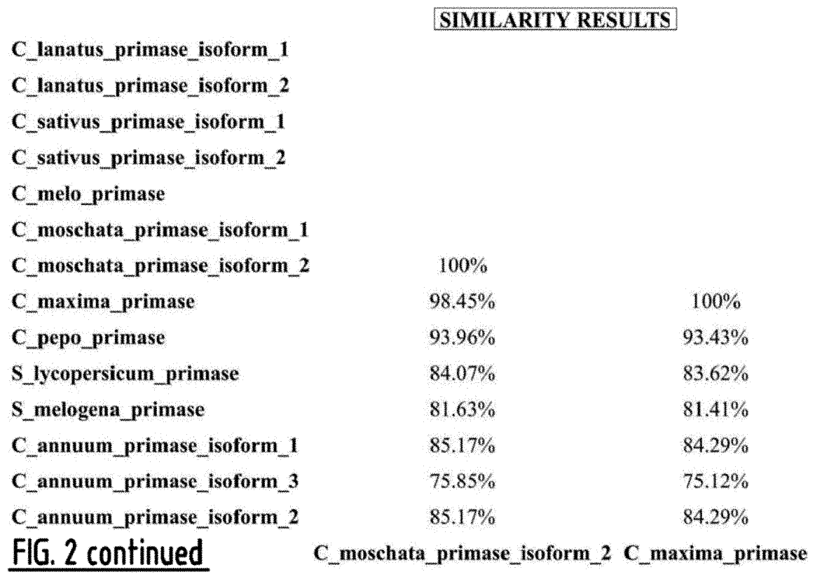

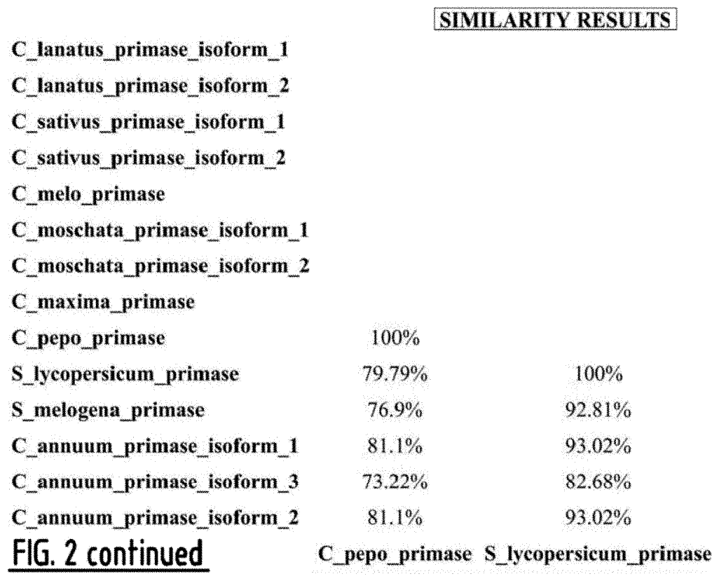

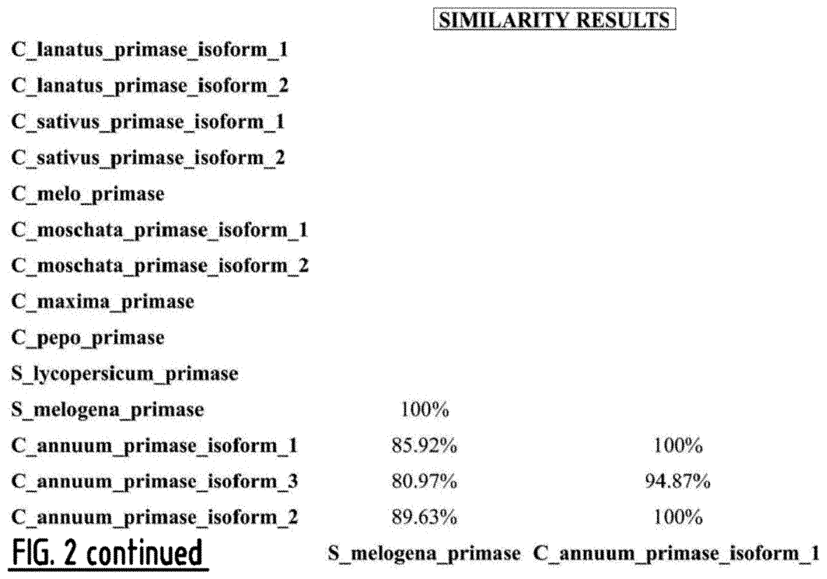

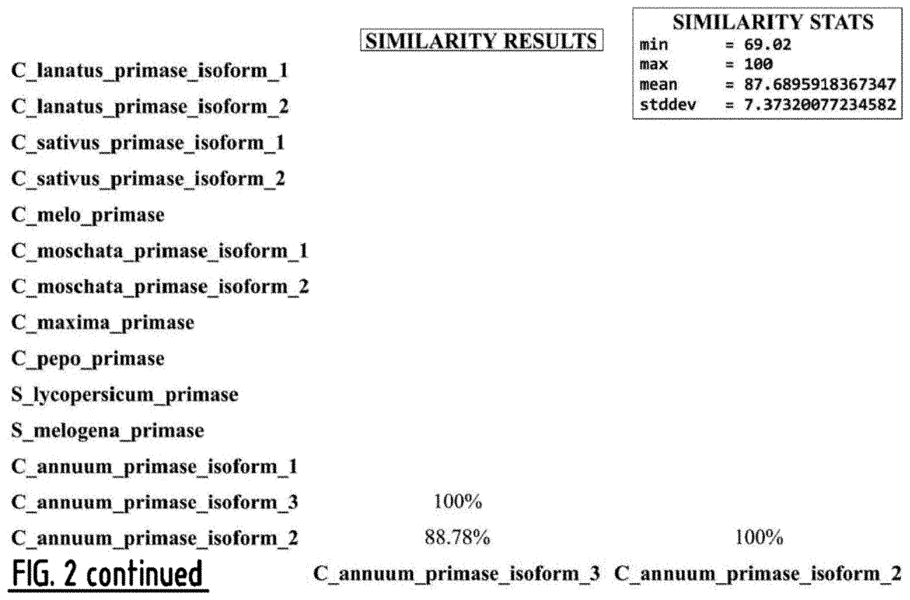

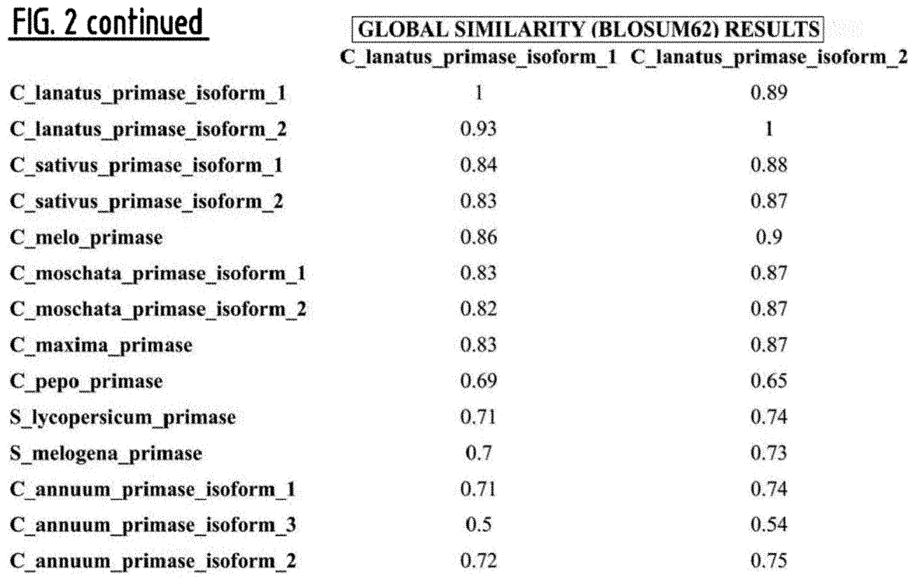

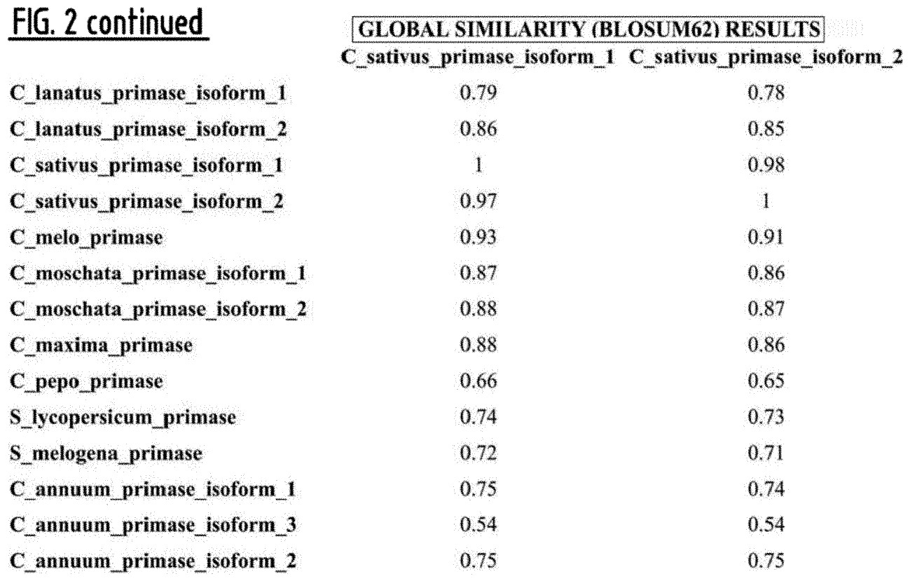

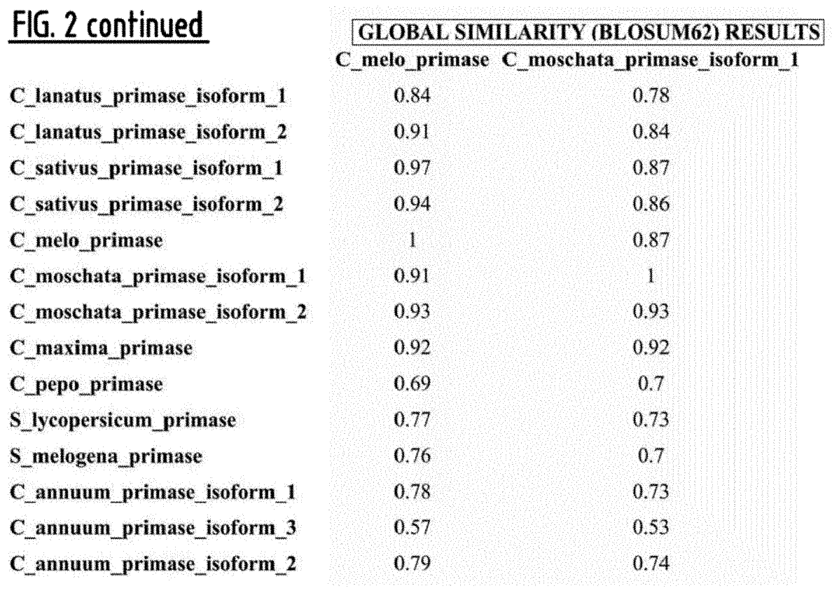

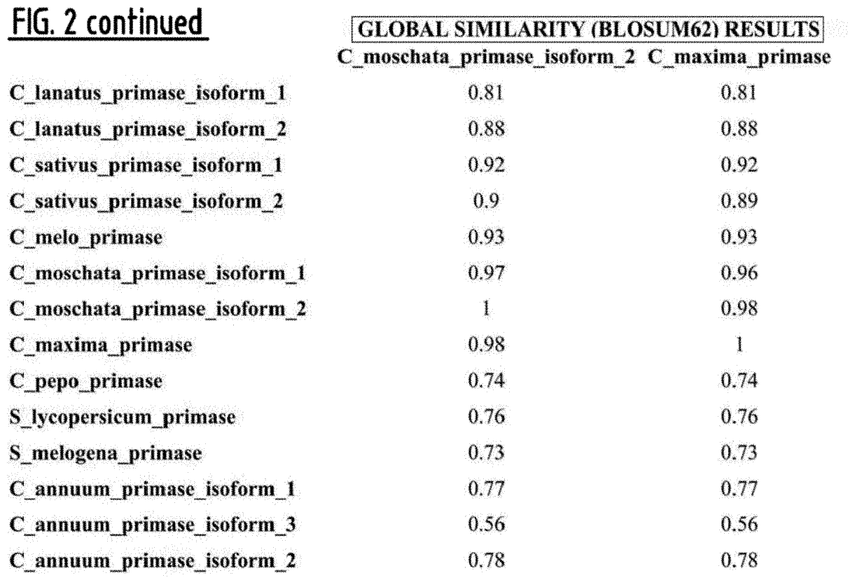

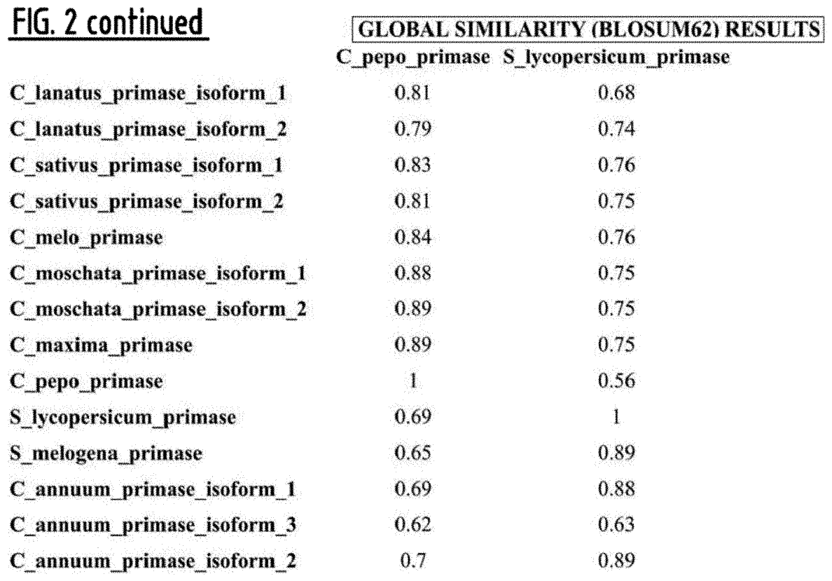

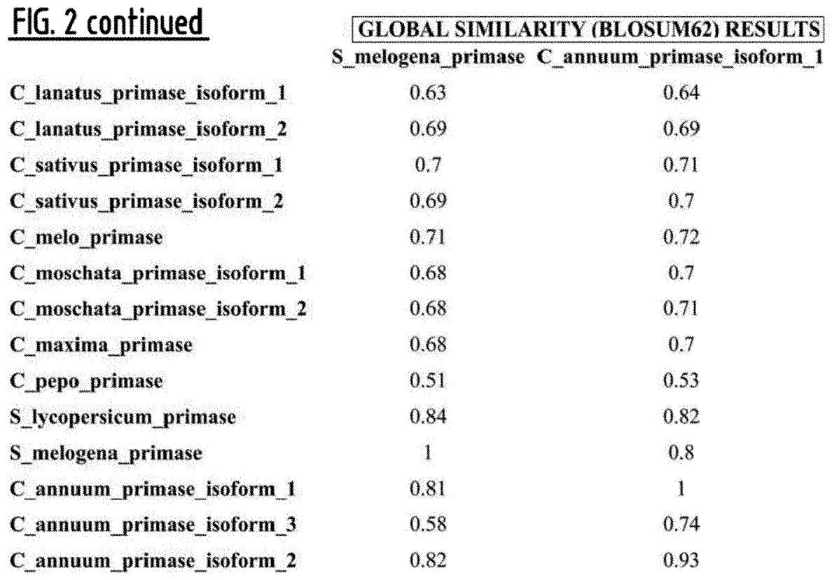

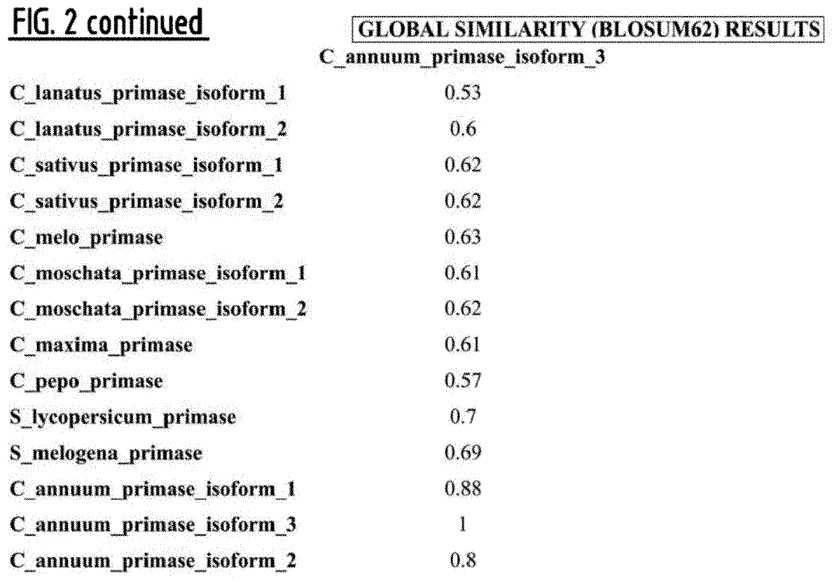

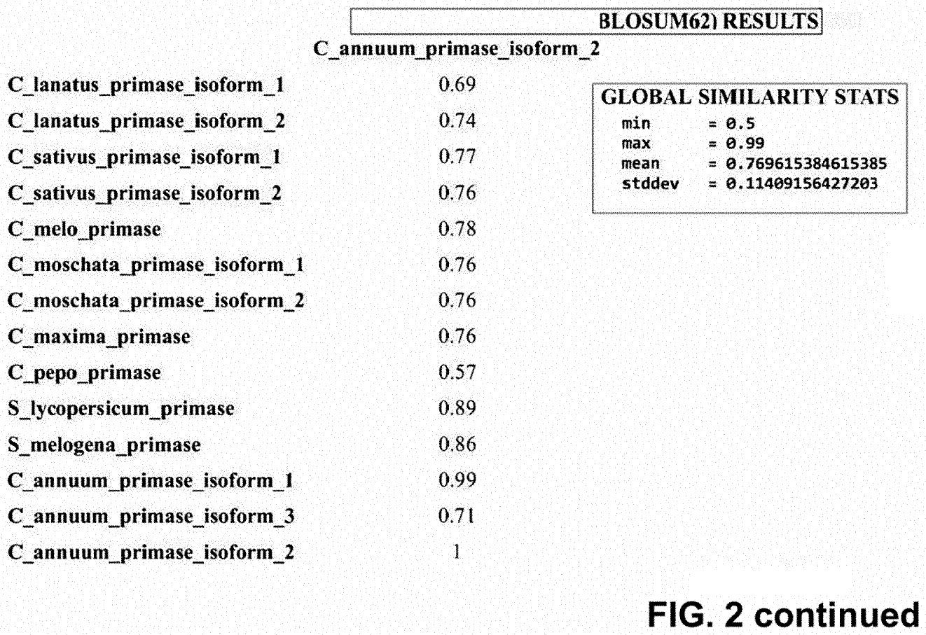

The PriL gene in other species are herein referred to as “orthologs” or “orthologous” PriL genes. Identification of PriL gene orthologs can be performed in different crop species, methods of which are known in the art. In the present research, orthologs of the PriL gene were identified using a Basic Local Alignment Search Tool (BLAST) to compare the C. moschata PriL DNA (SEQ ID NOS: 2 or 4) and protein sequence (SEQ ID NOS: 1 or 3) with the genome of other Cucurbitaceae and Solanaceae species. Using this method, 1-2 best hits per species were identified as candidate PriL orthologous genes. DNA and protein sequences of the PriL orthologs that were identified through this method are shown in Table 1. Multiple sequence alignments (MSA) of the predicted protein sequences confirmed that these were orthologous PriL gene (FIG. 1). Furthermore, the wild type PriL protein of C. pepo, C. melo, C. sativus, C. maxima, C. lanatus, S. lycopersicum, S. melongena and C. annuum has a high sequence identity and sequence similarity to the wild type PriL protein of C. moschata (FIG. 2).

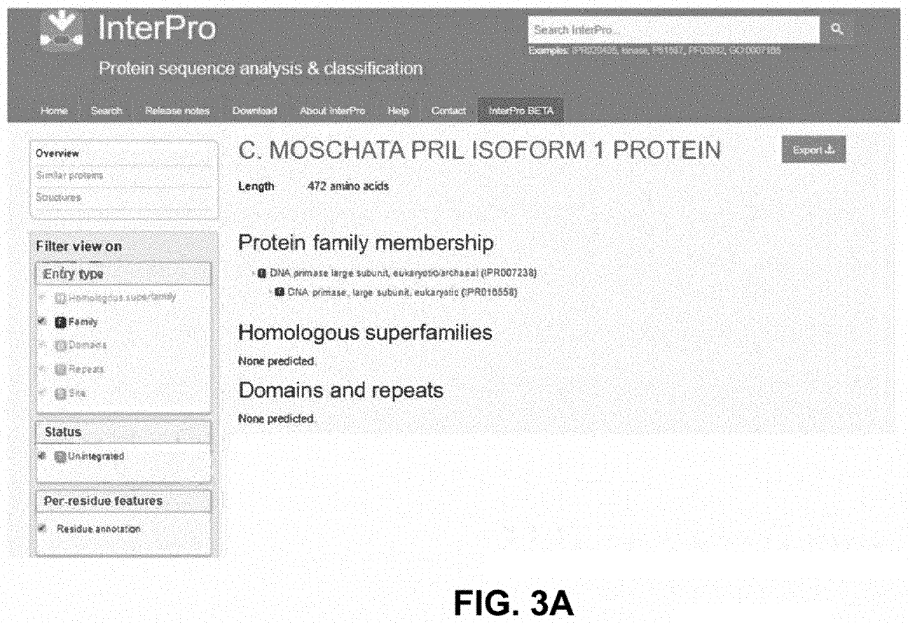

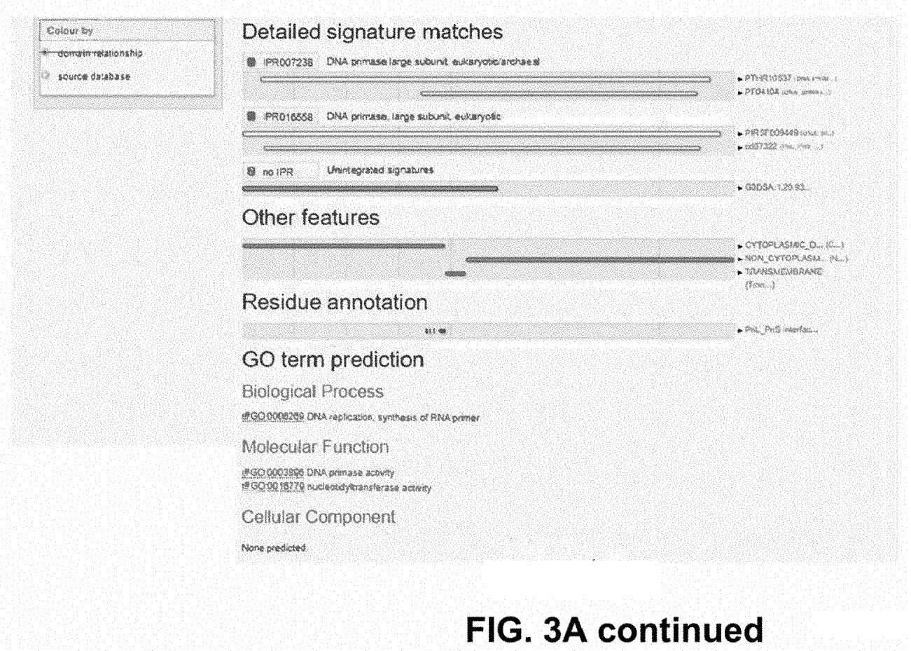

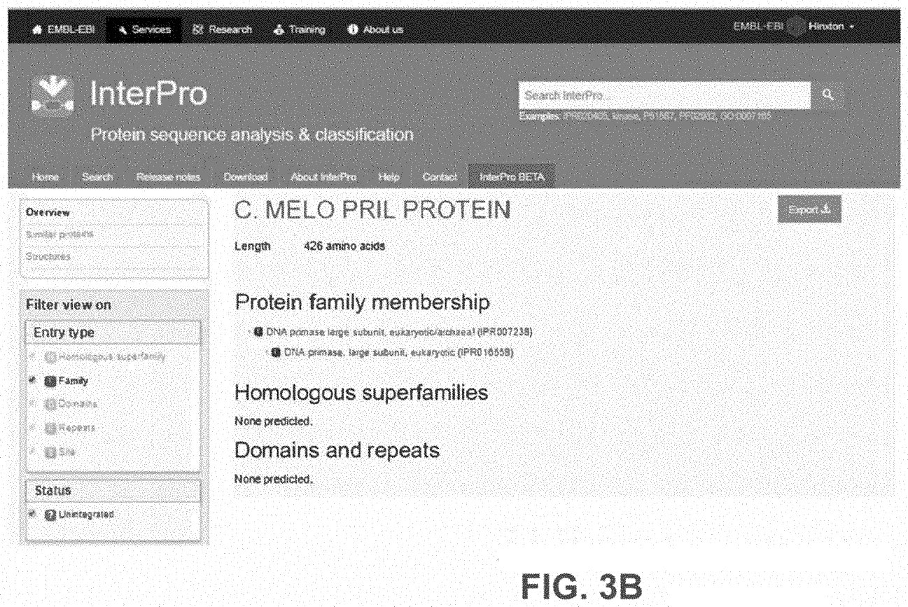

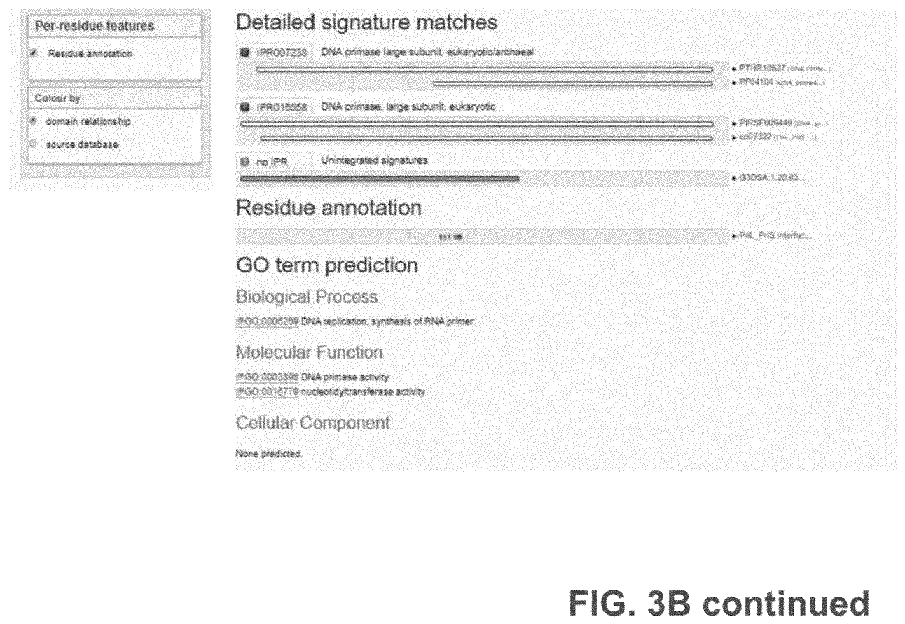

Functional analysis of the orthologous PriL protein sequences (InterProScan: Jones et al. (2014) Bioinformatics, 30(9): 1236-1240) revealed the protein's function, the protein families that the protein belongs to, and highly conserved domains that it contains. All PriL orthologs identified as part of this invention belong to the DNA primase large subunit, eukaryotic family (IPRO16558). PriL is predicted to have a role in the biological process of synthesizing short RNA primers from which DNA polymerases extend during DNA replication (Gene Ontology Accession: GO:0006269). It has DNA primase activity (Gene Ontology Accession: G00003896). For example, an InterProScan output for the functional analysis and classification of C. moschata and C. melo PriL ortholog proteins are shown in FIGS. 3A and 3B, respectively.

Once the DNA sequence of the orthologous PriL genes are known, this information may be used to modulate or modify the expression of said genes by methods herein described.

The invention relates to a modified PriL gene that encodes a modified protein which may comprise one or more modifications, which modified protein leads to Begomovirus resistance when present in a plant. This modified PriL gene is herein referred to as “the modified PriL gene of the invention”. The term “Begomovirus resistance”, as used herein, is to mean resistance to one or more Begomoviruses selected from the group consisting of ToLCNDV, ToLCPMV, CuLCV, MCLCV, MLCV, SqLCV, CuLCrV, TYLCV, WmCSV and WmCMoV. The term “resistance of the invention” or “trait of the invention” as used herein, is to mean resistance provided by the modified PriL gene of the invention to one or more Begomoviruses selected from the group consisting of ToLCNDV, ToLCPMV, CuLCV, MCLCV, MLCV, SqLCV, CuLCrV, TYLCV, WmCSV and WmCMoV, in particular, resistance to ToLCNDV. The terms “resistance of the invention”, “Begomovirus resistance”, “trait of the invention”, “trait” or “phenotypic trait”, can be used interchangeably when the underlying genetic basis for the resistance is provided by the modified PriL gene of the invention.

The resistance of the invention is controlled by modification(s) to the PriL gene, the inheritance of which is consistent with that of a monogenic recessive trait. The term “recessive trait” is to mean in the context of this application that the fully achievable trait is observed when the modified PriL gene is homozygously present in the genome such that both alleles of the PriL gene comprise the modification. When the modified PriL gene is heterozygously present in the genome, only one allele of the PriL gene is modified and therefore does not confer resistance to Begomoviruses. Since the inheritance of the trait is comparable to that of a monogenic trait, it is advantageous in that the trait can easily be incorporated into various plant types for a given plant species.

A “gene” in the context of this application comprises exonic sequences and regulatory sequences such as a promotor sequence, and if present also comprises intronic sequences. In this application the term “modification” or “modified” refers to a change in the sequence of the wild type PriL gene that results in an altered version of the wild type gene. A change or modification to the coding sequence (CDS) of the gene and/or the regulatory sequences of the gene in turn leads to a change in the amino acid sequence of the encoded protein and/or the transcription of the gene, such that the resultant modified PriL protein has a reduced level or reduced activity as compared to the wild type PriL protein. As used herein, “wild type” refers to the form of an organism, strain, gene, protein, characteristic or trait as it would occur in nature, and is in contrast to a mutated or modified form for example. In the context of the invention, the wild type PriL gene does not confer resistance to Begomoviruses. As used herein, the “coding sequence” is the portion of the gene's DNA composed of exons that code for protein. Modifications to the gene when recessive are to be present in the homozygous state to be visible. Some of the modifications described herein are recessive and thus only confer resistance in the homozygous form (i.e. presence of two alleles of the modified PriL gene), however, the heterozygous form in which there is a modification to a single allele of the PriL gene, also forms part of this invention.

The modified PriL gene of the invention encodes a modified protein which may comprise one or more modifications in the wild type protein sequence of SEQ ID NO: 1 or SEQ ID NO: 3. In another aspect of the invention, the modified PriL gene of the invention encodes a modified protein which may comprise one or more modifications in a protein sequence having at least 60% sequence identity to SEQ ID NO: 1 or SEQ ID NO: 3, preferably at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1 or SEQ ID NO: 3. The skilled person is familiar with methods for calculating sequence identity. Suitably sequence identity is calculated using the Sequence Identities and Similarities (SIAS) tool, which can be accessed at imed.med.ucm.es/Tools/sias.html. SIAS calculates pairwise sequence identity and similarity percentages between each pair of sequences from a multiple sequence alignment. Sequence identity is calculated using a method taking the gaps into account; sequence similarity is calculated based on grouping of amino acids having similar properties. For calculations, default settings for SIM percentage, similarity amino acid grouping, sequence length, normalized similarity score, matrix and gap penalties are used.

The invention thus relates to a modified PriL gene, the wild type of which encodes a protein as identified in SEQ ID NO: 1, SEQ ID NO: 3, SEQ ID NO: 7, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, or SEQ ID NO: 31.

In one embodiment the modified PriL gene of the invention is a nucleic acid, in particular a nucleic acid molecule, more in particular an isolated nucleic acid molecule.

The DNA sequence of a gene may be altered in a number of ways, and will have varying effects depending on where the modification(s) occur and whether they alter the function of the encoded protein. Examples of such modifications include amino acid substitutions, premature stop codons, insertions, deletions, or frameshift mutations.

An insertion changes the number of DNA bases in a gene by adding a piece of DNA. A deletion changes the number of DNA bases by removing one or a few base pairs, or even an entire gene or neighboring genes. These types of modifications may alter the function of the resulting protein.

Frame shift mutations are caused by insertion or deletion of one or more base pairs in a DNA sequence encoding a protein. When the number of inserted or deleted base pairs at a certain position is not a multiple of 3, the triplet codon encoding the individual amino acids of the protein sequence become shifted relative to the original open reading frame, and then the encoded protein sequence changes dramatically. Protein translation will result in an entirely different amino acid sequence than that of the originally encoded protein, and often a frameshift can lead to a premature stop codon in the open reading frame. The overall result is that the encoded protein no longer has the same biological function as the originally encoded protein.

An amino acid substitution in an encoded protein sequence arises when the mutation of one or more base pairs in the coding sequence results in an altered triplet codon, often encoding a different amino acid. Due to the redundancy of the genetic code not all point mutations lead to amino acid changes. Such mutations are termed “silent mutations”. Some amino acid changes are “conservative”, i.e. they lead to the replacement of one amino acid by another amino acid with comparable properties, such that the mutation is unlikely to dramatically change the folding of the mature protein, or influence its function. Conservative amino acid substitutions may be made on the basis of chemical properties, for example similarity in polarity, charge, solubility, hydrophobicity, hydrophilicity or amphipathic nature of the residues, in which case the resulting protein may still function normally. Other amino acid changes are non-silent, non-conservative amino acid changes in domains that play a role in substrate recognition, the active site of enzymes, interaction domains or in major structural domains (such as transmembrane helices) may partly or completely destroy the functionality of an encoded protein, without thereby necessarily affecting the expression level of the encoding gene. As used herein, a “non-conservative amino acid change” occurs when there is an amino acid substitution at a well conserved or invariant position that is essential for the structure and/or function of the protein, or substitutions with amino acids that do not share conserved chemical properties (e.g. hydrophobic vs. charged vs. polar), which may lead to detrimental stability, functionality and/or structural effects of the encoded protein.

Sequencing of C. moschata and C. melo plants that were identified to be resistant to ToLCNDV revealed that these resistant C. moschata and C. melo plants comprise modifications to their PriL gene. The identified modifications lead to amino acid substitutions in the encoded PriL protein, which without being bound to theory, are not expected to severely change the native protein function of the PriL subunit. It is however expected that these amino acid substitutions have an effect on the ability of the Begomovirus to utilize host Primase for viral replication.

The modified PriL gene encodes a modified protein which may comprise one or more modifications. In one embodiment, the one or more modifications in the modified PriL protein, the wild type protein sequence as identified in SEQ ID NO: 1, SEQ ID NO: 3, SEQ ID NO: 7, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, or SEQ ID NO: 31, is an amino acid substitution.

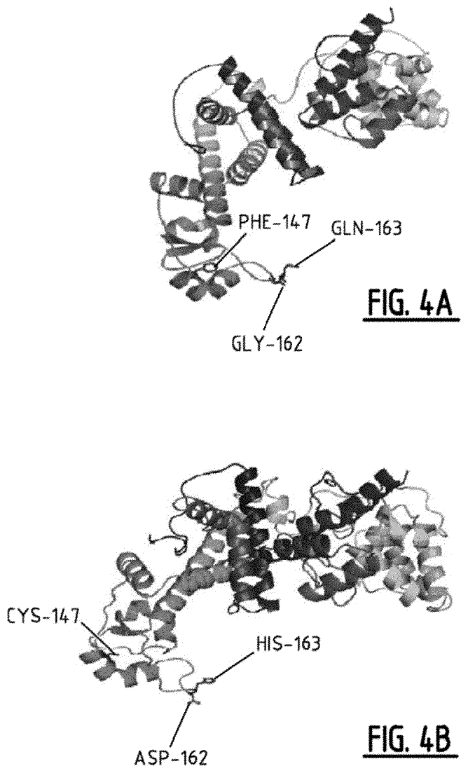

The amino acid substitution of phenylalanine (F) to cysteine (C) at a position corresponding to position 147 according to SEQ ID NO: 5, the amino acid substitution of glycine (G) to aspartic acid (D) at position corresponding to position 162 according to SEQ ID NO: 5, and the amino acid substitution of glutamine (Q) to histidine (H) at a position corresponding to position 163 according to SEQ ID NO: 5, were identified in the present research and are located at the exposed surface of the protein (FIGS. 4A and 4B). In the present research, the NCBI Amino Acid Explorer (https://www.ncbi.nlm.nih.gov/Class/Structure/aa/aa_explorer.cgi) was used as a tool in understanding the propensity of exchanging one amino acid for another. Mutation matrices such as BLOSUM62, employed by the NCBI Amino Acid Explorer, can be used for example to indicate favorable and unfavorable amino acid changes, the expected effect of amino acid substitutions on protein structure, and the changes in charge that may occur with substituting one amino acid for another. The phenylalanine (F) to cysteine (C) at position 147 leads to the modification of a nonpolar, aromatic amino acid with a polar aliphatic amino acid, respectively. This F147C modification is expected to reduce the flexibility of the side chain structure of the C. moschata PriL protein, while the backbone structure of the protein remains unaffected. The amino acid substitution of glycine (G) to aspartic acid (D) at position 162 leads to the modification of a nonpolar amino acid with a negatively charged amino acid, respectively. This G162D modification is expected to provide flexibility to the side chain structure of the C. moschata PriL protein, since the structure of a glycine molecule provides no independent side chain motion. The backbone structure of the protein remains unaffected. The amino acid substitution of glutamine (Q) to histidine (H) at position 163 leads to the modification of a polar, aliphatic amino acid with a positively charged aromatic amino acid, respectively. This Q163H modification is expected to reduce the flexibility of the side chain structure of the C. moschata PriL protein, while the backbone structure of the protein remains unaffected. The identified amino acid substitutions are all located within the cytoplasmic domain of the C. moschata PriL protein. These amino acid substitutions have an effect on the ability of the Begomovirus to interact with host Primase for viral replication.

In one embodiment, the invention relates to a modified PriL gene which encodes a modified protein that may comprise a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5. The modified protein is capable of imparting Begomovirus resistance, in particular ToLCNDV resistance, when present in a C. moschata or a C. pepo plant. More in particular, the modified protein is capable of imparting ToLCNDV resistance when present in a C. moschata or a C. pepo plant.

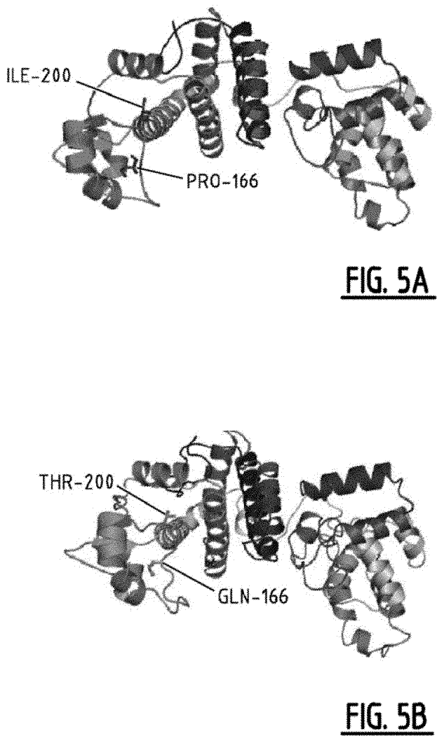

The amino acid substitution of tyrosine (Y) to histidine (H) at a position corresponding to position 4 according to SEQ ID NO: 9, the amino acid substitution of proline (P) to glutamine (Q) at a position corresponding to position 166 according to SEQ ID NO: 9, and the amino acid substitution of isoleucine (I) to threonine (T) at a position corresponding to position 200 according to SEQ ID NO: 9, were identified in the present research and are located at the exposed surface of the protein (FIGS. 5A and 5B). The amino acid substitution of tyrosine (Y) to histidine (H) at position 4 leads to the modification of a polar amino acid with a positively charged amino acid. This substitution while not expected to change the flexibility of the side chain structure of the C. melo PriL protein, it provides a reduction in the hydrophobicity of the side chain. The amino acid substitution of proline (P) to glutamine (Q) at position 166 leads to the modification of a nonpolar amino acid with a polar amino acid, respectively. This is a fairly rare substitution which increases the side chain flexibility of the C. melo PriL protein such that it becomes highly flexible, while the backbone structure of the protein remains unaffected. The amino acid substitution isoleucine (I) to threonine (T) at position 200 leads to the modification of a nonpolar amino acid with a polar amino acid, respectively. This I200T modification is expected to reduce the flexibility of the side chain structure of the C. melo PriL protein, while the backbone structure of the protein remains unaffected. The identified amino acid substitutions are located within the cytoplasmic domain of the C. melo PriL protein, with the exception of I200T, which is located within a transmembrane domain. These amino acid substitutions have an effect on the ability of the Begomovirus to interact with host Primase for viral replication.

In another embodiment, the invention relates to a modified PriL gene which encodes a modified protein that may comprise a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9. The modified protein provides Begomovirus resistance, in particular ToLCNDV resistance, when present in a C. melo plant More in particular, the modified protein is capable of imparting ToLCNDV resistance when present in a C. melo plant.

In a further embodiment of the invention, the invention relates to a modified PriL gene which encodes a modified protein that may comprise a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5, and/or wherein the modified gene encodes a modified protein that may comprise a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9. The modified protein is capable of imparting resistance to one or more of the Begomoviruses selected from the group consisting of ToLCNDV, ToLCPMV, CuLCV, MCLCV, MLCV, SqLCV, CuLCrV, TYLCV, WmCSV and WmCMoV, when present in a C. moschata, C. pepo, C. melo, C. sativus, C. maxima, C. lanatus, S. lycopersicum, S. melongena or C. annuum plant.

Mutations in the regulatory sequences such as the promotor sequence of a gene may lead to a complete lack of transcription of the gene (e.g. subsequently resulting in a complete absence of the encoded protein), or to a significantly decreased and biologically inadequate level of transcription (e.g. subsequently resulting in a reduced level of the encoded protein). Mutations in splice sites may perturb the biological function of the encoded protein, because if a splice site is destroyed by a mutation the amino acid sequence encoded in the mature mRNA transcribed from the gene will not be correct, and it may easily contain frame shifts and/or premature stop codons. In either case the protein sequence translated from such an mRNA will not be identical to the wild type protein sequence, leading to serious consequences.

As a result of the one or modifications, the encoded modified protein of the invention imparts resistance to one or more of the following Begomoviruses selected from ToLCNDV and/or ToLCPMV and/or CuLCV and/or MCLCV and/or MLCV and/or SqLCV and/or CuLCrV and/or TYLCV and/or WmCSV and/or WmCMoV, in particular, resistance to ToLCNDV in a plant in which the gene encoding the modified protein is homozygously present.

The presence of a modified PriL gene or a modified PriL protein leading to Begomovirus resistance may be detected using routine methods known to the skilled person such as RT-PCR, PCR, antibody-based assays, sequencing and genotyping assays, or combinations thereof. Such methods may be used to determine for example, a reduction of the expression of the wild type PriL gene, a reduction of the wild type PriL protein, the presence of a modified mRNA, cDNA or genomic DNA encoding a modified PriL protein, or the presence of a modified PriL protein, in plant material or plant parts, or DNA or RNA or protein derived therefrom.

Modifications or mutations of the PriL gene can be introduced randomly by means of one or more chemical compounds, such as ethyl methane sulphonate (EMS), nitrosomethylurea, hydroxylamine, proflavine, N-methly-N-nitrosoguanidine, N-ethyl-N-nitrosourea, N-methyl-N-nitro-nitrosoguanidine, diethyl sulphate, ethylene imine, sodium azide, formaline, urethane, phenol and ethylene oxide, and/or by physical means, such as UV-irradiation, fast neutron exposure, X-rays, gamma irradiation, and/or by insertion of genetic elements, such as transposons, T-DNA, retroviral elements.

Mutagenesis also may comprise the more specific, targeted introduction of at least one modification by means of homologous recombination, oligonucleotide-based mutation introduction, zinc-finger nucleases (ZFN), transcription activator-like effector nucleases (TALENs) or Clustered Regularly Interspaced Short Palindromic Repeat (CRISPR) systems.

Modifying a wild type PriL gene could also comprise the step of targeted genome editing, wherein the sequence of a wild type PriL gene is modified, or wherein a wild type PriL gene is replaced by another PriL gene that is optionally modified. This can be achieved by means of any method known in the art for modifying DNA in the genome of a plant, or by means of methods for gene replacement. Such methods include genome editing techniques and homologous recombination.

Homologous recombination allows the targeted insertion of a nucleic acid construct into a genome, and the targeting is based on the presence of unique sequences that flank the targeted integration site. For example, the wild type locus of a PriL gene could be replaced by a nucleic acid construct which may comprise a modified PriL gene.

Modifying a wild type PriL gene can involve inducing double strand breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered to make double-strand breaks at specific recognition sequences in the genome of a plant, another organism, or a host cell.

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, Fok I. The unique, modular TAL effector DNA binding domain allows for the design of proteins with potentially any given DNA recognition specificity. Thus, the DNA binding domains of the TAL effector nucleases can be engineered to recognise specific DNA target sites and thus, used to make double-strand breaks at desired target sequences.

ZFNs can be used to make double-strand breaks at specific recognition sequences in the genome of a plant for gene modification or gene replacement through homologous recombination. The Zinc Finger Nuclease (ZFN) is a fusion protein which may comprise the part of the Fok I restriction endonuclease protein responsible for DNA cleavage and a zinc finger protein which recognizes specific, designed genomic sequences and cleaves the double-stranded DNA at those sequences, thereby producing free DNA ends (Urnov et al, 2010, Nat. Rev. Genet. 11:636-46; Carroll, 2011, Genetics 188:773-82).

The CRISPR/Cas nuclease system can also be used to make double-strand breaks at specific recognition sequences in the genome of a plant for gene modification or gene replacement through homologous recombination. The CRISPR/Cas nuclease system is an RNA-guided DNA endonuclease system performing sequence-specific double-stranded breaks in a DNA segment homologous to the designed RNA. It is possible to design the specificity of the sequence (Jinek et al, 2012, Science 337: 816-821; Cho et al, 2013, Nat. Biotechnol. 31:230-232; Cong et al, 2013, Science 339:819-823; Mali et al., 2013, Science 339:823-826; Feng et al, 2013, Cell Res. 23:1229-1232). Cas9 is an RNA-guided endonuclease that has the capacity to create double-stranded breaks in DNA in vitro and in vivo, also in eukaryotic cells. It is part of an RNA-mediated adaptive defence system known as Clustered Regularly Interspaced Short Palindromic Repeats (CRISPR) in bacteria and archaea. Cas9 gets sequence-specificity when it associates with a guide RNA molecule, which can target sequences present in an organism's DNA based on their sequence. Cas9 requires the presence of a Protospacer Adjacent Motif (PAM) immediately following the DNA sequence that is targeted by the guide RNA. The Cas9 enzyme has been first isolated from Streptococcus pyogenes (SpCas9), but functional homologues from many other bacterial species have been reported, such as Neisseria meningitides, Treponema denticola, Streptococcus thermophilus, Francisella novicida, Staphylococcus aureus, etcetera. For SpCas9, the PAM sequence is 5′-NGG-3′, whereas various Cas9 proteins from other bacteria have been shown to recognise different PAM sequences. In nature, the guide RNA is a duplex between crRNA and tracrRNA, but a single guide RNA (sgRNA) molecule which may comprise both crRNA and tracrRNA has been shown to work equally well (Jinek et al, 2012, Science 337: 816-821). The advantage of using an sgRNA is that it reduces the complexity of the CRISPR-Cas9 system down to two components, instead of three. For use in an experimental setup (in vitro or in vivo) this is an important simplification.

An alternative for Cas9 is, for example, Cpf1, which does not need a tracrRNA to function, which recognises a different PAM sequence, and which creates sticky end cuts in the DNA, whereas Cas9 creates blunt ends.

On the one hand, genetic modification techniques can be applied to express a site-specific nuclease, such as an RNA-guided endonuclease and/or guide RNAs, in eukaryotic cells. One or more DNA constructs encoding an RNA-guided endonuclease and at least one guide RNA can be introduced into a cell or organism by means of stable transformation (wherein the DNA construct is integrated into the genome) or by means of transient expression (wherein the DNA construct is not integrated into the genome, but it expresses an RNA-guided endonuclease and at least one guide RNA in a transient manner). This approach requires the use of a transformation vector and a suitable promoter for expression in said cell or organism. Organisms into which foreign DNA has been introduced are considered to be Genetically Modified Organisms (GMOs), and the same applies to cells derived therefrom and to offspring of these organisms. In important parts of the worldwide food market, transgenic food is not allowed for human consumption, and not appreciated by the public. There is however also an alternative, “DNA-free” delivery method of CRISPR-Cas components into intact plants, that does not involve the introduction of DNA constructs into the cell or organism.

For example, introducing the mRNA encoding Cas9 into a cell or organism has been described, after in vitro transcription of said mRNA from a DNA construct encoding an RNA-guided endonuclease, together with at least one guide RNA. This approach does not require the use of a transformation vector and a suitable promoter for expression in said cell or organism.

Another known approach is the in vitro assembly of ribonucleoprotein (RNP) complexes, which may comprise an RNA-guided endonuclease protein (for example Cas9) and at least one guide RNA, and subsequently introducing the RNP complex into a cell or organism. In animals and animal cell and tissue cultures, RNP complexes have been introduced by means of, for example, injection, electroporation, nanoparticles, vesicles, and with the help of cell-penetrating peptides. In plants, the use of RNPs has been demonstrated in protoplasts, for example with polyethylene glycol (PEG) transfection (Woo et al, 2015, Nat. Biotech. 33: 1162-1164). After said modification of a genomic sequence has taken place, the protoplasts or cells can be used to produce plants that harbour said modification in their genome, using any plant regeneration method known in the art (such as in vitro tissue culture).

Breaking DNA using site specific nucleases, such as, for example, those described herein above, can increase the rate of homologous recombination in the region of the breakage. Thus, coupling of such effectors as described above with nucleases enables the generation of targeted changes in genomes which include additions, deletions and other modifications.

A skilled person is able to visually assess symptoms and relate the symptoms or a lack thereof, to whether a plant is resistant or susceptible to Begomoviruses. For example, ToLCNDV symptoms on C. moschata and C. pepo plants are described in Table 2 and ToLCNDV symptoms on C. melo plants are described in Table 4, when measured under the conditions as described in Example 1. In general, ToLCNDV resistant plants exhibit no ToLCNDV symptoms, non-specific yellowing, or minimal virus symptoms such as some yellowing spots on older leaves and less than 25% of the plant surface is affected, and an otherwise healthy plant character.

The modified PriL gene of the invention as herein described, encodes a modified protein which may comprise one or more modifications in the wild type PriL protein sequence as identified in SEQ ID NO: 1, SEQ ID NO: 3, SEQ ID NO: 7, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, or SEQ ID NO: 31. The invention thus also relates to a modified PriL protein. This modified PriL protein is also referred to herein as the “modified protein of the invention” and as a result of the one or more modifications, imparts a plant with Begomovirus resistance, in particular, to one or more of the following Begomoviruses selected from the group consisting of ToLCNDV, ToLCPMV, CuLCV, MCLCV, MLCV, SqLCV, CuLCrV, TYLCV, WmCSV and WmCMoV when the modified PriL gene encoding the modified PriL protein is homozygously present in the genome of the plant.

The invention also relates to a plant which may comprise in its genome the modified PriL gene of the invention. This plant is referred to herein as a “plant of the invention”. A plant of the invention can comprise the modified PriL gene of the invention heterozygously, in which case the plant is not resistant to Begomoviruses but is useful for transferring the modified PriL gene of the invention to another plant. A plant of the invention can also comprise the modified PriL gene of the invention homozygously. A plant of the invention can be a plant of an inbred line, a hybrid, a doubled haploid or a plant of a segregating population. Preferably the plant of the invention is non-transgenic.

As used herein, a plant of an inbred line is a plant of a population of plants that is the result of three or more rounds of selfing, or backcrossing; or which plant is a doubled haploid. An inbred line may e.g. be a parent line used for the production of a commercial hybrid.

As used herein, a hybrid plant is a plant which is the result of a cross between two different plants having different genotypes. More in particular, a hybrid plant is the result of a cross between plants of two different inbred lines, such a hybrid plant may e.g. be a plant of an F₁ hybrid variety.

A plant of the invention is a plant selected from any one of the species C. moschata, C. pepo, C. melo, C. sativus, C. maxima, C. lanatus, S. lycopersicum, S. melongena or C. annuum, in particular a C. moschata, C. pepo or C. melo plant.

In certain embodiments of the invention, the modified PriL gene of the invention has been introgressed from wild, uncultivated germplasm into the germplasm of cultivated, agronomically useful and preferably, elite plants. After one or more rounds of selfing and/or backcrossing to one of the parents, and selection for Begomovirus resistance, this results in a Begomovirus resistant cultivated, elite plant which may comprise the modified PriL gene of the invention, but lacks the undesirable horticultural qualities of the wild material such as small or malformed fruit shape, irregular plant architecture, low yield etc. Begomovirus resistant elite plants which may comprise the modified PriL gene of the invention are thus also plants of the invention.

In a further embodiment, the plant of the invention is an agronomically elite plant, preferably an agronomically elite plant selected from any one of the species C. moschata, C. pepo, C. melo, C. sativus, C. maxima, C. lanatus, S. lycopersicum, S. melongena or C. annuum, preferably an agronomically elite C. moschata, or C. pepo plant or a C. melo plant.

In the context of this invention, an agronomically elite plant is a plant having a genotype that, as a result of human intervention, may comprise an accumulation of distinguishable and desirable agronomic traits which allow a producer to harvest a product of commercial significance, preferably the agronomically elite plant of the invention is a plant of an inbred line or a hybrid.

In other embodiments of the invention, a plant selected from any one of the species C. moschata, C. pepo, C. melo, C. sativus, C. maxima, C. lanatus, S. lycopersicum, S. melongena or C. annuum bearing the modified PriL gene of the invention can be used as the donor plant to produce more plants of any one of the species C. moschata, C. pepo, C. melo, C. sativus, C. maxima, C. lanatus, S. lycopersicum, S. melongena or C. annuum. Where biologically possible, interspecific crosses between species can also be used to transfer the modified PriL gene of the invention. In the present invention for example, interspecific crosses were made to in order to produce ToLCNDV resistant C. pepo plants which may comprise a modified PriL gene derived from C. moschata (See Example 2).

In one embodiment, the plant of the invention is a C. moschata or C. pepo plant, which may comprise in its genome a modified PriL gene of the invention, wherein the modified PriL gene encodes a modified protein which may comprise one or more modifications in the wild type protein sequence of SEQ ID NO: 1, SEQ ID NO: 3, or SEQ ID NO: 11. This plant is also referred to herein as a “C. moschata or C. pepo plant of the invention”. When the C. moschata or C. pepo plant of the invention may comprise the modified PriL gene of the invention homozygously, the plant is resistant to one or more of the following Begomoviruses selected from ToLCNDV and/or ToLCPMV and/or CuLCV and/or MCLCV and/or MLCV and/or SqLCV and/or CuLCrV and/or TYLCV and/or WmCSV and/or WmCMoV as a result of the presence of the modified protein.

In one embodiment, the C. moschata or C. pepo plant of the invention may comprise in its genome a modified PriL gene, wherein the modified PriL gene encodes a modified protein which may comprise a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5. The C. moschata or C. pepo plant of the invention is resistant to ToLCNDV.

In another embodiment, the modified PriL gene of the invention encodes a modified protein that may comprise a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5 and the modified PriL gene is homozygously present in a plant grown from a seed deposited under NCIMB accession number 43405. The plant is resistant to ToLCNDV.

In one embodiment, the plant of the invention is a C. melo plant, which may comprise in its genome the modified PriL gene of the invention, wherein the modified PriL gene encodes a modified protein which may comprise one or more modifications in the wild type protein sequence of SEQ ID NO: 7. This plant is also referred to herein as a “C. melo plant of the invention”. When the C. melo plant of the invention may comprise the modified PriL gene of the invention homozygously, the plant is resistant to one or more of the following Begomoviruses selected from ToLCNDV and/or ToLCPMV and/or CuLCV and/or MCLCV and/or MLCV and/or SqLCV and/or CuLCrV and/or TYLCV and/or WmCSV and/or WmCMoV as a result of the presence of the modified protein.

In one embodiment, the C. melo plant of the invention may comprise in its genome a modified PriL gene, wherein the modified PriL gene encodes a modified protein which may comprise a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9. The C. melo plant of the invention is resistant to ToLCNDV.

In another embodiment, the modified PriL gene of the invention encodes a modified protein that may comprise a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9 and the modified PriL gene is homozygously present in a plant grown from a seed deposited under NCIMB accession number 43372. The plant is resistant to ToLCNDV.

The phrase “present in” may also mean “found in” or “contained in” or “obtainable from” (the genome of) plants grown from seeds of the deposit or the deposited seeds themselves. The phrases are intended to indicate that the modified PriL gene of the invention is the same or essentially the same as the modified PriL gene in the genome of the deposited material. “Essentially the same” means that the sequence of the modified PriL gene need not be identical in sequence but has in any case to perform the same function in causing the Begomovirus resistance as defined herein. In other words, the modified PriL gene may comprise polymorphisms (i.e. variation in the sequence) as compared to the modified PriL gene of the invention but these polymorphisms do not have any bearing on the function of the modified PriL gene in causing the resistance phenotype.

The invention also encompasses a seed which may comprise the modified PriL gene of the invention. The seed as described is also referred herein as “the seed of the invention”. When a seed of the invention may comprise a modified PriL gene homozygously, the encoded modified protein which may comprise one or more modifications(s) provides the plant grown from the seed with resistance to one or more of the following Begomoviruses selected from ToLCNDV and/or ToLCPMV and/or CuLCV and/or MCLCV and/or MLCV and/or SqLCV and/or CuLCrV and/or TYLCV and/or WmCSV and/or WmCMoV. The seed of the invention is a seed belonging to any one of the species C. moschata, C. pepo, C. melo, C. sativus, C. maxima, C. lanatus, S. lycopersicum, S. melongena or C. annuum, in particular a C. moschata, C. pepo or C. melo seed.

A plant grown from a seed of the invention also may comprise the modified PriL gene of the invention and is thus a plant of the invention. The invention also relates to seeds produced by a plant of the invention. These seeds comprise a modified PriL gene, and as such, a plant grown from said seed is a plant of the invention. When the seed may comprise the modified PriL gene of the invention homozygously the plant grown from this seed is resistant to one or more of the following Begomoviruses selected from ToLCNDV and/or ToLCPMV and/or CuLCV and/or MCLCV and/or MLCV and/or SqLCV and/or CuLCrV and/or TYLCV and/or WmCSV and/or WmCMoV as a result of the presence of the modified protein.

In one embodiment, the seed of the invention is a C. moschata or C. pepo seed which may comprise in its genome a modified PriL gene of the invention, wherein the modified PriL gene encodes a modified protein which may comprise one or more modifications in the wild type protein sequence of SEQ ID NO: 1, SEQ ID NO: 3, or SEQ ID NO: 11. This seed is also referred to herein as a “C. moschata or C. pepo seed of the invention”. When the C. moschata or C. pepo seed of the invention may comprise the modified PriL gene of the invention homozygously, the plant that is grown from the seed is resistant to one or more of the following Begomoviruses selected from ToLCNDV and/or ToLCPMV and/or CuLCV and/or MCLCV and/or MLCV and/or SqLCV and/or CuLCrV and/or TYLCV and/or WmCSV and/or WmCMoV as a result of the presence of the modified protein.

Preferably, the C. moschata or C. pepo seed of the invention may comprise in its genome a modified PriL gene, wherein the modified PriL gene encodes a modified protein which may comprise a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5. The C. moschata or C. pepo plant grown from the seed of the invention is resistant to ToLCNDV.

In one embodiment, the seed of the invention is a C. melo seed which may comprise in its genome a modified PriL gene of the invention, wherein the modified PriL gene encodes a modified protein which may comprise one or more modifications in the wild type protein sequence of SEQ ID NO: 7. This seed is also referred to herein as a “C. melo seed of the invention”. When the C. melo seed of the invention may comprise the modified PriL gene of the invention homozygously, the plant that is grown from the seed is resistant to one or more of the following Begomoviruses selected from ToLCNDV and/or ToLCPMV and/or CuLCV and/or MCLCV and/or MLCV and/or SqLCV and/or CuLCrV and/or TYLCV and/or WmCSV and/or WmCMoV as a result of the presence of the modified protein.

Preferably, the C. melo seed of the invention may comprise in its genome a modified PriL gene, wherein the modified PriL gene encodes a modified protein which may comprise a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9. The C. melo plant grown from the seed of the invention is resistant to ToLCNDV.

The invention further relates to progeny of the plants, cells, tissues and seeds of the invention, which progeny may comprise a modified PriL gene that leads to resistance to one or more of the following Begomoviruses selected from ToLCNDV and/or ToLCPMV and/or CuLCV and/or MCLCV and/or MLCV and/or SqLCV and/or CuLCrV and/or TYLCV and/or WmCSV and/or WmCMoV. Such progeny can in itself be plants, cells, tissues or seeds.

“Progeny” or “progeny plant of the invention” also encompass plants that comprise a modified PriL gene of the invention as herein described, and are obtained from other plants, or progeny of plants by vegetative propagation or multiplication or are plants grown from the seed of the invention. A progeny plant of the invention can comprise the modified PriL gene of the invention heterozygously, in which case the progeny plant is not resistant to Begomoviruses but is useful for transferring the modified PriL gene of the invention to further progeny plants. Progeny of the invention homozygously which may comprise a modified PriL gene in their genomes exhibit resistance to one or more of the following Begomoviruses selected from the group consisting of ToLCNDV, ToLCPMV, CuLCV, MCLCV, MLCV, SqLCV, CuLCrV, TYLCV, WmCSV and WmCMoV. As used herein “progeny” is intended to mean the first and all further descendants from a cross with a plant of the invention.

When a progeny plant of the invention may comprise a modified PriL gene homozygously, the encoded modified protein which may comprise one or more modification(s) imparts the progeny plant with resistance to one or more of the following Begomoviruses selected from the group consisting of ToLCNDV, ToLCPMV, CuLCV, MCLCV, MLCV, SqLCV, CuLCrV, TYLCV, WmCSV and WmCMoV. The progeny plant is a plant selected from any one of the species C. moschata, C. pepo, C. melo, C. sativus, C. maxima, C. lanatus, S. lycopersicum, S. melongena or C. annuum, in particular a C. moschata, C. pepo or C. melo progeny plant.

In one embodiment, the progeny plant is a progeny plant of the C. moschata or C. pepo plant of the invention which may comprise in its genome a modified PriL gene of the invention, wherein the modified PriL gene encodes a modified protein which may comprise one or more modifications in the wild type protein sequence of SEQ ID NO: 1, SEQ ID NO: 3, or SEQ ID NO: 11. This progeny plant is also referred to herein as a “C. moschata or C. pepo progeny plant of the invention”. When the C. moschata or C. pepo progeny plant of the invention may comprise the modified PriL gene of the invention homozygously, the progeny plant is resistant to ToLCNDV as a result of the presence of the modified protein.

Preferably, the C. moschata or C. pepo progeny plant of the invention may comprise in its genome a modified PriL gene, wherein the modified PriL gene encodes a modified protein which may comprise a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5. The C. moschata or C. pepo progeny plant of the invention is resistant to ToLCNDV.

In one embodiment, the progeny plant is a progeny plant of the C. melo plant of the invention which may comprise in its genome a modified PriL gene of the invention, wherein the modified PriL gene encodes a modified protein which may comprise one or more modifications in the wild type protein sequence of SEQ ID NO: 7. This progeny plant is referred to herein as a “C. melo progeny plant of the invention”. When the C. melo progeny plant of the invention may comprise the modified PriL gene of the invention homozygously, the progeny plant is resistant to ToLCNDV as a result of the presence of the modified protein.

Preferably, the C. melo progeny plant of the invention may comprise in its genome a modified PriL gene, wherein the modified PriL gene encodes a modified protein which may comprise a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9. The C. melo progeny plant of the invention is resistant to ToLCNDV.

The invention also relates to a fruit harvested from a plant of the invention or from a plant grown from a seed of the invention. This fruit is referred herein as a “fruit of the invention” and may comprise the modified PriL gene of the invention. A fruit of the invention is a fruit harvested from a plant of the invention or a plant grown from a seed of the invention.

When a fruit of the invention may comprise a modified PriL gene homozygously, the encoded modified protein which may comprise one or more modifications(s) provides the fruit with resistance to one or more of the following Begomoviruses selected from the group consisting of ToLCNDV, ToLCPMV, CuLCV, MCLCV, MLCV, SqLCV, CuLCrV, TYLCV, WmCSV and WmCMoV and thus the fruit has normal fruit characteristics that are agronomically acceptable. A fruit of the invention is a fruit belonging to any one of the species selected from C. moschata, C. pepo, C. melo, C. sativus, C. maxima, C. lanatus, S. lycopersicum, S. melongena or C. annuum, in particular a C. moschata, C. pepo or C. melo fruit.

In one embodiment, the fruit is a C. moschata or C. pepo fruit which may comprise in its genome a modified PriL gene of the invention, wherein the modified PriL gene encodes a modified protein which may comprise one or more modifications in the wild type protein sequence of SEQ ID NO: 1, SEQ ID NO: 3, or SEQ ID NO: 11. This fruit is also referred to herein as a “C. moschata or C. pepo fruit of the invention”. When the C. moschata or C. pepo fruit of the invention may comprise the modified PriL gene of the invention homozygously, the fruit is resistant to one or more of the following Begomoviruses selected from the group consisting of ToLCNDV, and/or ToLCPMV, and/or CuLCV, and/or MCLCV, and/or MLCV, and/or SqLCV, and/or CuLCrV, and/or TYLCV, and/or WmCSV and/or WmCMoV as a result of the presence of the modified protein and thus has normal fruit characteristics that are agronomically acceptable. The seeds of this fruit also comprise the modified PriL gene of the invention and therefore also form a part of the invention.

Preferably, the C. moschata or C. pepo fruit of the invention may comprise in its genome a modified PriL gene, wherein the modified PriL gene encodes a modified protein which may comprise a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5. The C. moschata or C. pepo fruit of the invention is resistant to ToLCNDV and has normal fruit characteristics that are agronomically acceptable.

In one embodiment, the fruit is a C. melo fruit which may comprise in its genome a modified PriL gene of the invention, wherein the modified PriL gene encodes a modified PriL protein which may comprise one or more modifications in the wild type protein sequence of SEQ ID NO: 7. This fruit is also referred to herein as a “C. melo fruit of the invention”. When the C. melo fruit of the invention may comprise the modified PriL gene of the invention homozygously, the fruit is resistant to one or more of the Begomoviruses selected from the group consisting of ToLCNDV, ToLCPMV, CuLCV, MCLCV, MLCV, SqLCV, CuLCrV, TYLCV, WmCSV and WmCMoV as a result of the presence of the modified protein and has normal fruit characteristics that are agronomically acceptable. The seeds of the fruit also comprise the modified PriL gene of the invention and therefore also form a part of the invention.

Preferably, the C. melo fruit of the invention may comprise in its genome a modified PriL gene, wherein the modified PriL gene encodes a modified protein which may comprise a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9. The C. melo fruit of the invention is resistant to ToLCNDV and has normal fruit characteristics that are agronomically acceptable.

Moreover the invention also relates to a food product or a processed food product which may comprise the fruit of the invention or part thereof. The food product may have undergone one or more processing steps. Such a processing step might comprise but is not limited to any one of the following treatments or combinations thereof: peeling, cutting, washing, juicing, cooking, cooling or preparing a salad mixture which may comprise the fruit of the invention. The processed form that is obtained is also part of this invention.

The invention further relates to a cell of a plant of the invention. Such a cell may either be in isolated form or a part of the complete plant or parts thereof and still constitutes a cell of the invention because such a cell harbors the genetic information that imparts resistance to one or more of the Begomoviruses selected from the group consisting of ToLCNDV, ToLCPMV, CuLCV, MCLCV, MLCV, SqLCV, CuLCrV, TYLCV, WmCSV and WmCMoV a plant of the invention. Each cell of a plant of the invention carries the genetic information that leads to ToLCNDV resistance. A cell of the invention may also be a regenerable cell that can regenerate into a new plant of the invention. The presence of genetic information as used herein is the presence of a modified PriL gene as defined herein.

When a cell of the invention may comprise a modified PriL gene homozygously, the encoded modified protein which may comprise one or more modification(s) provides the cell with the genetic information that leads to resistance to one or more of the Begomoviruses selected from the group consisting of ToLCNDV, ToLCPMV, CuLCV, MCLCV, MLCV, SqLCV, CuLCrV, TYLCV, WmCSV and WmCMoV. The cell of the invention is a cell belonging to any one of the species selected from C. moschata, C. pepo, C. melo, C. sativus, C. maxima, C. lanatus, S. lycopersicum, S. melongena or C. annuum, in particular a C. moschata, C. pepo or C. melo cell.

In one embodiment, the cell is a C. moschata or C. pepo cell which may comprise in its genome a modified PriL gene, wherein the modified PriL gene encodes a modified protein which may comprise one or more modifications in the wild type protein sequence of SEQ ID NO: 1, SEQ ID NO: 3, or SEQ ID NO: 11. This cell is also referred to herein as a “C. moschata or C. pepo cell of the invention”. When the C. moschata or C. pepo cell of the invention may comprise the modified PriL gene homozygously, the cell provides genetic information that leads to resistance to one or more of the Begomoviruses selected from the group consisting of ToLCNDV, ToLCPMV, CuLCV, MCLCV, MLCV, SqLCV, CuLCrV, TYLCV, WmCSV and WmCMoV.

Preferably, the C. moschata or C. pepo cell of the invention may comprise in its genome a modified PriL gene, wherein the modified PriL gene encodes a modified protein which may comprise a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5. The C. moschata or C. pepo cell of the invention provides genetic information that leads to ToLCNDV resistance.

In one embodiment, the cell is a C. melo cell which may comprise in its genome a modified PriL gene of the invention, wherein the modified PriL gene encodes a modified protein which may comprise one or more modifications in the wild type protein sequence of SEQ ID NO: 7. This cell is also referred to herein as a “C. melo cell of the invention”. When the C. melo cell of the invention may comprise the modified PriL gene homozygously, the cell provides genetic information that leads to resistance to one or more of the Begomoviruses selected from the group consisting of ToLCNDV, ToLCPMV, CuLCV, MCLCV, MLCV, SqLCV, CuLCrV, TYLCV, WmCSV and WmCMoV.

Preferably, the C. melo cell of the invention may comprise in its genome a modified PriL gene, wherein the modified PriL gene encodes a modified protein which may comprise a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9. The C. melo cell of the invention provides genetic information that leads to ToLCNDV resistance.

The invention further relates to a tissue culture of a plant of the invention, wherein the tissue culture may comprise the modified PriL gene of the invention that leads to the ToLCNDV resistance of the invention. Such tissue culture can be selected or derived from any part of the plant, in particular from leaves, pollen, embryos, cotyledon, hypocotyls, meristematic cells, roots, root tips, anthers, flowers, seeds, and stems. The tissue culture can be regenerated into a plant which may comprise the modified PriL gene, wherein the regenerated plant expresses the Begomovirus resistance of the invention. The regenerated plant is also part of the invention.

When a tissue culture of a plant of the invention may comprise a modified PriL gene homozygously, the modified protein which may comprise one or more modification(s) provides the tissue culture with the genetic information that leads to resistance to one or more of the Begomoviruses selected from the group consisting of ToLCNDV, ToLCPMV, CuLCV, MCLCV, MLCV, SqLCV, CuLCrV, TYLCV, WmCSV and WmCMoV. The tissue culture is a tissue culture of a plant of any one of the species selected from C. moschata, C. pepo, C. melo, C. sativus, C. maxima, C. lanatus, S. lycopersicum, S. melongena or C. annuum, in particular a C. moschata, C. pepo or C. melo tissue culture.

In one embodiment, the invention relates to a tissue culture of a C. moschata or C. pepo plant of the invention, wherein the tissue culture may comprise a modified PriL gene, wherein the modified PriL gene encodes a modified protein which may comprise one or more modifications in the wild type protein sequence of SEQ ID NO: 1, SEQ ID NO: 3, or SEQ ID NO: 11. This tissue culture is also referred to herein as a “C. moschata or C. pepo tissue culture of the invention”. When the C. moschata or C. pepo tissue culture of the invention may comprise the modified PriL gene homozygously, the tissue culture provides genetic information that leads to resistance to one or more of the Begomoviruses selected from the group consisting of ToLCNDV, ToLCPMV, CuLCV, MCLCV, MLCV, SqLCV, CuLCrV, TYLCV, WmCSV and WmCMoV.

Preferably, the C. moschata or C. pepo tissue culture of the invention may comprise a modified PriL gene, wherein the modified PriL gene encodes a modified protein which may comprise a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5. The C. moschata or C. pepo tissue culture of the invention provides genetic information that leads to ToLCNDV resistance.

In one embodiment, the invention relates to a tissue culture of a C. melo plant of the invention, wherein the tissue culture may comprise a modified PriL gene, wherein the modified PriL gene encodes a modified protein which may comprise one or more modifications in the wild type protein sequence of SEQ ID NO: 7. This tissue culture is also referred to herein as a “C. melo tissue culture of the invention”. When the C. melo tissue culture of the invention may comprise the modified PriL gene homozygously, the tissue culture provides genetic information that leads to resistance to one or more of the Begomoviruses selected from the group consisting of ToLCNDV, ToLCPMV, CuLCV, MCLCV, MLCV, SqLCV, CuLCrV, TYLCV, WmCSV and WmCMoV.

Preferably, the C. melo tissue culture of the invention may comprise a modified PriL gene, wherein the modified PriL gene encodes a modified protein which may comprise a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9. The C. melo tissue culture of the invention provides genetic information that leads to ToLCNDV resistance.

The invention also relates to propagation material suitable for producing a plant of the invention, wherein the propagation material is suitable for sexual reproduction, and is in particular selected from a microspore, pollen, ovary, ovule, embryo sac and egg cell, or is suitable for vegetative reproduction, and is in particular selected from a cutting, root, stem cell, and protoplast, or is suitable for tissue culture of regenerable cells or protoplasts, which regenerable cells or protoplasts are in particular selected from a leaf, pollen, embryo, cotyledon, hypocotyl, meristematic cell, root, root tip, anther, flower and stem.

When the propagation material and the plant produced from the propagation material may comprise a modified PriL gene homozygously, the modified protein which may comprise one or more modification(s) imparts the material with the genetic information that leads to resistance to one or more of the Begomoviruses selected from the group consisting of ToLCNDV, ToLCPMV, CuLCV, MCLCV, MLCV, SqLCV, CuLCrV, TYLCV, WmCSV and WmCMoV.

This propagation material is referred herein as “propagation material of the invention”. A plant of the invention may be used as a source of the propagation material. The propagation material is derived from or is suitable for producing a plant of any one of the species selected from C. moschata, C. pepo, C. melo, C. sativus, C. maxima, C. lanatus, S. lycopersicum, S. melongena or C. annuum, in particular a C. moschata, C. pepo or C. melo propagation material.

In one embodiment, the invention relates to propagation material suitable for producing a C. moschata or C. pepo plant of the invention, wherein the propagation material may comprise a modified PriL gene, wherein the modified PriL gene encodes a modified protein which may comprise one or more modifications in the wild type protein sequence of SEQ ID NO: 1, SEQ ID NO: 3, or SEQ ID NO: 11. This propagation material is also referred to herein as a “C. moschata or C. pepo propagation material of the invention”. When the C. moschata or C. pepo propagation material of the invention may comprise the modified PriL gene homozygously, the propagation material provides the plant that is produced with genetic information that imparts resistance to one or more of the Begomoviruses selected from the group consisting of ToLCNDV, ToLCPMV, CuLCV, MCLCV, MLCV, SqLCV, CuLCrV, TYLCV, WmCSV and WmCMoV.

Preferably, the C. moschata or C. pepo propagation material of the invention may comprise a modified PriL gene, wherein the modified PriL gene encodes a modified protein which may comprise a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5. The C. moschata or C. pepo propagation material of the invention provides the plant that is produced with genetic information that leads to ToLCNDV resistance.

In one embodiment, the invention relates to propagation material suitable for producing a C. melo plant of the invention, wherein the propagation material may comprise a modified PriL gene, wherein the modified PriL gene encodes a modified protein which may comprise one or more modifications in the wild type protein sequence of SEQ ID NO: 7. This propagation material is also referred to herein as “C. melo propagation material of the invention”. When the C. melo propagation material of the invention may comprise the modified PriL gene homozygously, the propagation material provides the plant that is produced with genetic information that imparts resistance to one or more of the Begomoviruses selected from the group consisting of ToLCNDV, ToLCPMV, CuLCV, MCLCV, MLCV, SqLCV, CuLCrV, TYLCV, WmCSV and WmCMoV.

Preferably, the C. melo propagation material of the invention may comprise a modified PriL gene, wherein the modified PriL gene encodes a modified protein which may comprise a a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9. The C. melo propagation material of the invention provides the plant that is produced with genetic information that leads to ToLCNDV resistance.

The invention further relates to plant tissue of a plant of the invention. The tissue can be undifferentiated tissue or already differentiated tissue. Undifferentiated tissues are for example stem tips, anthers, petals, pollen and can be used in micropropagation to obtain new plantlets that are grown into plants of the invention. The tissue can also be grown from a cell of the invention.

The invention further relates to parts of a plant of the invention that are suitable for sexual reproduction. Such parts are for example selected from the group consisting of microspores, pollen, ovaries, ovules, embryo sacs, and egg cells. Additionally the invention relates to parts of a plant of the invention that are suitable for vegetative reproduction, which are in particular cuttings, roots, stems, cells, protoplasts. The parts of the plants as previously mentioned are considered propagation material. The plant that is produced from the propagation material may comprise a modified PriL gene that leads to resistance to one or more of the Begomoviruses selected from the group consisting of ToLCNDV, ToLCPMV, CuLCV, MCLCV, MLCV, SqLCV, CuLCrV, TYLCV, WmCSV and WmCMoV.

The invention further relates to the germplasm of plants of the invention. The germplasm is constituted by all inherited characteristics of an organism and according to the invention encompasses at least the trait of the invention. The germplasm can be used in a breeding program for the development of plants resistant to one or more of the Begomoviruses selected from the group consisting of ToLCNDV, ToLCPMV, CuLCV, MCLCV, MLCV, SqLCV, CuLCrV, TYLCV, WmCSV and WmCMoV. The use of germplasm that may comprise a modified PriL gene leading to Begomovirus resistance in breeding is also part of the present invention.

The invention also relates to the use of a modified PriL gene for producing a plant that is resistant to one or more of the Begomoviruses selected from the group consisting of ToLCNDV, ToLCPMV, CuLCV, MCLCV, MLCV, SqLCV, CuLCrV, TYLCV, WmCSV and WmCMoV, preferably a plant of the species C. moschata, C. pepo, C. melo, C. sativus, C. maxima, C. lanatus, S. lycopersicum, S. melongena or C. annuum, in particular a C. moschata, C. pepo or C. melo plant is suitably produced. The Begomovirus resistant plant may be produced by introducing the modified PriL gene of the invention into its genome, by means of mutagenesis or introgression, or combinations thereof.

The invention additionally further relates to the use of a plant of the invention in plant breeding. The invention thus relates to a breeding method for the development of plants that are resistant to one or more of the Begomoviruses selected from the group consisting of ToLCNDV, ToLCPMV, CuLCV, MCLCV, MLCV, SqLCV, CuLCrV, TYLCV, WmCSV and WmCMoV, wherein the germplasm which may comprise said resistance is used. Seed being representative for the germplasm was deposited with NCIMB under accession number NCIMB 43405 or NCIMB 43372.

The invention further relates to plants of the invention that have acquired a modified PriL gene from a suitable source, either by conventional breeding, or genetic modification, in particular by cisgenesis or transgenesis. Cisgenesis is a genetic modification of plants with a natural gene, encoding a (agricultural trait) from the crop plant itself or from a sexually compatible donor plant. Transgenesis is a genetic modification of a plant with a gene from a non-crossable species or with a synthetic gene.

The source from which a modified PriL gene can be acquired, is formed by plants grown from seeds of which a representative sample was deposited under accession number NCIMB 43405 or NCIMB 43372, or from the deposited seeds NCIMB 43405 or NCIMB 43372 themselves, or from sexual or vegetative descendants thereof, or from another source which may comprise the modified PriL gene as defined herein that leads to resistance to one or more of the Begomoviruses selected from the group consisting of ToLCNDV, ToLCPMV, CuLCV, MCLCV, MLCV, SqLCV, CuLCrV, TYLCV, WmCSV and WmCMoV, or from any combination of these sources.

To obtain a modified PriL gene from a source in which it is heterozygously present, a seed of such a plant may be grown and flowers pollinated from the same plant or from another plant that also has a heterozygous modified PriL gene to obtain a fruit with seeds. When these seeds are sown, the resulting plants will segregate according to normal segregation ratios, which means about 25% of the plants will have the modified PriL gene homozygously present, about 50% of the plants will have the modified PriL gene heterozygously present and about 25% of the plants will not have the modified PriL gene. For the selection of a preferred plant having a modified PRiL gene either homozygously or heterozygously, the presence of a modified PRiL gene can be determined by sequencing the PriL gene or by markers developed based on the sequences that are described herein. Alternatively, plants can be phenotypically observed and visually selected for the presence of resistance to one or more of the Begomoviruses selected from the group consisting of ToLCNDV, ToLCPMV, CuLCV, MCLCV, MLCV, SqLCV, CuLCrV, TYLCV, WmCSV and WmCMoV. The skilled person is aware of how to work with genes in heterozygous and homozygous form using known breeding and selection procedures.

The invention also relates to a method for producing a Begomovirus resistant plant, said method which may comprise:
(a) crossing a plant which may comprise a modified PriL gene with another plant to obtain an F1 population;
(b) optionally performing one or more rounds of selfing and/or crossing a plant from the F1 to obtain a further generation population;
(c) selecting from the population a plant that may comprise the modified PriL gene and is resistant against one or more of the Begomoviruses selected from the group consisting of ToLCNDV, ToLCPMV, CuLCV, MCLCV, MLCV, SqLCV, CuLCrV, TYLCV, WmCSV and WmCMoV.

The invention also relates to a method for producing a Begomovirus resistant plant, said method which may comprise:
(a) introducing one or more mutations in a population of plants;
(b) selecting a plant showing resistance to one or more Begomovirus selected from the group consisting of ToLCNDV, ToLCPMV, CuLCV, MCLCV, MLCV, SqLCV, CuLCrV, TYLCV, WmCSV and WmCMoV;
(c) verifying if the plant selected in step (b) has a mutation in its PriL gene, and selecting a plant which may comprise such a mutation;
(d) growing the plant obtained in step (c);
   wherein the wild type PriL gene encodes a protein which may comprise at least 60% sequence identity, preferably at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1 or SEQ ID NO: 3.

In one embodiment, the modified PriL gene in the above method for the production of a Begomovirus resistant plant, may comprise a SNP that results in a phenylalanine (F) to cysteine (C) substitution at a position corresponding to position 147 according to SEQ ID NO: 5, and/or a glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5, and/or wherein the one or more modifications in the PriL gene is a SNP that results in a tyrosine (Y) to histidine (H) substitution at a position corresponding to position 4 according to SEQ ID NO: 9, and/or proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9.

In one embodiment the invention relates to a method for the production of a C. moschata plant that is resistant to one or more Begomoviruses selected from the group consisting of ToLCNDV, ToLCPMV, CuLCV, MCLCV, MLCV, SqLCV, CuLCrV, WmCSV and WmCMoV, said method which may comprise:
(a) crossing a C. moschata plant of the invention which may comprise a modified PriL gene of the invention with another C. moschata parent plant not which may comprise the modified PriL gene, to obtain an F1 population;
(b) optionally performing selfing an F1 plant to obtain an F2 population;
(c) backcrossing an F1 or an F2 plant with the preferred parent to obtain a BC1 population; and,
(d) optionally selfing a BC1 plant to obtain a BC1F2 population;
(e) selecting in the BC1 or BC1F2 population for a C. moschata plant that may comprise the modified PriL gene and is resistant to one or more Begomoviruses selected from the group consisting of ToLCNDV, ToLCPMV, CuLCV, MCLCV, MLCV, SqLCV, CuLCrV, WmCSV and WmCMoV, suitably by sequencing the PriL gene. The plant can also be phenotypically selected for having resistance to one or more Begomoviruses selected from the group consisting of ToLCNDV, ToLCPMV, CuLCV, MCLCV, MLCV, SqLCV, CuLCrV, WmCSV and WmCMoV. The backcrossing, selfing and selection steps may optionally be repeated one to ten more times to produce further backcross progeny which may comprise the modified PriL gene and which is resistant to one or more Begomoviruses selected from the group consisting of ToLCNDV, ToLCPMV, CuLCV, MCLCV, MLCV, SqLCV, CuLCrV, WmCSV and WmCMoV.

In one embodiment the invention relates to a method for the production of a C. pepo plant that is resistant to one or more Begomoviruses selected from the group consisting of ToLCNDV, ToLCPMV, CuLCV, MCLCV, MLCV, SqLCV, CuLCrV, TYLCV, WmCSV and WmCMoV, said method which may comprise:
(a) crossing a C. moschata or C. pepo plant of the invention which may comprise a
   modified PriL gene of the invention with another C. pepo parent plant not which may comprise the modified PriL gene, to obtain an F1 population;
(b) optionally performing selfing an F1 plant to obtain an F2 population;
(c) backcrossing an F1 or an F2 plant with the preferred parent to obtain a BC1 population; and,
(d) optionally selfing a BC1 plant to obtain a BC1F2 population;
(e) selecting in the BC1 or BC1F2 population for a C. pepo plant that may comprise the modified PriL gene and is resistant to one or more Begomoviruses selected from the group consisting of ToLCNDV, ToLCPMV, CuLCV, MCLCV, MLCV, SqLCV, CuLCrV, WmCSV and WmCMoV, suitably by sequencing the PriL gene. The plant can also be phenotypically selected for having resistance to one or more Begomoviruses selected from the group consisting of ToLCNDV, ToLCPMV, CuLCV, MCLCV, MLCV, SqLCV, CuLCrV, WmCSV and WmCMoV. The backcrossing, selfing and selection steps may optionally be repeated one to ten more times to produce further backcross progeny which may comprise the modified PriL gene and which is resistant to one or more Begomoviruses selected from the group consisting of ToLCNDV, ToLCPMV, CuLCV, MCLCV, MLCV, SqLCV, CuLCrV, WmCSV and WmCMoV.

In one embodiment the invention relates to a method for the production of a C. melo plant that is resistant to one or more Begomoviruses selected from the group consisting of ToLCNDV, ToLCPMV, CuLCV, MCLCV, MLCV, SqLCV, CuLCrV, WmCSV and WmCMoV, said method which may comprise:
(a) crossing a C. melo plant of the invention which may comprise a modified PriL gene of the invention with another C. melo parent plant not which may comprise the modified PriL gene, to obtain an F1 population;
(b) optionally performing selfing an F1 plant to obtain an F2 population;
(c) backcrossing an F1 or an F2 plant with the preferred parent to obtain a BC1 population; and,
(d) optionally selfing a BC1 plant to obtain a BC1F2 population;
(e) selecting in the BC1 or BC1F2 population for a C. melo plant that may comprise the modified PriL gene and is resistant to one or more Begomoviruses selected from the group consisting of ToLCNDV, ToLCPMV, CuLCV, MCLCV, MLCV, SqLCV, CuLCrV, WmCSV and WmCMoV, suitably by sequencing the PriL gene. The plant can also be phenotypically selected for having resistance to one or more Begomoviruses selected from the group consisting of ToLCNDV, ToLCPMV, CuLCV, MCLCV, MLCV, SqLCV, CuLCrV, WmCSV and WmCMoV. The backcrossing, selfing and selection steps may optionally be repeated one to ten more times to produce further backcross progeny which may comprise the modified PriL gene and which is resistant to one or more Begomoviruses selected from the group consisting of ToLCNDV, ToLCPMV, CuLCV, MCLCV, MLCV, SqLCV, CuLCrV, WmCSV and WmCMoV.

The invention further relates to a method of introducing another desired trait into a Begomovirus resistant plant, said method which may comprise:
(a) crossing a plant of the invention which may comprise a modified PriL gene with a
   second plant that may comprise the other desired trait to produce F1 progeny;
(b) selecting an F1 progeny that may comprise the modified PriL gene and the desired trait;
(c) crossing the selected F1 progeny which may comprise the modified PriL gene with
   either parent, to produce backcross progeny;
(d) selecting backcross progeny which may comprise the modified PriL gene and the
   other desired trait; and,
(e) optionally repeating steps c) and d) one or more times in succession to produce selected fourth or higher backcross progeny that may comprise the desired trait and is resistant to one or more Begomoviruses selected from the group consisting of ToLCNDV, ToLCPMV, CuLCV, MCLCV, MLCV, SqLCV, CuLCrV, TYLCV, WmCSV and WmCMoV.

The plant of the invention used in the any of the methods for the production of a Begomovirus resistant plant, can be any plant or progeny plant of the invention as described herein, or can also be a plant grown from seed deposited under NCIMB accession number 43405 or 43372.

The invention further relates to a method for the production of a Begomovirus resistant plant which may comprise a modified PriL gene that leads to resistance to one or more Begomoviruses selected from the group consisting of ToLCNDV, ToLCPMV, CuLCV, MCLCV, MLCV, SqLCV, CuLCrV, TYLCV, WmCSV and WmCMoV, by using tissue culture of plant material that may comprise a modified PriL gene in its genome.

The invention further relates to a method for the production of a Begomovirus resistant plant which may comprise a modified PriL gene that leads to resistance to one or more Begomoviruses selected from the group consisting of ToLCNDV, ToLCPMV, CuLCV, MCLCV, MLCV, SqLCV, CuLCrV, TYLCV, WmCSV and WmCMoV, by using vegetative reproduction of plant material that may comprise a modified PriL gene in its genome.

The invention further provides a method for the production of a Begomovirus resistant plant by using doubled haploid generation techniques to generate a doubled haploid line that homozygously may comprise a modified PriL gene and is resistant against one or more Begomoviruses selected from the group consisting of ToLCNDV, ToLCPMV, CuLCV, MCLCV, MLCV, SqLCV, CuLCrV, TYLCV, WmCSV and WmCMoV.

The invention further relates to a method for the production of a Begomovirus resistant plant which may comprise a modified PriL gene that imparts the plant with resistance to one or more Begomoviruses selected from the group consisting of ToLCNDV, ToLCPMV, CuLCV, MCLCV, MLCV, SqLCV, CuLCrV, TYLCV, WmCSV and WmCMoV, which method may comprise growing a seed which may comprise a modified PriL gene into said plant. In one embodiment, the seed used in the method may be seed deposited under NCIMB accession number 43405, or progeny seed thereof. In another embodiment, the seed used in the method may be seed deposited under NCIMB accession number 43372, or progeny seed thereof

The invention further relates to a method for seed production which may comprise growing a Begomovirus resistant plant from seeds of the invention, allowing the plants to produce fruits with seeds, and harvesting those seeds. Production of the seeds is suitably done by crossing or selfing. Preferably the seeds that are so produced have the capability to grow into plants that are resistant to one or more Begomoviruses selected from the group consisting of ToLCNDV, ToLCPMV, CuLCV, MCLCV, MLCV, SqLCV, CuLCrV, TYLCV, WmCSV and WmCMoV.

The invention further relates to hybrid seed and to a method for producing said hybrid seed, which may comprise crossing a first parent plant with a second parent plant and harvesting the resultant hybrid seed, wherein the first parent plant and/or the second parent plant is a plant of the invention. The resultant hybrid plant which may comprise a modified PriL gene of the invention and exhibiting resistance to one or more Begomoviruses selected from the group consisting of ToLCNDV, ToLCPMV, CuLCV, MCLCV, MLCV, SqLCV, CuLCrV, TYLCV, WmCSV and WmCMoV, is also a plant of the invention.

It is clear that the parent that provides the trait of the invention is not necessarily a plant grown directly from the deposited seeds. The parent can also be a progeny plant from the seed or a progeny plant from sees that are identified to have the trait of the invention by other means.

Introgression of a modified PriL gene as used herein means introduction of the modified PriL gene from a donor plant which may comprise said modified PriL gene into a recipient plant not carrying said modified PriL gene by standard breeding techniques wherein selection for plants which may comprise the modified PriL gene can be performed phenotypically by means of observation of the resistance to ToLCNDV, or selection can be performed with the use of markers through marker assisted breeding, or combinations of these. Selection is started in the F1 or any further generation from a cross between the recipient plant and the donor plant, suitably by using markers developed based on the sequence of the modified PriL gene. The skilled person is familiar with creating and using molecular markers that can be used to identify or are linked to the trait of the invention. Development and use of such markers for identifying and selecting plants of the invention also form part of the invention.

The invention also relates to a method for identifying or selecting a Begomovirus resistant plant, said method which may comprise:
(a) assaying genomic nucleic acids of a plant for the presence of one or more modifications in the PriL gene;
(b) identifying or selecting a plant if one or more modifications in the PriL gene are present; and,
(c) optionally verifying if the plant is resistant to one or more Begomoviruses selected from the group consisting of ToLCNDV, ToLCPMV, CuLCV, MCLCV, MLCV, SqLCV, CuLCrV, TYLCV, WmCSV and WmCMoV.

In one embodiment, the one or more modifications in the PriL gene in the above method for identifying or selecting a Begomovirus resistant plant is a SNP that results in a phenylalanine (F) to cysteine (C) substitution at a position corresponding to position 147 according to SEQ ID NO: 5, and/or a glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5, and/or wherein the one or more modifications in the PriL gene is a SNP that results in a tyrosine (Y) to histidine (H) substitution at a position corresponding to position 4 according to SEQ ID NO: 9, and/or proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9.

The presence of the one or more modifications in the PriL gene can also suitably be identified for example by sequencing the modified PriL gene of any one of the species: Cucurbita moschata, Cucurbita pepo, Cucumis melo, Cucumis sativus, Cucurbita maxima, Citrullus lanatus, Solanum lycopersicum, Solanum melongena or Capsicum annuum described herein and comparing it with its respective wild type PriL gene sequence (SEQ ID NOS: 2, 4, 8, 12, 14, 16, 18, 20, 22, 24, 26, 28, or 30). The skilled person is familiar with techniques available in the art for determining the genomic DNA or the coding DNA sequence. These techniques are for example PCR amplification followed by Sanger sequencing or whole genome sequencing.

Additionally the modifications in the PriL gene can suitably be identified by using markers developed based on the sequence of the modified PriL gene. The skilled person is familiar with creating and using molecular markers that can be used to identify or are linked to the trait of the invention.

In one embodiment, the invention relates to a marker for identifying a modified PriL gene, wherein the marker detects a thymine (T) to a guanine (G) SNP at a position corresponding to position 440 according to the wild type PriL gene sequence of SEQ ID NO: 2, or a guanine at an adenine SNP corresponding to position 485 according to the wild type PriL gene sequence of SEQ ID NO: 2, or a guanine to a cytosine SNP at a position corresponding to position 489 according to the wild type PriL gene sequence of SEQ ID NO: 2, or a thymine to cytosine SNP at a position corresponding to position 10 according to the wild type PriL gene sequence of SEQ ID NO: 8, or a cytosine to an adenine SNP at a position corresponding to position 497 according to the wild type PriL gene sequence of SEQ ID NO: 8, or a thymine to a cytosine SNP at a position corresponding to position 599 according to the wild type PriL gene sequence of SEQ ID NO: 8. The use of said marker(s) for identifying or selecting a Begomovirus resistant plant which may comprise a modified PriL gene also forms part of the invention.

TABLE 1
Sequence Information

| | | SEQ | | |
|---|---|---|---|---|
| | | ID | URL (if | |
| Species | Detail | NO: | applicable) | Sequence |
| Cucurbita | PriL | 1 | https://www. | MELHRSQRKSSTATISSTTLPLYRSAPPLEVRL |
| moschata | isoform | | ncbi.nlm. | EEFELYAIDRLRVLKGISDGLSRGKKSEDMEK |
| cv. Rifu | 1 | | nih.gov/ | LVRDLLKAHMKHPQASEAVNKDIISHFVLRLV |
| | Protein | | protein/xp_ | YCRTEDLRKWFLSMETMLFRHRFLSESPESQK |
| | | | 022959574.1 | QVFSELGLSYKAIGYAEFEAVKDKLVQVARLI |
| | | | | GQPVPSADAIYYKVPWEEVPELVAGRRVLLH |
| | | | | KGYAYIAIYQVVSLVATQFRSYLSKALSLTNR |
| | | | | KWTSTIREQEKDRLTPIVEALCTSYLGPDYSQP |
| | | | | TEYADISIKDLEQIAKSSFPLCMRHLFDKLKED |
| | | | | HHLKHGGRMQLGLFLKGVGLKLDDALAFWR |
| | | | | AEFSQRVGVERFDKEYAYSIRHNYGKEGKRV |
| | | | | DYSPYSCQKVISSSPGVGDHHGCPYRHFSEDN |
| | | | | LRAALGKMGVNNQKMEDILDKVRNRHYQLA |
| | | | | CTLTFESVHGSSCDAGINHPNQYFIDSQKVLQS |
| | | | | KVDPETSSCTNFDAFLTVLSLGRLST |
| Cucurbita | PriL | 2 | https://www. | ATGGAACTCCATCGTTCTCAGAGGAAATCTT |
| moschata | isoform | | ncbi.nlm. | CCACCGCGACCATTTCCTCGACCACTCTTCC |
| cv. Rifu | 1 CDS | | nih.gov/ | ACTCTATCGCTCCGCTCCTCCTCTCGAAGTC |
| | | | nuccore/XM_ | AGGCTTGAAGAATTCGAACTTTATGCCATAG |
| | | | 023103806.1 | ATCGTCTTCGAGTTCTTAAAGGAATTTCTGA |
| | | | | TGGTTTATCTCGAGGAAAGAAATCTGAAGA |
| | | | | CATGGAGAAACTGGTTAGAGACTTGTTGAA |
| | | | | GGCCCATATGAAACATCCACAGGCATCCGA |
| | | | | GGCTGTGAACAAGGATATAATATCTCACTTT |
| | | | | GTTCTGCGCCTCGTATACTGCAGAACGGAGG |
| | | | | ACTTGAGAAAATGGTTTCTTTCTATGGAAAC |
| | | | | TATGCTATTTCGACACCGTTTTCTTTCTGAAA |
| | | | | GTCCTGAATCTCAGAAGCAGGTCTTTTCGGA |
| | | | | GCTTGGCCTCTCATACAAAGCAATCGGTTAT |
| | | | | GCAGAGTTTGAGGCTGTGAAGGACAAATTG |
| | | | | GTCCAAGTTGCTCGGTTGATTGGTCAGCCTG |
| | | | | TACCAAGCGCTGATGCAATATACTATAAGGT |
| | | | | ACCATGGGAAGAAGTTCCAGAACTGGTGGC |
| | | | | TGGTCGAAGGGTATTACTTCATAAAGGATAT |
| | | | | GCATATATTGCTATCTATCAGGTGGTTTCCC |
| | | | | TTGTTGCAACACAATTCCGCAGTTACCTATC |
| | | | | AAAGGCCTTAAGTCTGACAAACAGGAAATG |
| | | | | GACATCTACAATAAGAGAACAAGAGAAGGA |
| | | | | TCGGTTGACCCCTATCGTTGAAGCCCTTTGC |
| | | | | ACTAGCTACCTGGGTCCTGACTACTCACAGC |
| | | | | CAACAGAGTATGCTGATATATCAATCAAAG |
| | | | | ACCTTGAACAAATTGCTAAAAGTTCATTTCC |
| | | | | TCTTTGCATGCGGCACCTATTTGATAAGCTG |
| | | | | AAAGAAGATCATCATTTGAAGCATGGAGGG |
| | | | | AGAATGCAATTAGGTCTCTTTCTTAAGGGTG |
| | | | | TTGGTTTGAAGCTTGATGATGCCCTTGCTTTC |
| | | | | TGGAGAGCTGAGTTCTCGCAGAGAGTTGGT |
| | | | | GTTGAGAGGTTTGACAAAGAATATGCATAC |
| | | | | AGTATCAGGCATAACTATGGAAAAGAAGGC |
| | | | | AAGAGAGTGGATTATTCACCTTATTCCTGTC |
| | | | | AAAAAGTCATCTCATCATCACCTGGTGTTGG |
| | | | | AGATCATCATGGATGTCCCTATAGACATTTC |
| | | | | AGTGAAGACAACTTAAGAGCAGCTCTTGGT |
| | | | | AAAATGGGAGTAAATAACCAGAAAATGGAA |
| | | | | GATATATTGGACAAAGTGCGAAATAGACAC |
| | | | | TATCAGTTGGCCTGCACCTTGACATTTGAAT |
| | | | | CGGTTCATGGTTCGTCATGCGACGCCGGGAT |
| | | | | TAATCATCCAAACCAGTACTTCATTGATAGT |
| | | | | CAAAAAGTTTTGCAATCTAAGGTAGACCCTG |
| | | | | AAACTTCATCATGTACTAATTTTGATGCTTTC |
| | | | | TTGACAGTTTTGAGTCTTGGAAGGCTGAGCA |
| | | | | CATAA |
| Cucurbita | PriL | 3 | https://www. | MELHRSQRKSSTATISSTTLPLYRSAPPLEVRL |
| moschata | isoform | | ncbi.nlm. | EEFELYAIDRLRVLKGISDGLSRGKKSEDMEK |
| cv. Rifu | 2 | | nih.gov/ | LVRDLLKAHMKHPQASEAVNKDIISHFVLRLV |
| | Protein | | protein/XP_ | YCRTEDLRKWFLSMETMLFRHRFLSESPESQK |
| | | | 022959575.1 | QVFSELGLSYKAIGYAEFEAVKDKLVQVARLI |
| | | | | GQPVPSADAIYYKVPWEEVPELVAGRRVLLH |
| | | | | KGYAYIAIYQVVSLVATQFRSYLSKALSLTNR |
| | | | | KWTSTIREQEKDRLTPIVEALCTSYLGPDYSQP |
| | | | | TEYADISIKDLEQIAKSSFPLCMRHLFDKLKED |
| | | | | HHLKHGGRMQLGLFLKGVGLKLDDALAFWR |
| | | | | AEFSQRVGVERFDKEYAYSIRHNYGKEGKRV |
| | | | | DYSPYSCQKVISSSPGVGDHHGCPYRHFSEDN |
| | | | | LRAALGKMGVNNQKMEDILDKVRNRHYQLA |
| | | | | CTLTFESVHGSSCDAGINHPNQYFIDSQKVLQS |
| | | | | KNNSTA |
| Cucurbita | PriL | 4 | https://www. | ATGGAACTCCATCGTTCTCAGAGGAAATCTT |
| moschata | isoform | | ncbi.nlm. | CCACCGCGACCATTTCCTCGACCACTCTTCC |
| cv. Rifu | 2 CDS | | nih.gov/ | ACTCTATCGCTCCGCTCCTCCTCTCGAAGTC |
| | | | nuccore/XM_ | AGGCTTGAAGAATTCGAACTTTATGCCATAG |
| | | | 023103807.1 | ATCGTCTTCGAGTTCTTAAAGGAATTTCTGA |
| | | | | TGGTTTATCTCGAGGAAAGAAATCTGAAGA |
| | | | | CATGGAGAAACTGGTTAGAGACTTGTTGAA |
| | | | | GGCCCATATGAAACATCCACAGGCATCCGA |
| | | | | GGCTGTGAACAAGGATATAATATCTCACTTT |
| | | | | GTTCTGCGCCTCGTATACTGCAGAACGGAGG |
| | | | | ACTTGAGAAAATGGTTTCTTTCTATGGAAAC |
| | | | | TATGCTATTTCGACACCGTTTTCTTTCTGAAA |
| | | | | GTCCTGAATCTCAGAAGCAGGTCTTTTCGGA |
| | | | | GCTTGGCCTCTCATACAAAGCAATCGGTTAT |
| | | | | GCAGAGTTTGAGGCTGTGAAGGACAAATTG |
| | | | | GTCCAAGTTGCTCGGTTGATTGGTCAGCCTG |
| | | | | TACCAAGCGCTGATGCAATATACTATAAGGT |
| | | | | ACCATGGGAAGAAGTTCCAGAACTGGTGGC |
| | | | | TGGTCGAAGGGTATTACTTCATAAAGGATAT |
| | | | | GCATATATTGCTATCTATCAGGTGGTTTCCC |
| | | | | TTGTTGCAACACAATTCCGCAGTTACCTATC |
| | | | | AAAGGCCTTAAGTCTGACAAACAGGAAATG |
| | | | | GACATCTACAATAAGAGAACAAGAGAAGGA |
| | | | | TCGGTTGACCCCTATCGTTGAAGCCCTTTGC |
| | | | | ACTAGCTACCTGGGTCCTGACTACTCACAGC |
| | | | | CAACAGAGTATGCTGATATATCAATCAAAG |
| | | | | ACCTTGAACAAATTGCTAAAAGTTCATTTCC |
| | | | | TCTTTGCATGCGGCACCTATTTGATAAGCTG |
| | | | | AAAGAAGATCATCATTTGAAGCATGGAGGG |
| | | | | AGAATGCAATTAGGTCTCTTTCTTAAGGGTG |
| | | | | TTGGTTTGAAGCTTGATGATGCCCTTGCTTTC |
| | | | | TGGAGAGCTGAGTTCTCGCAGAGAGTTGGT |
| | | | | GTTGAGAGGTTTGACAAAGAATATGCATAC |
| | | | | AGTATCAGGCATAACTATGGAAAAGAAGGC |
| | | | | AAGAGAGTGGATTATTCACCTTATTCCTGTC |
| | | | | AAAAAGTCATCTCATCATCACCTGGTGTTGG |
| | | | | AGATCATCATGGATGTCCCTATAGACATTTC |
| | | | | AGTGAAGACAACTTAAGAGCAGCTCTTGGT |
| | | | | AAAATGGGAGTAAATAACCAGAAAATGGAA |
| | | | | GATATATTGGACAAAGTGCGAAATAGACAC |
| | | | | TATCAGTTGGCCTGCACCTTGACATTTGAAT |
| | | | | CGGTTCATGGTTCGTCATGCGACGCCGGGAT |
| | | | | TAATCATCCAAACCAGTACTTCATTGATAGT |
| | | | | CAAAAAGTTTTGCAATCTAAGAACAATTCAA |
| | | | | CAGCTTAG |
| Cucurbita | Modified | 5 | Not | MELHRSQRKSSTATTSSTTLPLYRSAPPLEVRL |
| moschata | PriL | | applicable | EEFELYAIDRLRVLKGISDGLSRGKKSEEMEKL |
| of the | Protein | | | VRDLLKAHMKHPQASEAVNKDIISHFVLRLVY |
| invention | | | | CRTEDLRKWFLSMETMLFRHRFLSKGPESQKQ |
| | | | | VFSELGLSYKAISYAECEAVKDKLVQVARLID |
| | | | | HPVPSADAIYYKVPWEEVPELVAGRRVLLHK |
| | | | | GYAYIAIYQVVSLVATQFRSYLSKALSLTNRK |
| | | | | WTSTIREQEKDRLTPIVEALCTSYLGPDYSQPT |
| | | | | EYADISIKDLEQIAKSSFPLCMRHLFDKLKEDH |
| | | | | HLKHGGRMQLGLFLKGVGLKLDDALAFWRA |
| | | | | EFSQRVGVERFDKEYAYSIRHNYGKEGKRVD |
| | | | | YSPYSCQKVISSSPGVGDHHGCPYRHFSEDNL |
| | | | | RAALGKMGVNNQKMEDILDKVRNRHYQLAC |
| | | | | TLTFESVHGSSCDAGINHPNQYFIDSQKVLQSK |
| | | | | NNSTA |
| Cucurbita | Modified | 6 | Not | ATGGAACTCCATCGTTCTCAGAGGAAATCTT |
| moschata | PriL | | applicable | CCACCGCCACCACTTCCTCGACCACTCTTCC |
| of the | CDS | | | ACTCTATCGCTCCGCTCCTCCTCTCGAAGTC |
| invention | | | | AGGCTTGAAGAATTCGAACTTTATGCCATAG |
| | | | | ATCGTCTTCGAGTTCTTAAAGGAATTTCTGA |
| | | | | TGGTTTATCTCGAGGAAAGAAATCTGAAGA |
| | | | | AATGGAGAAACTGGTTAGAGACTTGTTGAA |
| | | | | GGCCCATATGAAACATCCACAGGCATCCGA |
| | | | | GGCTGTGAACAAGGATATAATATCTCACTTT |
| | | | | GTTCTGCGCCTCGTATACTGCAGAACGGAGG |
| | | | | ACTTGAGAAAATGGTTTCTTTCTATGGAAAC |
| | | | | TATGCTATTTCGACACCGTTTTCTTTCTAAAG |
| | | | | GTCCTGAATCTCAGAAGCAGGTCTTTTCGGA |
| | | | | GCTTGGCCTCTCATACAAAGCAATCAGTTAT |
| | | | | GCAGAGTGTGAGGCTGTGAAGGACAAATTG |
| | | | | GTCCAAGTTGCTCGATTGATTGATCACCCTG |
| | | | | TACCAAGCGCTGATGCAATATACTATAAGGT |
| | | | | ACCATGGGAAGAAGTTCCAGAACTGGTGGC |
| | | | | TGGTCGAAGGGTATTACTTCATAAAGGATAT |
| | | | | GCATATATTGCTATCTATCAGGTGGTTTCCC |
| | | | | TTGTTGCAACACAATTCCGCAGTTACCTATC |
| | | | | AAAGGCCTTAAGTCTGACAAACAGGAAATG |
| | | | | GACATCTACAATAAGAGAACAAGAGAAGGA |
| | | | | TCGGTTGACCCCTATCGTTGAAGCCCTTTGC |
| | | | | ACAAGCTACCTGGGTCCTGACTACTCACAGC |
| | | | | CAACGGAGTATGCTGATATATCAATCAAAG |
| | | | | ACCTTGAACAAATTGCTAAAAGTTCATTTCC |
| | | | | TCTTTGCATGCGGCACCTATTTGATAAGCTG |
| | | | | AAAGAAGATCATCATTTGAAGCATGGAGGG |
| | | | | AGAATGCAATTAGGTCTCTTTCTCAAGGGTG |
| | | | | TTGGTTTGAAGCTTGATGATGCCCTTGCTTTC |
| | | | | TGGAGAGCTGAGTTCTCGCAGAGAGTTGGT |
| | | | | GTTGAGAGGTTTGACAAAGAATATGCATAC |
| | | | | AGTATCAGGCATAACTATGGAAAAGAAGGC |
| | | | | AAGAGAGTGGATTATTCACCTTATTCCTGTC |
| | | | | AAAAAGTCATCTCATCATCACCTGGTGTTGG |
| | | | | AGATCATCATGGATGTCCCTATAGACATTTC |
| | | | | AGTGAAGACAACTTAAGAGCAGCTCTTGGT |
| | | | | AAAATGGGAGTAAATAACCAGAAAATGGAA |
| | | | | GATATATTGGACAAAGTGCGAAATAGACAtT |
| | | | | ATCAGTTGGCCTGCACCTTGACGTTTGAATC |
| | | | | GGTTCATGGTTCATCGTGCGACGCTGGGATT |
| | | | | AATCATCCAAACCAGTACTTCATTGATAGTC |
| | | | | AAAAAGTTCTGCAATCTAAGAACAATTCAA |
| | | | | CAGCTTAG |
| Cucumis | PriL | 7 | http:// | MEPYLPQRKSSVSTNSTTALPLYRSAPPLEVRL |
| melo cv. | Protein | | cucurbitgenomics. | EDFELYAIDRLRVLKGISDGLSRGKKSEEMEK |
| DHL92 | | | org/feature/ | LVRELLKTNMKHPQASEVVNKDIISHFVLRLV |
| v3.5.1 | | | gene/ | YCRTEDLRKWFLSMETMLFRHRFLSESPESQK |
| | | | MELO3C022319 | QVFAELGLSYKAISNAEFEAVRDKLVQVARLI |
| | | | | GQPAPSSDAIYYKVPWEEVPELVAGRRVFLHK |
| | | | | GYAYIAIYQVVSLVATQFRSYLSKALSLTNRK |
| | | | | WTSTIREQEKDRLTPIVEALCTSYLGPDYSQPR |
| | | | | EYADISIKDLDQIAKSSFPLCMRHLFEKLKEDH |
| | | | | HLKHGGRMQLGLFLKGVGLKLDDALAFWRA |
| | | | | EFSQRVGAERFDKEYAYSIRHNYGKEGKRVD |
| | | | | YSPYSCQKIISSSPSVGDHHGCPYRHFSEDNLR |
| | | | | AALGKMGVNNRTMDDIMDKVRNRHYQVLNL |
| | | | | LPLNFYVVSWE |
| Cucumis | PriL | 8 | http:// | ATGGAACCCTATCTTCCTCAGAGAAAATCTT |
| melo cv. | CDS | | cucurbitgenomics. | CCGTCTCGACCAATTCCACTACCGCTCTCCC |
| DHL92 | | | org/feature/ | ACTCTACCGCTCTGCTCCTCCTCTTGAAGTC |
| v3.5.1 | | | gene/ | AGGCTTGAAGATTTTGAGCTTTATGCCATAG |
| | | | MELO3C022319 | ATCGTCTTCGAGTTCTTAAAGGGATTTCTGA |
| | | | | TGGGCTATCTCGAGGAAAGAAATCTGAAGA |
| | | | | AATGGAGAAACTGGTTAGAGAATTGTTGAA |
| | | | | AACCAACATGAAACATCCCCAGGCATCTGA |
| | | | | GGTTGTGAACAAGGATATAATATCTCACTTT |
| | | | | GTTCTGCGCCTTGTGTATTGCCGAACGGAGG |
| | | | | ACTTGAGAAAATGGTTTCTTTCTATGGAAAC |
| | | | | TATGCTATTCCGACACCGTTTTCTTTCTGAAA |
| | | | | GTCCTGAATCTCAGAAGCAGGTATTTGCGGA |
| | | | | GCTTGGTCTCTCATACAAAGCAATCAGTAAT |
| | | | | GCAGAATTTGAGGCTGTAAGGGACAAATTG |
| | | | | GTTCAAGTTGCTCGGTTGATTGGTCAGCCTG |
| | | | | CACCAAGTAGTGATGCTATATACTATAAGGT |
| | | | | ACCATGGGAAGAAGTTCCAGAGCTTGTGGC |
| | | | | TGGTCGAAGAGTATTTCTCCATAAAGGATAT |
| | | | | GCATATATTGCTATTTATCAGGTGGTTTCCCT |
| | | | | TGTTGCAACACAATTCCGCAGTTACCTGTCA |
| | | | | AAGGCCCTAAGTCTGACGAACAGGAAATGG |
| | | | | ACATCTACAATAAGAGAACAAGAGAAAGAT |
| | | | | CGGTTGACCCCAATAGTAGAAGCCCTTTGCA |
| | | | | CGAGCTACCTGGGTCCTGACTACTCACAGCC |
| | | | | AAGAGAGTATGCTGATATATCAATAAAAGA |
| | | | | CCTTGACCAAATAGCTAAAAGTTCATTTCCT |
| | | | | CTTTGCATGCGACACCTATTTGAAAAGCTGA |
| | | | | AAGAAGATCATCATTTGAAGCATGGAGGGA |
| | | | | GGATGCAATTAGGTCTCTTTCTCAAGGGTGT |
| | | | | TGGTTTGAAGCTTGATGATGCTCTGGCTTTC |
| | | | | TGGAGAGCTGAGTTCTCCCAGAGAGTTGGTG |
| | | | | CTGAGAGGTTTGACAAAGAATATGCATACA |
| | | | | GTATCAGGCATAACTATGGAAAAGAAGGCA |
| | | | | AGAGAGTGGATTATTCGCCTTATTCCTGTCA |
| | | | | AAAAATAATCTCATCATCACCTAGTGTTGGA |
| | | | | GATCATCATGGATGTCCCTATAGACATTTCA |
| | | | | GTGAAGACAACTTAAGAGCAGCTCTTGGTA |
| | | | | AAATGGGAGTAAATAACCGGACAATGGATG |
| | | | | ATATAATGGACAAAGTGCGAAATAGACATT |
| | | | | ATCAGGTTCTCAATTTACTGCCTCTTAATTTT |
| | | | | TATGTTGTCAGTTGGGAATAA |
| Cucumis | Modified | 9 | Not | MEPHLPQRKSSVSTNSTTALPLYRSAPPLEVRL |
| melo of | PriL | | applicable | EDFELYAIDRLRVLKGISDGLSRGKKSEEMEK |
| the | Protein | | | LVKELLKTNMKHPQASEVVNKDIISHFVLRLV |
| invention | | | | YCRTEDLRKWFLSMETMLFRHRFLSGSLESQK |
| | | | | QVFAELGLSYKAISNAEFEAVRDKLVQVARLI |
| | | | | GQPAQSSDAIYYKVPWEEVPELVAGRRVFLH |
| | | | | KGYAYIATYQVVSLVATQFRSYLSKALSLTNR |
| | | | | KWTSTIREQEKDRLTPIVEALCTSYLGPDYSQP |
| | | | | REYADISIKDLDQIAKSSFPLCMRHLFEKLKED |
| | | | | HHLKHGGRMQLGLFLKGVGLKLDDALAFWR |
| | | | | AEFSQRVGAERFDKEYAYSIRHNYGKEGKRV |
| | | | | DYSPYSCQKIISSSPSVGDHHGCPYRHFSEDNL |
| | | | | RAALGKMGVNNRTMDDIMDKVRNRHYQLAC |
| | | | | TLTFESIHGSTCDAGINHPNQYFIDSQKVLQSK |
| | | | | NNSTS |
| Cucumis | Modified | 10 | Not | ATGGAACCCCATCTTCCTCAGAGAAAATCTT |
| melo of | PriL | | applicable | CCGTCTCGACCAATTCCACTACCGCTCTCCC |
| the | CDS | | | ACTCTACCGCTCTGCTCCTCCTCTTGAAGTC |
| invention | | | | AGGCTTGAAGATTTTGAGCTTTATGCCATAG |
| | | | | ATCGTCTTCGAGTTCTTAAAGGGATTTCTGA |
| | | | | TGGGCTATCTCGAGGAAAGAAATCTGAAGA |
| | | | | AATGGAGAAACTGGTTAAAGAATTGTTGAA |
| | | | | AACCAACATGAAACATCCCCAGGCATCTGA |
| | | | | GGTTGTGAACAAGGATATAATATCTCACTTT |
| | | | | GTTCTGCGCCTTGTGTATTGCCGAACGGAGG |
| | | | | ACTTGAGAAAATGGTTTCTTTCTATGGAAAC |
| | | | | TATGCTATTCCGACACCGTTTTCTTTCTGGAA |
| | | | | GTCTTGAATCTCAGAAGCAGGTATTTGCGGA |
| | | | | GCTTGGTCTCTCATACAAAGCAATCAGTAAT |
| | | | | GCAGAATTTGAGGCTGTAAGGGACAAATTG |
| | | | | GTTCAAGTTGCTCGGTTGATTGGTCAGCCTG |
| | | | | CACAAAGTAGTGATGCTATATACTATAAGGT |
| | | | | ACCATGGGAAGAAGTTCCAGAGCTTGTGGC |
| | | | | TGGTCGAAGAGTATTTCTCCATAAAGGATAT |
| | | | | GCATATATTGCTACTTATCAGGTGGTTTCCC |
| | | | | TTGTTGCAACACAATTCCGCAGTTACCTGTC |
| | | | | AAAGGCCCTAAGTCTGACGAACAGGAAATG |
| | | | | GACATCTACAATAAGAGAACAAGAGAAAGA |
| | | | | TCGGTTGACCCCAATAGTAGAAGCCCTTTGC |
| | | | | ACGAGCTACCTGGGTCCTGACTACTCACAGC |
| | | | | CAAGAGAGTATGCTGATATATCAATAAAAG |
| | | | | ACCTTGACCAAATAGCTAAAAGTTCATTTCC |
| | | | | TCTTTGCATGCGACACCTATTTGAAAAGCTG |
| | | | | AAAGAAGATCATCATTTGAAGCATGGAGGG |
| | | | | AGGATGCAATTAGGTCTCTTTCTCAAGGGTG |
| | | | | TTGGTTTGAAGCTTGATGATGCTCTGGCTTT |
| | | | | CTGGAGAGCTGAGTTCTCCCAGAGAGTTGGT |
| | | | | GCTGAGAGGTTTGACAAAGAATATGCATAC |
| | | | | AGTATCAGGCATAACTATGGAAAAGAAGGC |
| | | | | AAGAGAGTGGATTATTCGCCTTATTCCTGTC |
| | | | | AAAAAATAATCTCATCATCACCTAGTGTTGG |
| | | | | AGATCATCATGGATGTCCCTATAGACATTTC |
| | | | | AGTGAAGACAACTTAAGAGCAGCTCTTGGT |
| | | | | AAAATGGGAGTAAATAACCGGACAATGGAT |
| | | | | GATATAATGGACAAAGTGCGAAATAGACAT |
| | | | | TATCAGTTGGCATGCACCTTGACATTTGAAT |
| | | | | CGATCCATGGCTCGACATGTGATGCTGGGAT |
| | | | | TAATCATCCAAACCAGTACTTCATTGATAGT |
| | | | | CAAAAGGTCCTGCAATCTAAGAATAATTCA |
| | | | | ACATCCTAG |
| Cucurbita | PriL | 11 | http:// | MELHRSQRKSSTATTSSTTLPLYRSAPPLEVRL |
| pepo subsp. | Protein | | cucurbitgenomics. | EEFELYAIDRLRVLKGISDGLSRGKKSEEMEKL |
| pepo | | | org/feature/ | VRDLLKAHMKHPQASEAVNKDIISHFVLRLVY |
| | | | gene/Cp4. | CRTEDLRKWFLSMETMLFRHRFLSESPESQKQ |
| | | | 1LG17g10680 | VFSELGLSYKAISYAEFEAVKDKLVQVARLIG |
| | | | | QPVPSADAIYYKVVSLVATQFRSYLSKALSLT |
| | | | | NRKWTSTIREQEKDRLTPIVEALCTSYLGPDYS |
| | | | | QPTEYADISIKDLEQIAKSSFPLCMRHLFDKLK |
| | | | | EDHHLKHGGRMQLGLFLKGVGLKLDDALAF |
| | | | | WRAEFSQRVGVERFDKEYAYSIRHNYGKEGK |
| | | | | RVLACTLTFESVHGSSCDAGINHPNQYFIDSQK |
| | | | | VLQSKHWENALARRVGSNQVLSLDREL |
| Cucurbita | PriL | 12 | http:// | ATGGAACTCCATCGTTCTCAGAGGAAATCTT |
| pepo subsp. | CDS | | cucurbitgenomics. | CCACCGCCACCACTTCCTCGACCACTCTTCC |
| pepo | | | org/feature/ | ACTCTATCGCTCAGCTCCTCCTCTCGAAGTC |
| | | | gene/Cp4. | AGGCTTGAAGAATTCGAACTTTATGCCATAG |
| | | | 1LG17g10680 | ATCGTCTTCGAGTTCTTAAAGGAATTTCTGA |
| | | | http:// | TGGTTTATCTCGAGGAAAAAAATCTGAAGA |
| | | | | AATGGAGAAACTGGTAAGAGACTTGTTGAA |
| | | | | GGCCCATATGAAACATCCACAGGCATCCGA |
| | | | | GGCTGTGAACAAGGATATAATATCTCACTTT |
| | | | | GTTCTGCGCCTCGTATACTGCAGAACGGAGG |
| | | | | ACTTGAGAAAATGGTTTCTTTCTATGGAAAC |
| | | | | TATGCTATTTCGACACCGTTTTCTTTCTGAAA |
| | | | | GTCCTGAATCTCAGAAGCAGGTCTTTTCGGA |
| | | | | GCTTGGCCTCTCATACAAAGCAATCAGTTAT |
| | | | | GCAGAGTTTGAGGCTGTGAAGGACAAATTG |
| | | | | GTCCAAGTTGCTCGGTTGATTGGTCAGCCTG |
| | | | | TACCAAGCGCTGATGCAATATACTATAAGGT |
| | | | | GGTTTCCCTTGTTGCAACACAATTCCGCAGT |
| | | | | TACCTATCAAAGGCCTTAAGTCTGACAAACA |
| | | | | GGAAATGGACATCTACAATAAGAGAACAAG |
| | | | | AGAAGGATCGGTTGACCCCTATCGTTGAAGC |
| | | | | CCTTTGCACAAGCTACCTGGGTCCTGACTAC |
| | | | | TCACAGCCAACAGAGTATGCTGATATATCAA |
| | | | | TCAAAGACCTTGAACAAATTGCTAAAAGTTC |
| | | | | ATTTCCTCTTTGCATGCGGCACCTATTTGATA |
| | | | | AGCTGAAAGAAGATCATCATTTGAAGCATG |
| | | | | GAGGGAGAATGCAATTAGGTCTCTTTCTCAA |
| | | | | GGGTGTTGGTTTGAAGCTTGATGATGCCCTT |
| | | | | GCTTTCTGGAGAGCTGAGTTCTCGCAGAGAG |
| | | | | TTGGTGTTGAGAGGTTTGACAAAGAATATGC |
| | | | | ATACAGTATCAGGCATAACTATGGAAAAGA |
| | | | | AGGCAAGAGAGTGTTGGCATGCACCTTGAC |
| | | | | GTTTGAATCGGTTCATGGTTCATCGTGCGAC |
| | | | | GCTGGGATTAATCATCCAAACCAGTACTTCA |
| | | | | TTGATAGTCAAAAAGTTCTGCAATCTAAGCA |
| | | | | CTGGGAAAACGCTCTTGCAAGGCGAGTGGG |
| | | | | TTCAAACCAGGTGCTTTCTCTCGATCGAGAG |
| | | | | CTTTAA |
| Cucurbita | PriL | 13 | https://www. | MELHRSQRKSSTATTSSTTLPLYRSAPPLEVRL |
| maxima cv. | Protein | | ncbi.nlm. | EEFELYAIDRLRVLKGISDGLSRGKKSEEMEKL |
| Rimu | | | nih.gov/ | VRGLLNAHMKHPQASEAVNKDIISHFVLRLVY |
| | | | protein/XP_ | CRTEDLRKWFLSMETMLFRHRFLSESPESQKQ |
| | | | 023004217.1 | VFSELGLSYKAISYAEFEAVKDKLVQVARLIG |
| | | | | QPVPSADAIYYKVPWEEVPELVAGRRVLLHK |
| | | | | GYAYIAIYQVVSLVATQFRSYLSKTLSLTNRK |
| | | | | WTSTIREQEKDRLTPIVEALCTSYLGPDYSQPT |
| | | | | EYADISIKDLEQIAKSSFPLCMRHLFDKLKEDH |
| | | | | HLKHGGRMQLGLFLKGVGLKLDDALTFWRA |
| | | | | EFSQRVGVERFDKEYAYSIRHNYGKEGKRVD |
| | | | | YSPYSCQKIISSSPGVGDHHGCPYRHFSEDNLR |
| | | | | AALGKMGVNNQKIEDILDKVRNRHYQLACTL |
| | | | | TFESVHGSSCDAGINHPNQYFIDSQKVLQSKN |
| | | | | NSTA |
| Cucurbita | PriL | 14 | https://www. | ATGGAACTCCATCGTTCTCAGAGGAAATCTT |
| maxima cv. | CDS | | ncbi.nlm. | CCACCGCCACCACTTCCTCGACCACTCTTCC |
| Rimu | | | nih.gov/ | ACTCTATCGCTCCGCTCCTCCTCTCGAAGTC |
| | | | nuccore/XM_ | AGGCTTGAAGAATTCGAACTTTATGCCATAG |
| | | | 023148449.1 | ATCGTCTTCGAGTTCTTAAAGGAATTTCTGA |
| | | | | TGGTTTATCTCGAGGAAAGAAATCTGAAGA |
| | | | | AATGGAGAAACTGGTTAGAGGCTTGTTGAA |
| | | | | TGCCCATATGAAACATCCACAGGCATCCGA |
| | | | | GGCTGTGAACAAGGATATAATATCTCACTTT |
| | | | | GTTCTGCGCCTCGTATACTGCAGAACGGAGG |
| | | | | ACTTGAGAAAATGGTTTCTTTCTATGGAAAC |
| | | | | TATGCTCTTTCGACACCGTTTTCTTTCTGAAA |
| | | | | GTCCTGAATCTCAGAAGCAGGTCTTTTCGGA |
| | | | | GCTTGGCCTCTCATACAAAGCAATCAGTTAT |
| | | | | GCAGAGTTTGAGGCTGTGAAGGACAAATTG |
| | | | | GTCCAAGTTGCTCGGTTGATTGGTCAGCCTG |
| | | | | TACCAAGCGCTGATGCAATATACTATAAGGT |
| | | | | ACCATGGGAAGAAGTTCCAGAACTGGTGGC |
| | | | | TGGTCGAAGGGTATTACTTCATAAAGGATAT |
| | | | | GCATATATTGCTATTTATCAGGTGGTTTCCCT |
| | | | | TGTTGCAACACAATTCCGCAGTTACCTATCA |
| | | | | AAGACCTTAAGTCTGACAAACAGGAAATGG |
| | | | | ACATCTACAATAAGAGAACAAGAGAAGGAT |
| | | | | CGGTTGACCCCTATCGTTGAAGCCCTTTGCA |
| | | | | CAAGCTACCTGGGTCCTGACTACTCACAGCC |
| | | | | AACAGAGTATGCTGATATATCAATCAAAGA |
| | | | | CCTTGAACAAATTGCTAAAAGTTCATTTCCT |
| | | | | CTTTGCATGCGGCACCTATTTGATAAGCTGA |
| | | | | AAGAAGATCATCATTTGAAGCATGGAGGGA |
| | | | | GAATGCAATTAGGTCTCTTTCTCAAGGGTGT |
| | | | | TGGTTTGAAGCTTGATGATGCCCTTACTTTCT |
| | | | | GGAGAGCTGAGTTCTCGCAGAGAGTTGGTG |
| | | | | TTGAGAGGTTTGACAAAGAATATGCATACA |
| | | | | GTATCAGGCATAACTATGGAAAAGAAGGCA |
| | | | | AGAGAGTGGATTATTCACCTTATTCCTGTCA |
| | | | | AAAAATTATCTCATCATCACCTGGTGTTGGA |
| | | | | GATCATCATGGATGTCCCTATAGACATTTCA |
| | | | | GTGAAGACAACTTAAGAGCAGCTCTAGGTA |
| | | | | AAATGGGAGTAAATAACCAGAAAATAGAAG |
| | | | | ATATATTGGACAAAGTGCGAAATAGACATT |
| | | | | ATCAGTTGGCCTGCACCTTGACATTTGAATC |
| | | | | GGTTCATGGTTCGTCGTGCGACGCTGGGATT |
| | | | | AATCATCCAAACCAGTACTTCATTGATAGTC |
| | | | | AAAAAGTTCTGCAATCTAAGAACAATTCAA |
| | | | | CAGCTTAG |
| Cucumis | PriL | 15 | http:// | MARFKLHLFSASVPRSVTAMEPYRSHRKSPIST |
| sativus L. | isoform | | cucurbitgenomics. | NSTTTLPLYRSAPPLEVRLEDFELYAIDRLRVL |
| var. sativus | 1 | | org.feature/ | KGISDGLSRGKKSEEMEKLVRELLKTNMKHP |
| cv. 9930 | Protein | | gene/Csa6G094750 | QASEVVNKDIISHFVLRLVYCRTEDLRKWFLS |
| (Chinese | | | | METMLFRHRFLSEGPESQKQVFAELGLSYKAI |
| Long) | | | | SNAEFEAVRDKLVQVARLIGQPAPSSDAIYYK |
| | | | | VPWEEVPELVAGRRVFLHKGYAYIAIYQVVSL |
| | | | | VATQFRSYLSKALSLTNRKWTSTIREQEKDRL |
| | | | | APIVEALCTSYLGPDYSQPREYADISIKDLDQIA |
| | | | | KSSFPLCMRHLFEKLKEDHHLKHGGRMQLGL |
| | | | | FLKGVGLKLDDALAFWRAEFSQRVGAERFDK |
| | | | | EYAYSIRHNYGKEGKRVDYSPYSCQKVISSSPS |
| | | | | VGDHHGCPYRHFSEDNLRAALGKMGVNNRT |
| | | | | MEDIMDKVRNRHYQLACTLTFESIHGSTCDAG |
| | | | | INHPNQYFIDSQKVLQSKNNSTS |
| Cucumis | PriL | 16 | http:// | ATGGCGCGTTTTAAACTCCATCTATTCTCAG |
| sativus L. | isoform | | cucurbitgenomics. | CTTCAGTGCCACGATCAGTGACTGCCATGGA |
| var. sativus | 1 CDS | | org/feature/ | ACCCTATCGTTCTCACAGAAAATCTCCCATC |
| cv. 9930 | | | gene/Csa6G094750 | TCAACCAATTCCACTACCACTCTCCCACTCT |
| (Chinese | | | | ACCGCTCTGCTCCTCCTCTTGAAGTCAGGCT |
| Long) | | | | TGAAGATTTTGAGCTTTATGCCATAGATCGT |
| | | | | 50CTTCGAGTTCTTAAAGGGATTTCTGATGGGT |
| | | | | TATCTCGAGGAAAGAAATCTGAAGAAATGG |
| | | | | AGAAACTGGTTAGAGAATTGTTGAAAACCA |
| | | | | ACATGAAACATCCACAGGCATCTGAGGTTGT |
| | | | | GAACAAGGATATAATATCTCACTTTGTTCTG |
| | | | | CGCCTTGTGTATTGCCGAACGGAGGACTTGA |
| | | | | GAAAATGGTTTCTTTCTATGGAAACTATGCT |
| | | | | ATTCCGACATCGTTTTCTTTCTGAAGGTCCTG |
| | | | | AATCTCAGAAGCAGGTATTTGCGGAGCTTGG |
| | | | | TCTCTCATACAAAGCAATCAGTAATGCAGAA |
| | | | | TTTGAGGCTGTAAGGGACAAATTGGTTCAAG |
| | | | | TTGCTCGGTTGATTGGTCAGCCTGCACCAAG |
| | | | | TAGTGATGCTATATACTATAAGGTACCATGG |
| | | | | GAAGAAGTTCCAGAGCTTGTGGCTGGTCGA |
| | | | | AGAGTATTCCTCCATAAAGGATATGCATATA |
| | | | | TTGCTATTTATCAGGTGGTTTCCCTTGTTGCA |
| | | | | ACACAATTCCGCAGTTACCTATCGAAGGCCC |
| | | | | TAAGTCTGACGAACAGGAAATGGACATCTA |
| | | | | CAATAAGAGAACAAGAGAAAGATCGGTTGG |
| | | | | CCCCAATAGTAGAAGCCCTTTGCACGAGCTA |
| | | | | CCTGGGTCCTGACTACTCACAGCCAAGAGA |
| | | | | GTATGCGGATATATCAATAAAGGACCTTGAC |
| | | | | CAAATAGCTAAAAGTTCATTTCCACTTTGCA |
| | | | | TGCGACACCTATTTGAAAAGCTGAAAGAAG |
| | | | | ATCATCATTTGAAGCATGGAGGGAGGATGC |
| | | | | AATTAGGTCTCTTTCTCAAGGGTGTTGGTTT |
| | | | | GAAGCTTGATGATGCCCTGGCTTTCTGGAGA |
| | | | | GCTGAGTTCTCCCAGAGAGTTGGTGCTGAGA |
| | | | | GGTTTGACAAAGAATATGCATACAGTATCA |
| | | | | GGCATAACTATGGAAAAGAAGGCAAGAGAG |
| | | | | TGGATTATTCGCCTTATTCCTGTCAAAAAGT |
| | | | | AATCTCATCATCACCTAGTGTTGGAGATCAT |
| | | | | CATGGATGTCCCTATAGACATTTCAGTGAAG |
| | | | | ACAACTTAAGAGCAGCTCTTGGTAAAATGG |
| | | | | GAGTAAATAACCGGACAATGGAAGATATAA |
| | | | | TGGACAAAGTGCGAAATAGACATTATCAGT |
| | | | | TGGCATGCACCTTGACATTTGAATCAATCCA |
| | | | | TGGCTCGACATGTGATGCTGGGATTAATCAT |
| | | | | CCAAACCAGTACTTCATTGATAGTCAAAAGG |
| | | | | TCCTGCAATCTAAGAATAATTCAACATCCTA |
| | | | | G |
| Cucumis | PriL | 17 | http:// | MARFKLHLFSASVPRSVTAMEPYRSHRKSPIST |
| sativus cv. | isoform | | cucurbitgenomics. | NSTTTLPLYRSAPPLEVRLEDFELYAIDRLRVL |
| Gy14 v2 | 2 | | org/feature/ | KGISDGLSRGKKSEEMEKLVRELLKTNMKHP |
| | Protein | | gene/CsGy6G007850 | QASEVVNKDIISHFVLRLVYCRTEDLRKWFLS |
| | | | | METMLFRHRFLSEGPESQVFAELGLSYKAISN |
| | | | | AEFEAVRDKLVQVARLIGQPAPSSDAIYYKVP |
| | | | | WEEVPELVAGRRVFLHKGYAYIAIYQVVSLV |
| | | | | ATQFRSYLSKALSLTNRKWTSTIREQEKDRLA |
| | | | | PIVEALCTSYLGPDYSQPREYADISIKDLDQIAK |
| | | | | SSFPLCMRHLFEKLKEDHHLKHGGRMQLGLF |
| | | | | LKGVGLKLDDALAFWRAEFSQRVGAERFDKE |
| | | | | YAYSIRHNYGKEGKRVDYSPYSCQKVISSSPS |
| | | | | VGDHHGCPYRHFSEDNLRAALGKMGVNNRT |
| | | | | MEDIMDKVRNRHYQLACTLTFESIHGSTCDAG |
| | | | | INHPNQYFIDSQKVLQSKFGTF |
| Cucumis | PriL | 18 | http:// | ATGGCGCGTTTTAAACTCCATCTATTCTCAG |
| sativus cv. | isoform | | cucurbitgenomics. | CTTCAGTGCCACGATCAGTGACTGCCATGGA |
| Gy14 v2 | 2 CDS | | org/feature/ | ACCCTATCGTTCTCACAGAAAATCTCCCATC |
| | | | gene/CsGy6G007850 | TCAACCAATTCCACTACCACTCTCCCACTCT |
| | | | | ACCGCTCTGCTCCTCCTCTTGAAGTCAGGCT |
| | | | | TGAAGATTTTGAGCTTTATGCCATAGATCGT |
| | | | | CTTCGAGTTCTTAAAGGGATTTCTGATGGGT |
| | | | | TATCTCGAGGAAAGAAATCTGAAGAAATGG |
| | | | | AGAAACTGGTTAGAGAATTGTTGAAAACCA |
| | | | | ACATGAAACATCCACAGGCATCTGAGGTTGT |
| | | | | GAACAAGGATATAATATCTCACTTTGTTCTG |
| | | | | CGCCTTGTGTATTGCCGAACGGAGGACTTGA |
| | | | | GAAAATGGTTTCTTTCTATGGAAACTATGCT |
| | | | | ATTCCGACATCGTTTTCTTTCTGAAGGTCCTG |
| | | | | AATCTCAGGTATTTGCGGAGCTTGGTCTCTC |
| | | | | ATACAAAGCAATCAGTAATGCAGAATTTGA |
| | | | | GGCTGTAAGGGACAAATTGGTTCAAGTTGCT |
| | | | | CGGTTGATTGGTCAGCCTGCACCAAGTAGTG |
| | | | | ATGCTATATACTATAAGGTACCATGGGAAG |
| | | | | AAGTTCCAGAGCTTGTGGCTGGTCGAAGAGT |
| | | | | ATTCCTCCATAAAGGATATGCATATATTGCT |
| | | | | ATTTATCAGGTGGTTTCCCTTGTTGCAACAC |
| | | | | AATTCCGCAGTTACCTATCGAAGGCCCTAAG |
| | | | | TCTGACGAACAGGAAATGGACATCTACAAT |
| | | | | AAGAGAACAAGAGAAAGATCGGTTGGCCCC |
| | | | | AATAGTAGAAGCCCTTTGCACGAGCTACCTG |
| | | | | GGTCCTGACTACTCACAGCCAAGAGAGTAT |
| | | | | GCGGATATATCAATAAAGGACCTTGACCAA |
| | | | | ATAGCTAAAAGTTCATTTCCACTTTGCATGC |
| | | | | GACACCTATTTGAAAAGCTGAAAGAAGATC |
| | | | | ATCATTTGAAGCATGGAGGGAGGATGCAAT |
| | | | | TAGGTCTCTTTCTCAAGGGTGTTGGTTTGAA |
| | | | | GCTTGATGATGCCCTGGCTTTCTGGAGAGCT |
| | | | | GAGTTCTCCCAGAGAGTTGGTGCTGAGAGGT |
| | | | | TTGACAAAGAATATGCATACAGTATCAGGC |
| | | | | ATAACTATGGAAAAGAAGGCAAGAGAGTGG |
| | | | | ATTATTCGCCTTATTCCTGTCAAAAAGTAAT |
| | | | | CTCATCATCACCTAGTGTTGGAGATCATCAT |
| | | | | GGATGTCCCTATAGACATTTCAGTGAAGACA |
| | | | | ACTTAAGAGCAGCTCTTGGTAAAATGGGAG |
| | | | | TAAATAACCGGACAATGGAAGATATAATGG |
| | | | | ACAAAGTGCGAAATAGACATTATCAGTTGG |
| | | | | CATGCACCTTGACATTTGAATCAATCCATGG |
| | | | | CTCGACATGTGATGCTGGGATTAATCATCCA |
| | | | | AACCAGTACTTCATTGATAGTCAAAAGGTCC |
| | | | | TGCAATCTAAGTTTGGAACTTTCTAA |
| Citrullus | PriL | 19 | http:// | MEPYRPQGKYSITTSSTTNLPLYRSAPPLEVKL |
| lanatus | isoform | | cucurbitgenomics. | EDFELYAIDRLRVLKGISDGLSRGKKSEEMEK |
| subsp. | 1 | | org/feature/ | LVRELWNTNMKHPQASEDVNKDIISHFVLRLV |
| vulgaris cv. | Protein | | gene/C1CG06G002100 | YCRTEELRKWFLSMETMLFRHRFLYASSESQK |
| Charleston | | | | QVFVELGLPYRAISYAEFEAVKDKLVQVARLI |
| Gray | | | | GQTVPSGAIYYKVVSLAATQFRSYLSKALSLT |
| | | | | NRKWTSTIREQEKDRLTPIVEALCTSYLGPDYS |
| | | | | QPREYGDISIKDLDQIAKSSFPLCMRHLFEKLK |
| | | | | EDHHLKHGGRMQLGLFLKGVGLKLDDALAF |
| | | | | WKAEFSQRVGAERFDKEYAYSIRHNYGKEGK |
| | | | | RVDYSPYSCQKIISSSPSVGDHHGCPYRHFSED |
| | | | | NLRAALGKMGVNNRTMDDIMDKVRNRHYQL |
| | | | | ACTLTFESIMSSCDAGINHPNQYFIDSQKVLQ |
| | | | | SKFTYQSLPTADSATHTRLLCLS |
| Citrullus | PriL | 20 | http:// | ATGGAACCCTATCGTCCTCAGGGAAAATATT |
| lanatus | isoform | | cucurbitgenomics. | CCATCACAACCAGTTCCACTACCAATCTTCC |
| subsp. | 1 CDS | | org/feature/ | TCTCTACCGCTCTGCTCCTCCTCTTGAAGTCA |
| vulgaris cv. | | | gene/C1CG06G002100 | AGCTCGAAGATTTCGAGCTTTATGCTATAGA |
| Charleston | | | | TCGTCTTCGAGTTCTTAAAGGGATTTCTGAT |
| Gray | | | | GGTTTATCTCGAGGAAAGAAATCTGAAGAA |
| | | | | ATGGAGAAACTGGTTAGAGAATTGTGGAAT |
| | | | | ACCAATATGAAACATCCACAGGCATCTGAG |
| | | | | GATGTGAACAAGGATATAATCTCTCACTTTG |
| | | | | TTTTGCGCCTTGTATACTGCAGAACGGAGGA |
| | | | | ATTGAGAAAATGGTTTCTTTCTATGGAAACT |
| | | | | ATGCTATTCCGACACCGTTTTCTTTATGCAA |
| | | | | GTTCTGAATCTCAGAAGCAGGTCTTTGTGGA |
| | | | | GCTTGGTCTCCCATACAGAGCAATCAGTTAT |
| | | | | GCAGAATTTGAGGCTGTAAAGGACAAATTG |
| | | | | GTTCAAGTTGCTCGGTTGATTGGTCAGACTG |
| | | | | TACCAAGTGGTGCTATATACTATAAGGTGGT |
| | | | | TTCCCTTGCTGCAACACAATTCCGCAGTTAC |
| | | | | CTATCAAAGGCCCTAAGTCTGACAAACAGG |
| | | | | AAATGGACATCTACAATAAGAGAACAAGAG |
| | | | | AAAGATAGGTTGACCCCAATAGTAGAAGCC |
| | | | | CTTTGCACGAGCTACCTGGGTCCTGACTACT |
| | | | | CACAGCCAAGAGAGTATGGTGATATATCAA |
| | | | | TAAAAGACCTTGACCAAATAGCTAAAAGTT |
| | | | | CATTTCCTCTTTGCATGCGACACCTATTTGA |
| | | | | AAAGCTGAAAGAAGATCATCATTTGAAGCA |
| | | | | TGGAGGGAGGATGCAATTAGGTCTCTTTCTC |
| | | | | AAGGGTGTTGGTTTGAAGCTTGATGATGCCC |
| | | | | TGGCTTTCTGGAAAGCTGAGTTCTCCCAGAG |
| | | | | AGTTGGTGCCGAGAGGTTTGACAAAGAATA |
| | | | | TGCATACAGTATCAGGCATAATTATGGAAA |
| | | | | AGAAGGCAAGAGAGTGGATTATTCGCCTTA |
| | | | | TTCCTGTCAAAAAATAATCTCATCATCACCT |
| | | | | AGTGTTGGAGATCATCATGGATGTCCCTACA |
| | | | | GACATTTCAGTGAAGACAACTTAAGAGCAG |
| | | | | CTCTTGGTAAAATGGGAGTAAATAACAGGA |
| | | | | CAATGGACGATATAATGGACAAAGTGCGAA |
| | | | | ATAGACATTATCAGTTGGCATGCACCTTGAC |
| | | | | ATTTGAATCGATCCATGGCTCGTCGTGTGAT |
| | | | | GCTGGGATTAATCATCCAAACCAGTACTTCA |
| | | | | TTGATAGTCAGAAGGTCCTTCAATCTAAGTT |
| | | | | TACATACCAAAGCCTACCTACTGCAGATTCG |
| | | | | GCTACACATACTCGGTTGTTGTGTCTCTCAT |
| | | | | AA |
| Citrullus | PriL | 21 | http:// | MEPYRPQGKYSITTSSTTNLPLYRSAPPLEVKL |
| lanatus | isoform | | cucurbitgenomics. | EDFELYAIDRLRVLKGISDGLSRGKKSEEMEK |
| subsp. | 2 | | org/feature/ | LVRELWNTNMKHPQASEDVNKDIISHFVLRLV |
| vulgaris cv. | Protein | | gene/C1a001518 | YCRTEELRKWFLSMETMLFRHRFLYASSESQK |
| 97103 | | | | QVFVELGLPYRAISYAEFEAVKDKLVQVARLI |
| | | | | GQTVPSGAIYYKVPWEEVPELVAGRRIFLHKG |
| | | | | YAYIAMYQVVSLAATQFRSYLSKALSLTNRK |
| | | | | WTSTIREQEKDRLTPIVEALCTSYLGPDYSQPR |
| | | | | EYGDISIKDLDQIAKSSFPLCMRHLFEKLKEDH |
| | | | | HLKHGGRMQLGLFLKGVGLKLDDALAFWKA |
| | | | | EFSQRVGAERFDKEYAYSIRHNYGKEGKRVD |
| | | | | YSPYSCQKIISSSPSVGDHHGCPYRHFSEDNLR |
| | | | | AALGKMGVNNRTMDDIMDKVRNRHYQLACT |
| | | | | LTFESIHGSSCDAGINHPNQYFIDSQKVLQSKV |
| | | | | EHCPH |
| Citrullus | PriL | 22 | http:// | ATGGAACCCTATCGTCCTCAGGGAAAATATT |
| lanatus | isoform | | cucurbitgenomics. | CCATCACAACCAGTTCCACTACCAATCTTCC |
| subsp. | 2 | | org/feature/ | TCTCTACCGCTCTGCTCCTCCTCTTGAAGTCA |
| vulgaris cv. | CDS | | gene/C1a001518 | AGCTCGAAGATTTCGAGCTTTATGCTATAGA |
| 97103 | | | | TCGTCTTCGAGTTCTTAAAGGGATTTCTGAT |
| | | | | GGTTTATCTCGAGGAAAGAAATCTGAAGAA |
| | | | | ATGGAGAAACTGGTTAGAGAATTGTGGAAT |
| | | | | ACCAATATGAAACATCCACAGGCATCTGAG |
| | | | | GATGTGAACAAGGATATAATCTCTCACTTTG |
| | | | | TTTTGCGCCTTGTATACTGCAGAACGGAGGA |
| | | | | ATTGAGAAAATGGTTTCTTTCTATGGAAACT |
| | | | | ATGCTATTCCGACACCGTTTTCTTTATGCAA |
| | | | | GTTCTGAATCTCAGAAGCAGGTCTTTGTGGA |
| | | | | GCTTGGTCTCCCATACAGAGCAATCAGTTAT |
| | | | | GCAGAATTTGAGGCTGTAAAGGACAAATTG |
| | | | | GTTCAAGTTGCTCGGTTGATTGGTCAGACTG |
| | | | | TACCAAGTGGTGCTATATACTATAAGGTACC |
| | | | | ATGGGAAGAAGTTCCAGAGCTAGTGGCTGG |
| | | | | TCGAAGAATATTTCTCCATAAAGGATATGCA |
| | | | | TATATTGCTATGTATCAGGTGGTTTCCCTTGC |
| | | | | TGCAACACAATTCCGCAGTTACCTATCAAAG |
| | | | | GCCCTAAGTCTGACAAACAGGAAATGGACA |
| | | | | TCTACAATAAGAGAACAAGAGAAAGATAGG |
| | | | | TTGACCCCAATAGTAGAAGCCCTTTGCACGA |
| | | | | GCTACCTGGGTCCTGACTACTCACAGCCAAG |
| | | | | AGAGTATGGTGATATATCAATAAAAGACCTT |
| | | | | GACCAAATAGCTAAAAGTTCATTTCCTCTTT |
| | | | | GCATGCGACACCTATTTGAAAAGCTGAAAG |
| | | | | AAGATCATCATTTGAAGCATGGAGGGAGGA |
| | | | | TGCAATTAGGTCTCTTTCTCAAGGGTGTTGG |
| | | | | TTTGAAGCTTGATGATGCCCTGGCTTTCTGG |
| | | | | AAAGCTGAGTTCTCCCAGAGAGTTGGTGCCG |
| | | | | AGAGGTTTGACAAAGAATATGCATACAGTA |
| | | | | TCAGGCATAATTATGGAAAAGAAGGCAAGA |
| | | | | GAGTGGATTATTCGCCTTATTCCTGTCAAAA |
| | | | | AATAATCTCATCATCACCTAGTGTTGGAGAT |
| | | | | CATCATGGATGTCCCTACAGACATTTCAGTG |
| | | | | AAGACAACTTAAGAGCAGCTCTTGGTAAAA |
| | | | | TGGGAGTAAATAACAGGACAATGGACGATA |
| | | | | TAATGGACAAAGTGCGAAATAGACATTATC |
| | | | | AGTTGGCATGCACCTTGACATTTGAATCGAT |
| | | | | CCATGGCTCGTCGTGTGATGCTGGGATTAAT |
| | | | | CATCCAAACCAGTACTTCATTGATAGTCAGA |
| | | | | AGGTCCTTCAATCTAAGGTAGAACATTGTCC |
| | | | | TCATTGA |
| Solanum | PriL | 23 | https://www. | MEAVRSQRKSLVSTGVGSTLPLYRSAPPLEVR |
| lycopersicum | Protein | | ncbi.nlm. | LEDFELYAIDRLRVLKGISDALSRGKKPDEME |
| cv. | | | nih.gov/ | KLVLDLWKTNMRHQHSSELLNKDIISHFVLRL |
| Heinz | | | protein/XP_ | VYCRTEELRKWFLSIETTLFRYRFRDEPPEKQR |
| 1706 | | | 004237566.1 | ALMADFDLPYKAVTIAEYESVKEKLNQVARAI |
| | | | | GQPITTDAIYYKVPFEEVPELVAGRRVFIQKGN |
| | | | | AYIAMNQVVSLVITQFRSHLSKALVLTNRKWT |
| | | | | SMIREQEKDRLVPIIEALSTSYLGPDYNQPREH |
| | | | | AEISLKDIDQIAKSSFPLCMGHLFEKLQEDHHL |
| | | | | KHGGRMQLGLFLKGVGLKLDDALAFWRAEFS |
| | | | | RKVGAERFEKEYAYSIRHNYGKEGKRTDYTP |
| | | | | YSCQKIISSTPGVGDHHGCPYRHFSEENLRAAL |
| | | | | TRMRVGNRALEDVIDKVRNRHYQLACTLTFE |
| | | | | AVHGSSCDAGVNHPNQYYNDSQRILESQKSSS |
| | | | | NPKGTAASM |
| Solanum | PriL | 24 | https://www. | ATGGAAGCGGTAAGATCGCAGCGAAAATCT |
| lycopersicum | CDS | | ncbi.nlm. | TTGGTATCGACCGGCGTCGGTTCAACACTTC |
| cv. | | | nih.gov/ | CTCTCTATCGCTCTGCCCCTCCACTTGAAGTC |
| Heinz | | | nuccore/XM_ | CGTCTTGAAGATTTCGAGCTTTACGCCATTG |
| 1706 | | | 0042237518.4 | ATCGCCTCCGAGTTCTTAAAGGTATTTCAGA |
| | | | | TGCTTTGTCTAGAGGAAAGAAACCCGATGA |
| | | | | AATGGAGAAATTGGTGTTGGATTTGTGGAA |
| | | | | AACAAATATGAGGCATCAACATTCATCTGA |
| | | | | GCTCCTTAATAAGGACATAATTTCACATTTT |
| | | | | GTTTTGCGACTTGTTTATTGCAGGACGGAGG |
| | | | | AGTTAAGAAAATGGTTTCTTTCAATTGAAAC |
| | | | | TACCTTATTTCGTTACCGTTTCCGCGATGAA |
| | | | | CCTCCTGAAAAACAGAGAGCGCTAATGGCA |
| | | | | GATTTTGATCTTCCATACAAAGCTGTAACCA |
| | | | | TTGCTGAATACGAGAGTGTAAAGGAAAAAT |
| | | | | TGAACCAAGTTGCACGCGCCATAGGCCAAC |
| | | | | CTATTACAACTGATGCTATCTATTACAAGGT |
| | | | | TCCATTTGAGGAGGTGCCAGAGCTTGTGGCA |
| | | | | GGTCGACGAGTATTTATTCAAAAAGGGAAT |
| | | | | GCATATATCGCCATGAATCAGGTGGTTTCAC |
| | | | | TAGTAATCACACAGTTTCGGAGTCATCTTTC |
| | | | | GAAAGCACTAGTGCTGACAAACAGAAAATG |
| | | | | GACCTCTATGATCAGAGAACAGGAGAAGGA |
| | | | | CCGTTTGGTTCCTATTATTGAAGCCTTATCCA |
| | | | | CAAGTTACCTGGGTCCTGATTATAACCAGCC |
| | | | | GAGAGAACATGCAGAAATATCACTAAAAGA |
| | | | | CATTGACCAGATTGCTAAGAGTTCATTCCCT |
| | | | | CTATGTATGGGTCATCTTTTCGAAAAGCTAC |
| | | | | AAGAGGATCATCATCTGAAGCATGGAGGGA |
| | | | | GGATGCAACTTGGTCTATTTCTCAAGGGTGT |
| | | | | TGGATTGAAGTTGGATGATGCCCTTGCATTC |
| | | | | TGGAGAGCTGAGTTCTCCCGAAAAGTTGGTG |
| | | | | CTGAAAGATTTGAAAAAGAATATGCATACA |
| | | | | GCATAAGACACAACTATGGGAAAGAAGGAA |
| | | | | AGAGAACGGATTACACACCTTATTCTTGTCA |
| | | | | AAAGATTATATCATCAACTCCTGGAGTTGGA |
| | | | | GATCACCATGGCTGTCCATATCGTCATTTCA |
| | | | | GTGAGGAGAATCTGAGAGCTGCTCTGACCA |
| | | | | GGATGAGAGTAGGCAATCGAGCACTGGAGG |
| | | | | ATGTGATAGACAAAGTCCGAAATAGACATT |
| | | | | ACCAGTTGGCATGCACTTTGACCTTTGAAGC |
| | | | | TGTTCATGGCTCATCTTGTGATGCCGGGGTT |
| | | | | AACCATCCAAATCAGTACTACAATGACAGTC |
| | | | | AGAGGATCTTGGAATCACAGAAGAGTTCCA |
| | | | | GTAACCCAAAAGGAACAGCAGCTTCAATGT |
| | | | | AG |
| Solanum | PriL | 25 | Not | MEAVKSQRKPGVSNGVVSNLPLYLSAPPLEVR |
| melongena | Protein | | applicable | LEDFELYAIDRLRVLKGISDALSRGKKPDEMG |
| | | | | KLVLDLWETNMRHQHSSEVINKDIISHFVLRL |
| | | | | VYCRTEELRKWFLSIETTLFRYRFRDKGPEVQ |
| | | | | RALMAEFDLPYKAVSNAEYESVKDKLIQVAR |
| | | | | AIGQHIETNAIFYKVPFEEVPELVAGRRVFIQK |
| | | | | GKAYIAMNQVVSLVITKFRSHLSKALVLTNRK |
| | | | | WTSMIRVQEKDRLVPIIEALSTSYLGPDYSQPR |
| | | | | ENAEISLKDIDQIAKSSFPLCMRHLFEKLREDH |
| | | | | HLKHGGRMQLGLFLKGVGLKLDDALAFWRA |
| | | | | EFSRKVGAERFDKEYAYGIRHIYGKEGKRTVG |
| | | | | KKQKKWLLSIFFQFLCSSECPCDYTPYSCQKIIS |
| | | | | SAPGVGDHHGCPYRHFSEENLRAALTRMRVG |
| | | | | NRTLEDVIDKVRNRHYQLACTLTFEAVHSSSC |
| | | | | DAGVNHPNQYYNDSQKILESQNSHSKPQGTT |
| | | | | ASM |
| Solanum | PriL | 26 | Not | ATGGAAGCCGTAAAGTCTCAGAGGAAACCT |
| melongena | CDS | | applicable | GGGGTATCGAACGGCGTCGTTTCAAATCTCC |
| | | | | CTCTCTACCTCTCTGCCCCTCCTCTTGAAGTT |
| | | | | CGTCTTGAAGATTTTGAGCTTTACGCCATCG |
| | | | | ATCGCCTTCGAGTTCTTAAAGGAATTTCGGA |
| | | | | TGCTTTGTCTAGAGGAAAGAAGCCGGATGA |
| | | | | AATGGGGAAATTGGTGTTGGATCTGTGGGA |
| | | | | AACAAATATGAGGCATCAACATTCATCTGA |
| | | | | GGTTATTAATAAGGACATAATTTCACATTTC |
| | | | | GTCTTGAGACTTGTTTATTGCAGGACGGAGG |
| | | | | AGTTAAGAAAATGGTTTCTTTCTATTGAAAC |
| | | | | TACCTTATTTCGTTATCGTTTCCGGGATAAA |
| | | | | GGTCCTGAAGTTCAGAGGGCACTTATGGCA |
| | | | | GAGTTTGATCTTCCATACAAAGCTGTGAGCA |
| | | | | ATGCTGAGTATGAAAGTGTGAAGGACAAAT |
| | | | | TGATCCAAGTTGCACGCGCCATAGGCCAAC |
| | | | | ATATTGAAACTAATGCTATCTTTTACAAGGT |
| | | | | TCCATTTGAGGAGGTGCCAGAGCTTGTGGCA |
| | | | | GGTCGAAGAGTATTTATTCAGAAAGGGAAG |
| | | | | GCATATATTGCCATGAATCAGGTGGTTTCGC |
| | | | | TAGTTATCACAAAGTTCCGGAGTCATCTTTC |
| | | | | GAAAGCACTAGTGTTGACTAACAGAAAATG |
| | | | | GACCTCGATGATCAGAGTACAAGAGAAGGA |
| | | | | CCGTTTGGTTCCTATAATTGAAGCCTTATCC |
| | | | | ACAAGTTACCTGGGTCCTGATTATAGCCAGC |
| | | | | CAAGAGAAAATGCAGAAATATCACTTAAAG |
| | | | | ACATTGATCAGATTGCTAAGAGTTCATTCCC |
| | | | | TCTATGTATGCGTCATCTTTTTGAAAAGCTA |
| | | | | CGAGAGGATCATCATCTGAAGCATGGAGGG |
| | | | | AGGATGCAGCTTGGTCTATTTCTCAAGGGTG |
| | | | | TTGGATTGAAGTTGGATGATGCCCTTGCATT |
| | | | | CTGGAGAGCTGAGTTCTCCCGAAAAGTTGGT |
| | | | | GCTGAAAGATTTGACAAAGAATATGCATAT |
| | | | | GGCATACGACACATCTATGGGAAAGAAGGA |
| | | | | AAGAGAACGGTAGGAAAAAAGCAGAAGAA |
| | | | | ATGGCTTTTGTCCATATTCTTTCAATTTCTCT |
| | | | | GTTCCTCTGAATGCCCATGTGATTACACTCC |
| | | | | TTATTCATGTCAAAAGATTATATCATCAGCT |
| | | | | CCTGGAGTAGGAGATCACCATGGCTGTCCAT |
| | | | | ATCGTCATTTCAGTGAGGAGAACCTGAGAG |
| | | | | CTGCTCTGACCAGGATGAGAGTAGGCAATC |
| | | | | GAACACTGGAGGATGTCATAGACAAAGTCC |
| | | | | GAAATAGACATTACCAGTTGGCATGCACTTT |
| | | | | GACCTTTGAAGCTGTTCATAGCTCGTCTTGC |
| | | | | GATGCCGGGGTTAACCATCCAAATCAGTACT |
| | | | | ACAATGACAGTCAGAAGATCTTGGAATCAC |
| | | | | AGAATAGTCATAGCAAGCCACAAGGAACAA |
| | | | | CAGCTTCAATGTAG |
| Capsicum | PriL | 27 | https://www. | MEAVRSQRKSSFSNGGGGGVSTLPIYRSAPPLE |
| annuum | isoform | | ncbi.nlm. | VRLEDFELYAVDRLRVLKRISDGLSRGKKPDE |
| cv. Zunla- | 1 | | nih.gov/ | MEKLVLDLWKTNMRHEHSSEVVNKDIISHFV |
| 1 | Protein | | protein/XP_ | LRLVYCRTEELRKWFLSMETTLFRYRFRDESP |
| | | | 016542575.1 | EIQRALMAEFDLQYKAVSNAEYESVKDKLNQ |
| | | | | VARAIGQPITSTDTIFYKNLHRIWSIYIGCFGYS |
| | | | | YDQEIWIDWVPFEEVPELVAGRRVLIQKGNAY |
| | | | | IAMNQVVSLVITQFRSLLSKALVLTNRKWTSM |
| | | | | IREQEKDRLTPIVEALSTSYLGPDYSQPREHAE |
| | | | | LSLKDIDQIAKSSFPLCMRHLFDKLREDHHLK |
| | | | | HGGRMQLGLFLKGVGLKLDDALAFWRAEFSQ |
| | | | | KVGAERFDKEYAYGIRHNYGKEGKRTDYTPY |
| | | | | SCQKIISAAPGVGDHHGCPYRHFSEENLRAAL |
| | | | | TRMGVGNRAMEDVMDKVRNRHYQLACTLTF |
| | | | | EAVHNSTCDAGVNHPNQYFNDSQKIFESKKSS |
| | | | | SNPKGAAAST |
| Capsicum | PriL | 28 | https://www. | ATGGAAGCTGTACGATCTCAGAGAAAATCTT |
| annuum | isoform | | ncbi.nlm. | CGTTTTCCAATGGCGGCGGCGGCGGAGTTTC |
| cv. Zunla- | 1 CDS | | nih.gov/ | AACTCTACCTATCTATCGCTCTGCTCCTCCTC |
| 1 | | | nuccore/XM_ | TCGAAGTTCGTCTTGAAGATTTCGAGCTTTA |
| | | | 016687089.1 | CGCCGTCGATCGCCTCCGAGTTCTAAAAAGA |
| | | | | ATTTCGGATGGTTTGTCTAGAGGAAAGAAGC |
| | | | | CAGATGAAATGGAGAAATTGGTGTTGGATC |
| | | | | TATGGAAAACAAATATGAGGCATGAACATT |
| | | | | CATCTGAGGTTGTTAATAAGGACATAATTTC |
| | | | | ACATTTTGTCTTGCGACTTGTTTATTGCAGG |
| | | | | ACGGAGGAGTTAAGAAAATGGTTTCTTTCAA |
| | | | | TGGAAACTACCTTATTTCGTTACCGTTTCCG |
| | | | | GGATGAGTCTCCTGAAATTCAGAGGGCGCT |
| | | | | AATGGCAGAGTTTGATCTTCAATACAAAGCT |
| | | | | GTGAGCAATGCTGAATATGAGAGTGTGAAG |
| | | | | GACAAATTGAATCAAGTTGCACGCGCTATA |
| | | | | GGCCAGCCTATTACAAGCACTGATACTATCT |
| | | | | TCTACAAGAATCTGCATCGGATTTGGTCTAT |
| | | | | TTATATTGGATGCTTTGGCTACAGCTATGAC |
| | | | | CAAGAAATATGGATTGACTGGGTTCCATTCG |
| | | | | AGGAGGTGCCAGAGCTTGTGGCTGGTCGAC |
| | | | | GAGTATTAATTCAGAAAGGGAATGCATATA |
| | | | | TTGCCATGAATCAGGTGGTTTCACTAGTTAT |
| | | | | CACACAGTTCCGAAGTCTTCTTTCCAAAGCA |
| | | | | CTGGTGCTGACAAACAGAAAATGGACTTCG |
| | | | | ATGATCAGAGAACAGGAGAAGGACCGTTTG |
| | | | | ACTCCTATTGTTGAAGCCTTATCCACAAGTT |
| | | | | ACCTGGGTCCTGATTATAGCCAGCCAAGAG |
| | | | | AACATGCAGAACTATCACTAAAAGACATTG |
| | | | | ATCAGATTGCTAAGAGTTCATTCCCTCTGTG |
| | | | | TATGCGTCATCTTTTCGATAAGCTACGTGAG |
| | | | | GATCATCATCTGAAACACGGAGGGAGGATG |
| | | | | CAACTTGGACTATTTCTCAAGGGTGTTGGAT |
| | | | | TGAAGTTGGATGATGCCCTTGCATTCTGGAG |
| | | | | AGCTGAGTTCTCCCAGAAAGTTGGTGCTGAA |
| | | | | AGATTTGATAAAGAATATGCATACGGCATA |
| | | | | AGACACAACTATGGGAAAGAAGGAAAGAG |
| | | | | AACGGACTACACACCTTATTCTTGTCAAAAG |
| | | | | ATTATATCGGCTGCTCCTGGAGTTGGAGATC |
| | | | | ACCATGGCTGTCCATATCGTCATTTCAGTGA |
| | | | | GGAGAATCTGAGAGCTGCTCTGACCAGGAT |
| | | | | GGGAGTAGGCAATCGAGCAATGGAGGATGT |
| | | | | GATGGACAAAGTCCGAAATAGACATTATCA |
| | | | | GTTGGCATGCACTTTGACCTTTGAAGCTGTT |
| | | | | CACAACTCAACTTGTGATGCGGGGGTTAACC |
| | | | | ATCCAAATCAGTACTTCAATGATAGTCAGAA |
| | | | | GATCTTTGAATCAAAGAAAAGTTCCAGCAA |
| | | | | CCCAAAAGGAGCAGCAGCGTCAACGTAG |
| Capsicum | PriL | 29 | | MEAVRSQRKSSFSNGGGGGVSTLPIYRSAPPLE |
| annuum | isoform | | | VRLEDFELYAVDRLRVLKRISDGLSRGKKPDE |
| cv. Zunla- | 2 | | | MEKLVLDLWKTNMRHEHSSEVVNKDIISHFV |
| 1 | Protein | | | LRLVYCRTEELRKWFLSMETTLFRYRFRDESP |
| | | | | EIQRALMAEFDLQYKAVSNAEYESVKDKLNQ |
| | | | | VARAIGQPITSTDTIFYKVPFEEVPELVAGRRV |
| | | | | LIQKGNAYIAMNQVVSLVITQFRSLLSKALVLT |
| | | | | NRKWTSMIREQEKDRLTPIVEALSTSYLGPDY |
| | | | | SQPREHAELSLKDIDQIAKSSFPLCMRHLFDKL |
| | | | | REDHHLKHGGRMQLGLFLKGVGLKLDDALAF |
| | | | | WRAEFSQKVGAERFDKEYAYGIRHNYGKEGK |
| | | | | RTDYTPYSCQKIISAAPGVGDHHGCPYRHFSEE |
| | | | | NLRAALTRMGVGNRAMEDVMDKVRNRHYQ |
| | | | | LACTLTFEAVHNSTCDAGVNHPNQYFNDSQKI |
| | | | | FESKKSSSNPKGAAAST |
| Capsicum | PriL | 30 | https://www. | ATGGAAGCTGTACGATCTCAGAGAAAATCTT |
| annuum | isoform | | ncbi.nlm. | CGTTTTCCAATGGCGGCGGCGGCGGAGTTTC |
| cv. Zunla- | 2 CDS | | nih.gov/ | AACTCTACCTATCTATCGCTCTGCTCCTCCTC |
| 1 | | | nuccore/XM_ | TCGAAGTTCGTCTTGAAGATTTCGAGCTTTA |
| | | | 016687090.1 | CGCCGTCGATCGCCTCCGAGTTCTAAAAAGA |
| | | | | ATTTCGGATGGTTTGTCTAGAGGAAAGAAGC |
| | | | | CAGATGAAATGGAGAAATTGGTGTTGGATC |
| | | | | TATGGAAAACAAATATGAGGCATGAACATT |
| | | | | CATCTGAGGTTGTTAATAAGGACATAATTTC |
| | | | | ACATTTTGTCTTGCGACTTGTTTATTGCAGG |
| | | | | ACGGAGGAGTTAAGAAAATGGTTTCTTTCAA |
| | | | | TGGAAACTACCTTATTTCGTTACCGTTTCCG |
| | | | | GGATGAGTCTCCTGAAATTCAGAGGGCGCT |
| | | | | AATGGCAGAGTTTGATCTTCAATACAAAGCT |
| | | | | GTGAGCAATGCTGAATATGAGAGTGTGAAG |
| | | | | GACAAATTGAATCAAGTTGCACGCGCTATA |
| | | | | GGCCAGCCTATTACAAGCACTGATACTATCT |
| | | | | TCTACAAGGTTCCATTCGAGGAGGTGCCAGA |
| | | | | GCTTGTGGCTGGTCGACGAGTATTAATTCAG |
| | | | | AAAGGGAATGCATATATTGCCATGAATCAG |
| | | | | GTGGTTTCACTAGTTATCACACAGTTCCGAA |
| | | | | GTCTTCTTTCCAAAGCACTGGTGCTGACAAA |
| | | | | CAGAAAATGGACTTCGATGATCAGAGAACA |
| | | | | GGAGAAGGACCGTTTGACTCCTATTGTTGAA |
| | | | | GCCTTATCCACAAGTTACCTGGGTCCTGATT |
| | | | | ATAGCCAGCCAAGAGAACATGCAGAACTAT |
| | | | | CACTAAAAGACATTGATCAGATTGCTAAGA |
| | | | | GTTCATTCCCTCTGTGTATGCGTCATCTTTTC |
| | | | | GATAAGCTACGTGAGGATCATCATCTGAAA |
| | | | | CACGGAGGGAGGATGCAACTTGGACTATTT |
| | | | | CTCAAGGGTGTTGGATTGAAGTTGGATGATG |
| | | | | CCCTTGCATTCTGGAGAGCTGAGTTCTCCCA |
| | | | | GAAAGTTGGTGCTGAAAGATTTGATAAAGA |
| | | | | ATATGCATACGGCATAAGACACAACTATGG |
| | | | | GAAAGAAGGAAAGAGAACGGACTACACACC |
| | | | | TTATTCTTGTCAAAAGATTATATCGGCTGCT |
| | | | | CCTGGAGTTGGAGATCACCATGGCTGTCCAT |
| | | | | ATCGTCATTTCAGTGAGGAGAATCTGAGAGC |
| | | | | TGCTCTGACCAGGATGGGAGTAGGCAATCG |
| | | | | AGCAATGGAGGATGTGATGGACAAAGTCCG |
| | | | | AAATAGACATTATCAGTTGGCATGCACTTTG |
| | | | | ACCTTTGAAGCTGTTCACAACTCAACTTGTG |
| | | | | ATGCGGGGGTTAACCATCCAAATCAGTACTT |
| | | | | CAATGATAGTCAGAAGATCTTTGAATCAAA |
| | | | | GAAAAGTTCCAGCAACCCAAAAGGAGCAGC |
| | | | | AGCGTCAACGTAG |
| Capsicum | PriL | 31 | https://www. | MEAVRSQRKSSFSNGGGGGVSTLPIYRSAPPLE |
| annuum | isoform | | ncbi.nlm. | VRLEDFELYAVDRLRVLKRISDGLSRGKKPDE |
| cv. Zunla- | 3 | | nih.gov/ | MEKLVLDLWKTNMRHEHSSEVVNKDIISHFV |
| 1 | Protein | | protein/XP_ | LRLVYCRTEELRKWFLSMETTLFRYRFRDESP |
| | | | 016542577.1 | EIQRALMAEFDLQYKAVSNAEYESVKDKLNQ |
| | | | | VARAIGQPITSTDTIFYKNLHRIWSIYIGCFGYS |
| | | | | YDQEIWIDWVPFEEVPELVAGRRVLIQKGNAY |
| | | | | IAMNQVVSLVITQFRSLLSKALVLTNRKWTSM |
| | | | | IREQEKDRLTPIVEALSTSYLGPDYSQPREHAE |
| | | | | LSLKDIDQIAKSSFPLCMRHLFDKLREDHHLK |
| | | | | HGGRMQLGLFLKGVGLKLDDALAFWRAEFSQ |
| | | | | KVGAERFDKEYAYGIRHNYGKEGKRTSAGLH |
| | | | | TLFLSKDYIGCSWSWRSPWLSISSFQ |
| Capsicum | PriL | 32 | https://www. | ATGGAAGCTGTACGATCTCAGAGAAAATCTT |
| annuum | isoform | | ncbi.nlm. | CGTTTTCCAATGGCGGCGGCGGCGGAGTTTC |
| cv. Zunla- | 3 CDS | | nih.gov/ | AACTCTACCTATCTATCGCTCTGCTCCTCCTC |
| 1 | | | nuccore/XM_ | TCGAAGTTCGTCTTGAAGATTTCGAGCTTTA |
| | | | 016687091.1 | CGCCGTCGATCGCCTCCGAGTTCTAAAAAGA |
| | | | | ATTTCGGATGGTTTGTCTAGAGGAAAGAAGC |
| | | | | CAGATGAAATGGAGAAATTGGTGTTGGATC |
| | | | | TATGGAAAACAAATATGAGGCATGAACATT |
| | | | | CATCTGAGGTTGTTAATAAGGACATAATTTC |
| | | | | ACATTTTGTCTTGCGACTTGTTTATTGCAGG |
| | | | | ACGGAGGAGTTAAGAAAATGGTTTCTTTCAA |
| | | | | TGGAAACTACCTTATTTCGTTACCGTTTCCG |
| | | | | GGATGAGTCTCCTGAAATTCAGAGGGCGCT |
| | | | | AATGGCAGAGTTTGATCTTCAATACAAAGCT |
| | | | | GTGAGCAATGCTGAATATGAGAGTGTGAAG |
| | | | | GACAAATTGAATCAAGTTGCACGCGCTATA |
| | | | | GGCCAGCCTATTACAAGCACTGATACTATCT |
| | | | | TCTACAAGAATCTGCATCGGATTTGGTCTAT |
| | | | | TTATATTGGATGCTTTGGCTACAGCTATGAC |
| | | | | CAAGAAATATGGATTGACTGGGTTCCATTCG |
| | | | | AGGAGGTGCCAGAGCTTGTGGCTGGTCGAC |
| | | | | GAGTATTAATTCAGAAAGGGAATGCATATA |
| | | | | TTGCCATGAATCAGGTGGTTTCACTAGTTAT |
| | | | | CACACAGTTCCGAAGTCTTCTTTCCAAAGCA |
| | | | | CTGGTGCTGACAAACAGAAAATGGACTTCG |
| | | | | ATGATCAGAGAACAGGAGAAGGACCGTTTG |
| | | | | ACTCCTATTGTTGAAGCCTTATCCACAAGTT |
| | | | | ACCTGGGTCCTGATTATAGCCAGCCAAGAG |
| | | | | AACATGCAGAACTATCACTAAAAGACATTG |
| | | | | ATCAGATTGCTAAGAGTTCATTCCCTCTGTG |
| | | | | TATGCGTCATCTTTTCGATAAGCTACGTGAG |
| | | | | GATCATCATCTGAAACACGGAGGGAGGATG |
| | | | | CAACTTGGACTATTTCTCAAGGGTGTTGGAT |
| | | | | TGAAGTTGGATGATGCCCTTGCATTCTGGAG |
| | | | | AGCTGAGTTCTCCCAGAAAGTTGGTGCTGAA |
| | | | | AGATTTGATAAAGAATATGCATACGGCATA |
| | | | | AGACACAACTATGGGAAAGAAGGAAAGAG |
| | | | | AACGAGTGCAGGACTACACACCTTATTCTTG |
| | | | | TCAAAAGATTATATCGGCTGCTCCTGGAGTT |
| | | | | GGAGATCACCATGGCTGTCCATATCGTCATT |
| | | | | TCAGTGA |

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### ToLCNDV Disease Testing

In the process of identifying new sources of ToLCNDV resistance, C. moschata 109238, C. pepo spp. pepo cv. 10006, and susceptible control C. pepo spp. pepo cv. Zucchini MU-CU-16 were subjected to a ToLCNDV disease test. Young plants of each of the genotypes were mechanically inoculated with a ToLCNDV isolate that was initially obtained from an infected field in Almeria, Spain, and multiplied in C. pepo plants. Mechanical inoculation of ToLCNDV was performed using the method adapted from Lopez et al. 2015, such that the ToLCNDV inoculum was prepared using buffer (i) as described (Euphytica. 2015 (204): 679-691). The ToLCNDV disease test was performed in a greenhouse with a daytime/night time temperature regime of 23° C./22° C. Five young plants of each genotype were mechanically inoculated twice, at 7 and 10 days after sowing. Two assessments were performed at approximately 17 and 24 days post sowing, by visual scoring for the amount of ToLCNDV symptoms, based on the scale described in Table 2. A plant having a disease score of 1-3 according to Table 2, is resistant to ToLCNDV. The disease test was performed in multiple years, between 2016 and 2019.

TABLE 2
ToLCNDV Plant Disease Test

| Disease | |
|---|---|
| Score | ToLCNDV symptoms on plants |
| 1 | No symptoms; healthy plant |
| 2 | Some non-specific yellowing due to aging, |
| | maturation or yellowing not |
| | related to viral infection |
| 3 | No leaf deformation, symptoms starting to |
| | develop; mainly on older leaves, |
| | some yellowing spots occur on less than |
| | 25% of the plant surface; re- |
| | growth and the top of the plant is symptomless |
| 4 | No leaf deformation, yellowing symptoms, |
| | 25-50% of the plant affected; |
| | yellow spots are more abundant than score 3; |
| | re-growth and the top of the plant is symptomless |
| 5 | No leaf deformation, severe yellowing symptoms; |
| | up to 100% of the plant affected with yellow |
| | spots and areas where yellow spots have merged in the |
| | larger yellow areas; symptoms are progressive |
| | even in newly formed leaves |
| 6 | Yellowing symptoms and some mild leaf deformation |
| | symptoms occur; some shoots and younger leaves |
| | show some deformed parts; some minor |
| | mottling in restricted areas |
| 7 | Severe yellowing; strong deformation and mottling in |
| | older leaves; in the younger parts, emerging shoots and |
| | newly formed leaves show some milder |
| | deformation; up to 75% of the plant surface shows |
| | deformation; plant is still growing |
| 8 | Severe leaf deformation, entire plant affected; |
| | the plant starts producing micro leaves and |
| | will no longer grow |
| 9 | Extreme severe leaf deformation, entire plant affected. |
| | Plants are dwarfed, necrotic or even die |

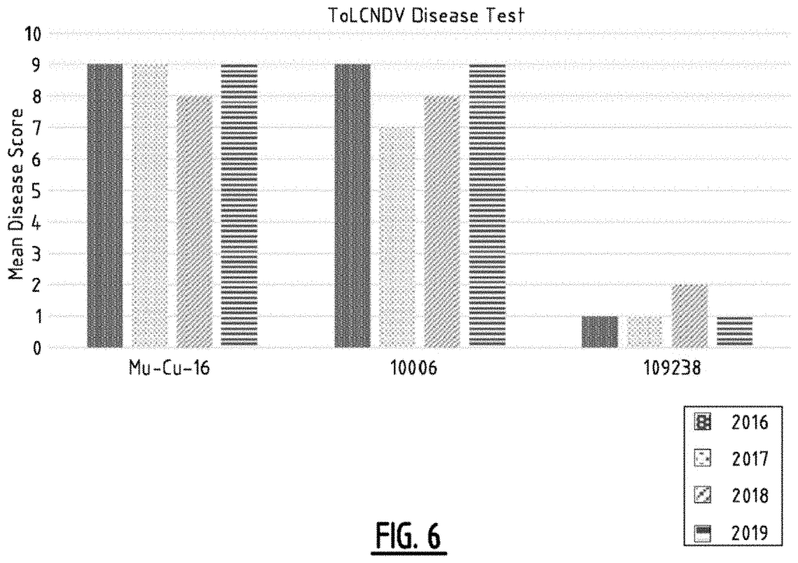

The mean disease score for each genotype was calculated for each of the years between 2016-2019 and is shown graphically in FIG. 6. It is clear from the results of the ToLCNDV Disease Test that C. moschata 109238 (labelled as ‘109238’ in FIG. 6) has a very high level of resistance against ToLCNDV, while C. pepo spp. pepo cv. 10006 (labelled as ‘10006’ in FIG. 6) and the susceptible control C. pepo spp. pepo cv. Zucchini MU-CU-16 (labelled as ‘Mu-Cu-16’ in FIG. 6) are susceptible to ToLCNDV.

The results of the mechanically inoculated ToLCNDV disease test, are confirmed in a ToLCNDV disease test in which whiteflies are used to transmit ToLCNDV to the plants, thereby mimicking the natural route of ToLCNDV infection.

### Example 2

### Identification of the Genomic Region and the Gene Responsible for ToLCNDV Resistance

An F2 mapping population comprising 173 individual F2 lines was developed using the C. moschata ToLCNDV resistant donor, 109238, obtained in Example 1 and C. pepo spp. pepo cv. 10006 (ToLCNDV susceptible), in order to map the genomic region responsible for ToLCNDV resistance. The 173 F2 lines as well as the parental lines, were phenotyped for ToLCNDV resistance using the disease test of Example 1, and genotyped using 43 markers.

A genetic map was constructed using an R software package, whereby markers that were non-polymorphic, had a strong segregation distortion or had an excess of missing data were removed. The genetic map was constructed in a two-step approach. First, the marker order was determined using the minimum spanning tree, after which this order was used as a starting order for regression mapping. The numbering and orientation of the linkage groups was determined using the publicly available C. pepo spp. pepo genome reference sequence available at: http://cucurbitgenomics.org/organism/14. Marker phase correction was performed using the marker information from the parental lines and the grouping structure.

QTL analysis was performed using an R software package. Outlier detection, normality assessment, and data transformation when considered necessary, was used to preprocess the data. Stepwise QTL analysis was then executed. Mapping of the data resulted in the identification of a quantitative trait loci (QTL) for ToLCNDV resistance of approximately 242 kB in size on chromosome 17.

Near isogenic lines (NILs) of BC1F2, BC2F2 and BC3F2 populations were developed from the parental lines of the F2 mapping population, using C. pepo spp. pepo cv. 10006 as the recurrent backcross (BC) parent. Multiple rounds of finemapping with additional markers, and phenotyping for ToLCNDV resistance using the disease test of Example 1, was performed on the NILs, in order to narrow down the QTL region and ultimately identify gene(s) responsible for the trait of the invention, namely ToLCNDV resistance. The QTL was narrowed down to a region of approximately 72 kB in size, which comprised one candidate gene in particular, the PriL gene.

The PriL gene of the donor C. moschata 109238 plant of the invention found to be resistant to ToLCNDV in the disease test of Example 1, was then sequenced. Sequencing revealed a modified PriL gene (SEQ ID NO: 6) in the resistant C. moschata donor plant of the invention, as compared to the sequence of the wild type PriL gene of the publicly available reference sequence, Cucurbita moschata cv. Rifu. This gene is mapped to chromosome 8 of the publicly available C. moschata cv. Rifu genome, and is syntenous to chromosome 17 of the publicly available C. pepo spp. pepo genome.

The CDS of the wild type C. moschata PriL gene isoform 1 and 2 are given in SEQ ID. No. 2 and SEQ ID. No. 4, respectively, and the protein sequence of the respective encoded wild type protein sequence is given in SEQ ID NO: 1 and SEQ ID NO: 3. Comparatively, the CDS sequence of the modified C. moschata PriL gene comprising the mutations listed in Table 3 and the protein sequence of the encoded modified PriL protein is given in SEQ ID NO: 6 and SEQ ID NO: 5, respectively.

A summary of the modifications to the PriL gene of the donor C. moschata plant of the invention, as well as the wild type PriL gene are listed in Table 3. The identified modifications result in amino acid substitutions to the wild type PriL protein sequence. Such modifications are not expected to severely change the native protein function of the PriL subunit but it is expected to have an effect on ToLCNDV resistance of the plant.

TABLE 3

| | Position | | | Position | | |
|---|---|---|---|---|---|---|
| | of the | | | of the | | |
| | SNP in | Wild | | mutation | | |
| Chromo- | the PriL | type | Mutant | in PriL | Amino | |
| some | gene | SNP | SNP | protein | acid | Type of |
| number¹ | CDS² (bp) | allele | allele | sequence³ | change | mutation |
| 8 | 440 | T | G | 147 | F > C | Amino acid |
| | | | | | | substitution |
| 8 | 485 | G | A | 162 | G > D | Amino acid |
| | | | | | | substitution |
| 8 | 489 | G | C | 163 | Q > H | Amino acid |
| | | | | | | substitution |

¹based on publicly available Cucurbita moschata cv. Rifu genome
²position based on Cucurbita moschata cv. Rifu, PriL isoform 1 (SEQ ID NO: 2) or isoform 2 (SEQ ID NO: 4) CDS sequence
³position based on Cucurbita moschata cv. Rifu, PriL isoform 1 (SEQ ID NO: 1) or isoform 2 (SEQ ID NO: 3) protein sequence

In the ToLCNDV resistant C. pepo seeds deposited under NCIMB accession number 43405, the modified PriL gene of the invention is homozygously present and comprises the 3 mutant SNP alleles outlined in Table 3. The CDS sequence of the modified PriL gene of the invention, comprising the 3 mutant SNP alleles (bolded and underlined) is shown in SEQ ID NO: 6. The sequence of the encoded modified PriL protein of the invention, comprising the 3 amino acid changes (bolded and underlined) is shown in SEQ ID NO: 5. These SNP sequences can be used to develop molecular markers for identifying ToLCNDV resistant C. pepo plants grown from said deposit.

### Example 3

### Identification of PriL Orthologs

Since ToLCNDV infection is a problem for members of the Cucurbitaceae and Solanaceae families, we focused on identifying the orthologous PriL gene in these species. Orthologs of the PriL gene were identified using a Basic Local Alignment Search Tool (BLAST) to compare the C. moschata PriL DNA and protein sequences with the genome of other Cucurbitaceae and Solanaceae species. Using this method, 1-2 best hits per species were identified as candidate PriL orthologous genes. CDS and protein sequences of the PriL orthologs that were identified through this method are shown in Table 1. Multiple sequence alignments (MSA) of the predicted protein sequences confirmed that these were orthologous PriL genes (FIG. 1). The wild type PriL protein of Cucumis melo, Cucurbita pepo, Cucurbita maxima, Cucumis sativus, Citrullus lanatus, Capsicum annuum, Solanum lycopersicum, and Solanum melongena have a high sequence identity and sequence similarity to the wild type C. moschata PriL protein (FIG. 2).

### Example 4

### Modified PriL gene in Cucumis melo Leads to ToLCNDV Resistance

In a screening for new sources of ToLCNDV resistance for C. melo, a donor source herein named C11R.10700-3, was identified using the same ToLCNDV disease test conditions and ToLCNDV isolate as outlined in Example 1, by visual scoring for the amount of ToLCNDV symptoms. Additionally, scoring was performed based on Table 4, which is a compact version of the scale in Table 2. A plant having a disease score of 0-1 according to Table 4, is resistant to ToLCNDV. Plants of melon cultivar Vedrantais known to be susceptible to ToLCNDV was used as a susceptible control in the disease test.

TABLE 4
ToLCNDV Plant Disease Test

| Disease | |
|---|---|
| Score | ToLCNDV symptoms on plants |
| 0 | No symptoms; healthy plant |
| 1 | No leaf deformation, symptoms starting to develop; |
| | mainly on older leaves, some yellowing |
| | spots occur on less than 25% of the plant surface; |
| | re-growth and the top of the plant is symptomless |
| 2 | No leaf deformation, severe yellowing symptoms; |
| | up to 100% of the plan taffected with yellow spots |
| | and areas where yellow spots have merged in |
| | the larger yellow areas; symptoms are progressive |
| | even in newly formed leaves |
| 3 | Severe yellowing; strong deformation and mottling |
| | in older leaves; in the younger parts, emerging shoots |
| | and newly formed leaves show some milder |
| | deformation; up to 75% of the plant surface shows |
| | deformation; plant is still growing |
| 4 | Extreme severe leaf deformation, entire plant affected. |
| | Plants are dwarfed, necrotic or even die |

Sequencing of the PriL gene of C11R.10700-3 revealed a modified PriL gene (SEQ ID NO: 10), as compared to the PriL gene sequence of Vedrantais. It is noted that the PriL gene of susceptible plants, such as Vedrantais, which were sequenced alongside the ToLCNDV resistant donor, had a gene sequence which was the same as the sequence of the PriL gene of the publicly available reference sequence, Cucumis melo cv. DHL92 v.3.5.1, which was characterized in the PriL ortholog gene identification of Example 3. Therefore, the CDS of the wild type C. melo PriL gene is given in SEQ ID. No. 8 and the encoded wild type protein sequence is given in SEQ ID NO: 7. Comparatively, the CDS sequence of the modified C. melo PriL gene of the invention comprising the mutations listed in Table 5 and the protein sequence of the encoded modified PriL protein is given in SEQ ID NO: 10 and SEQ ID NO: 9, respectively.

A summary of the modifications to the PriL gene of the invention, as well as the wild type PriL gene are listed in Table 5. The identified modifications result in amino acid substitutions to the wild type PriL protein sequence. Such modifications are not expected to severely change the native protein function of the PriL subunit but are expected to have an effect on ToLCNDV resistance of the plant.

TABLE 5

| | Position | | | Position | | |
|---|---|---|---|---|---|---|
| | of the | | | of the | | |
| | SNP in | Wild | | mutation | | |
| Chromo- | the PriL | type | Mutant | in PriL | Amino | |
| some | gene | SNP | SNP | protein | acid | Type of |
| number¹ | CDS² | allele | allele | sequence³ | change | mutation |
| 11 | 10 | T | C | 4 | Y > H | Amino acid |
| | | | | | | substitution |
| 11 | 497 | C | A | 166 | P > Q | Amino acid |
| | | | | | | substitution |
| 11 | 599 | T | C | 200 | I > T | Amino acid |
| | | | | | | substitution |

¹based on publicly available Cucumis melo cv. DHL92 v.3.5.1 genome
²position based on Cucumis melo cv. DHL92 v.3.5.1, PriL CDS sequence (SEQ ID NO: 8)
³position based on Cucumis melo cv. DHL92 v.3.5.1, PriL protein sequence (SEQ ID NO: 7)

Backcross (BC) families, developed from the ToLCNDV resistant donor C11R.10700-3 and two ToLCNDV susceptible C. melo lines as recurrent BC parents, were inbred to generate 51 segregating populations. Between 17 and 24 plants from each of these 51 populations were tested using the ToLCNDV disease test conditions and ToLCNDV isolate as outlined in Example 1. Scoring was performed based on Table 4. Additionally, polymorphic SNP markers were designed for the 3 wild type and mutant SNP alleles as outlined in Table 5, in order to genotype these populations. The genotyping and ToLCNDV disease test results are summarized in Table 6. Plants have been grouped based on the homo- or heterozygous presence or absence of the 3 wild type and mutant SNP alleles in the PriL gene.

The results in Table 6 illustrate that the modified PriL gene of the invention is recessively inherited and linked to the ToLCNDV resistance of the invention. Plants of the BC populations comprising in their genomes a PriL gene having the 3 mutant SNP alleles homozygously, of which they inherited from C11R.10700-3, are highly resistant to ToLCNDV. On the other hand, plants of the BC populations which comprise in their genomes a PriL gene having the 3 wild type SNP alleles homozygously, of which they inherited from their ToLCNDV susceptible BC parent, or the 3 SNP alleles heterozygously, are susceptible to ToLCNDV. For comparison, plants of the ToLCNDV susceptible control, Vedrantais, also comprise in their genome a PriL gene having the 3 wild type SNP alleles homozygously.

TABLE 6

| | | | | | | Average | |
|---|---|---|---|---|---|---|---|
| | | | | | Average | disease | |
| | Number | ¹SNP | ¹SNP | ¹SNP | disease | score at | |
| | of | Marker | Marker | Marker | score at 1^{st} | 2^{nd} | |
| Plants | plants | 1 | 2 | 3 | assessment | assessment | Phenotype |
| C11R.10700-3 | 42 | C/C | A/A | C/C | 0.09524 | 0.2381 | ToLCNDV |
| | | | | | | | Resistant |
| Plants of the | 160 | C/C | A/A | C/C | 0.15625 | 0.925 | ToLCNDV |
| BC | | | | | | | Resistant |
| populations | | | | | | | |
| comprising | | | | | | | |
| modified | | | | | | | |
| PriL gene | | | | | | | |
| Plants of the | 337 | T/C | C/A | T/C | 3.13947 | 3.35312 | ToLCNDV |
| BC | | | | | | | Susceptible |
| populations | | | | | | | |
| comprising | | | | | | | |
| heterozygous | | | | | | | |
| PriL gene | | | | | | | |
| Plants of the | 614 | T/T | C/C | T/T | 2.64658 | 3.05375 | ToLCNDV |
| BC | | | | | | | Susceptible |
| populations | | | | | | | |
| comprising | | | | | | | |
| wild type | | | | | | | |
| PriL gene | | | | | | | |
| Vedrantais | 42 | T/T | C/C | T/T | 4 | 4 | TolCNDV |
| | | | | | | | Susceptible |

¹SNP markers were developed based on position 10 (SNP marker 1), position 497 (SNP marker 2) and position 599 (SNP marker 3) of the C. melo PriL gene CDS according to SEQ ID NO: 8

To confirm the findings of the mechanically inoculated ToLCNDV disease test, plants were subjected to whitefly transmitted infection with ToLCNDV. Twelve plants per genotype of C11R.10700-3, C. melo plants comprising the modified and wild type alleles of the PriL gene and Vedrantais were sown. All plants used were thus at the same developmental age.

Half the plants of each genotype (i.e. six plants per genotype) were mechanically inoculated with ToLCNDV as described in Example 1. At 24 days after sowing, all plants, mechanically inoculated and uninfected plants, were transplanted into a greenhouse in a randomized block design. Approximately twenty non-viruliferous whiteflies (Bemisia tabaci) per plant were then released into the greenhouse in order to mimic natural infection. Two phenotypic assessments were performed at 5 and 7 weeks after infection on the whitefly transmitted ToLCNDV infected plants. Each plant was scored for the amount of ToLCNDV disease symptoms, based on the scale in Table 2, whereby a plant having a disease score of 1-3 according to Table 2, is resistant to ToLCNDV.

The average disease score at each assessment is shown in Table 7. As expected, the ToLCNDV resistant donor C11R.10700-3 and the C. melo plants comprising the modified PriL gene of the invention are highly resistant to ToLCNDV, while the susceptible control Vedrantais and C. melo plants comprising the wild type PriL gene are susceptible to ToLCNDV. Thus, the results of the whitefly transmitted ToLCNDV disease test confirmed the results obtained from the mechanically inoculated ToLCNDV disease test.

TABLE 7

| | Number of | | | | | | |
|---|---|---|---|---|---|---|---|
| | whitefly | | | | | Average | |
| | transmitted | | | | Average | disease | |
| | ToLCNDV | ¹SNP | ¹SNP | ¹SNP | disease | score at | |
| | infected | Marker | marker | marker | score at 1^{st} | 2^{nd} | |
| Plants | plants | 1 | 2 | 3 | assessment | assessment | Phenotype |
| C11R.10700-3 | 5 | C/C | A/A | C/C | 1 | 2.6 | ToLCNDV |
| | | | | | | | Resistant |
| Plants | 6 | C/C | A/A | C/C | 1 | 1 | ToLCNDV |
| comprising | | | | | | | Resistant |
| modified | | | | | | | |
| PriL gene | | | | | | | |
| Plants | 6 | T/T | C/C | T/T | 2.5 | 7 | ToLCNDV |
| comprising | | | | | | | Susceptible |
| wild type | | | | | | | |
| PriL gene | | | | | | | |
| Vedrantais | 6 | T/T | C/C | T/T | 2 | 6.2 | TolCNDV |
| | | | | | | | Susceptible |

¹SNP markers were developed based on position 10 (SNP marker 1), position 497 (SNP marker 2) and position 599 (SNP marker 3) of the C. melo PriL gene CDS according to SEQ ID NO: 8

In the ToLCNDV resistant C. melo seeds deposited under NCIMB accession number 43372, the modified PriL gene of the invention is homozygously present and comprises the 3 mutant SNP alleles outlined in Table 5. The CDS sequence of the modified PriL gene of the invention, comprising the 3 mutant SNP alleles (bolded and underlined) is shown in SEQ ID NO: 10. The sequence of the encoded modified PriL protein of the invention, comprising the 3 amino acid changes (bolded and underlined) is shown in SEQ ID NO: 9. These SNP sequences can be used to develop molecular markers for identifying ToLCNDV resistant C. melo plants grown from said deposit.

### Example 5

### Modifying the PriL Gene to Produce the ToLCNDV Resistance Trait

Seeds of the plant species of interest are mutagenized in order to introduce point mutations into the genome. Mutagenesis is achieved using chemical means, such as EMS treatment, or specific targeted means such as CRISPR. The skilled person is familiar with both chemical and targeted means for introducing mutations into a genome.

Mutagenized seed is then germinated, the resultant plants are selfed or crossed to produce M2 seed. A tilling screen for PriL gene modifications which are responsible for ToLCNDV resistance is performed. PriL gene modifications are identified based on comparison to the wild type PriL DNA sequences listed in SEQ ID NO: 2, SEQ ID NO: 4, SEQ ID NO: 8, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, or SEQ ID NO: 32 for the given plant species. The skilled person is also familiar with tilling (McCallum et. al. (2000) Nature Biotechnology, 18: 455-457) and techniques for identifying nucleotide changes such as DNA sequencing, amongst others.

Plants with a modified PriL gene are homozygous or made homozygous by selfing, crossing or doubled haploid techniques which are familiar to the skilled person. Plants identified and selected on the basis of modifications to the PriL gene, can then be tested in a ToLCNDV disease test to confirm that the ToLCNDV resistance results from one or more modifications of the PriL gene.

The invention is further described by the following numbered paragraphs:

1. A modified PriL gene, which encodes a modified protein comprising one or more modifications in the wild type protein sequence of SEQ ID NO: 1 or SEQ ID NO: 3 or in a protein sequence having at least 60% sequence identity to SEQ ID NO: 1 or SEQ ID NO: 3.

2. The modified PriL gene of paragraph 1, wherein the protein sequence encoded by the modified PriL gene has at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1 or SEQ ID NO: 3.

3. The modified PriL gene of paragraphs 1 or 2, the wild type of which encodes a protein according to SEQ ID NO: 1, SEQ ID NO: 3, SEQ ID NO: 7, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, or SEQ ID NO: 31.

4. The modified PriL gene of any one of the paragraphs 1 to 3, wherein the one or more modifications in the wild type protein sequence is an amino acid substitution.

5. The modified PriL gene of any one of the paragraphs 1 to 4, wherein the modified PriL gene encodes a modified protein that comprises a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5, and/or wherein the modified gene encodes a modified protein that comprises a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9.

6. The modified PriL gene of any one of the paragraphs 1 to 5, wherein the modified protein as a result of the one or more modifications, imparts Begomovirus resistance selected from ToLCNDV and/or ToLCPMV and/or CuLCV and/or MCLCV and/or MLCV and/or SqLCV and/or CuLCrV and/or TYLCV and/or WmCSV and/or WmCMoV resistance, in a plant in which the gene encoding the modified protein is homozygously present.

7. A modified PriL protein as defined in any one of the paragraphs 1 to 6.

8. The modified PriL protein of paragraph 7, wherein the modified protein as a result of the one or more modifications, imparts Begomovirus resistance selected from ToLCNDV and/or ToLCPMV and/or CuLCV and/or MCLCV and/or MLCV and/or SqLCV and/or CuLCrV and/or TYLCV and/or WmCSV and/or WmCMoV resistance in a plant, when the modified gene encoding the modified protein is homozygously present in the genome of the plant.

9. A plant, comprising the modified PriL gene of any one of the paragraphs 1 to 6.

10. The plant of paragraph 9, wherein the modified gene encoding the modified protein is homozygously present in the genome of the plant and the plant is resistant to Begomoviruses selected from the group consisting of ToLCNDV, ToLCPMV, CuLCV, MCLCV, MLCV, SqLCV, CuLCrV, TYLCV, WmCSV and WmCMoV.

11. A seed comprising the modified PriL gene of any one of the paragraphs 1 to 6, wherein the modified gene encoding the modified protein is homozygously present in the genome of the plant grown from said seed and the plant is resistant to Begomoviruses selected from the group consisting of ToLCNDV, ToLCPMV, CuLCV, MCLCV, MLCV, SqLCV, CuLCrV, TYLCV, WmCSV and WmCMoV.

12. A plant of paragraph 9 or 10, or a seed of paragraph 11, wherein the plant or seed belongs to any one of the species Cucurbita moschata, Cucurbita pepo, Cucumis melo, Cucumis sativus, Cucurbita maxima, Citrullus lanatus, Solanum lycopersicum, Solanum melongena or Capsicum annuum, in particular Cucurbita moschata, Cucurbita pepo or Cucumis melo.

13. A progeny plant of the plant of paragraph 9 , 10 or 12, or of the plant grown from the seed of paragraph 11 or 12, wherein the modified gene encoding the modified protein is homozygously present in the genome of the progeny plant and the progeny plant is resistant to Begomoviruses selected from the group consisting of ToLCNDV, ToLCPMV, CuLCV, MCLCV, MLCV, SqLCV, CuLCrV, TYLCV, WmCSV and WmCMoV.

14. A fruit harvested from the plant of paragraph 9, 10 or 12, or from a plant grown from the seed of paragraph 11 or 13, wherein the fruit comprises the modified PriL gene of any one of the paragraphs 1 to 6.

15. Propagation material suitable for producing the plant of paragraph 9, 10 or 12, wherein the propagation material is suitable for sexual reproduction, and is in particular selected from a microspore, pollen, ovary, ovule, embryo sac and egg cell, or is suitable for vegetative reproduction, and is in particular selected from a cutting, root, stem cell, and protoplast, or is suitable for tissue culture of regenerable cells or protoplasts, which regenerable cells or protoplasts are in particular selected from a leaf, pollen, embryo, cotyledon, hypocotyl, meristematic cell, root, root tip, anther, flower and stem, and wherein the propagation material comprises the modified PriL gene of any one of the paragraphs 1 to 6 that confers resistance to Begomoviruses selected from the group consisting of ToLCNDV, ToLCPMV, CuLCV, MCLCV, MLCV, SqLCV, CuLCrV, TYLCV, WmCSV and WmCMoV.

16. Use of a modified PriL gene of any one of the paragraphs 1 to 6 for producing a plant that is resistant to Begomoviruses selected from the group consisting of ToLCNDV, ToLCPMV, CuLCV, MCLCV, MLCV, SqLCV, CuLCrV, TYLCV, WmCSV and WmCMoV.

17. Use of the modified PriL gene of paragraph 16, wherein the resistant plant is a plant of the species Cucurbita moschata, Cucurbita pepo, Cucumis melo, Cucumis sativus, Cucurbita maxima, Citrullus lanatus, Solanum lycopersicum, Solanum melongena or Capsicum annuum, in particular Cucurbita moschata, Cucurbita pepo or Cucumis melo.

18. Use of paragraph 16 or 17, wherein the resistant plant is produced by mutagenesis or introgression, or combinations thereof.

19. A method for producing a Begomovirus resistant plant, said method comprising:

(a) crossing a plant comprising a modified PriL gene of any one of the paragraphs 1 to 6 with another plant to obtain an F1 population;

(b) optionally performing one or more rounds of selfing and/or crossing a plant from the F1 to obtain a further generation;

(c) selecting from the population a plant that comprises the modified PriL gene and is resistant to Begomoviruses selected from the group consisting of ToLCNDV, ToLCPMV, CuLCV, MCLCV, MLCV, SqLCV, CuLCrV, TYLCV, WmCSV and WmCMoV.

20. The method of paragraph 19, wherein the plant comprising the modified PriL gene in step a) is a plant of paragraph 9, 10 or 12 or is a plant grown from seed deposited under NCIMB accession number 43372 or 43405.

21. A method for producing a Begomovirus resistant plant, said method comprising:

(a) introducing one or more mutations in a population of plants;

(b) selecting a plant showing resistance to Begomoviruses selected from the group consisting of ToLCNDV, ToLCPMV, CuLCV, MCLCV, MLCV, SqLCV, CuLCrV, TYLCV, WmCSV and WmCMoV;

(c) verifying if the plant selected in step (b) has a mutation in its PriL gene, and selecting a plant comprising such a mutation;

(d) growing the plant obtained in step (c),

wherein the wild type PriL gene encodes a protein comprising at least 60% sequence identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1 or SEQ ID NO: 3.

22. A method for identifying a Begomovirus resistant plant, said method comprising:

(a) assaying genomic nucleic acids of a plant for the presence of one or more modifications in the PriL gene;

(b) identifying or selecting a plant as a plant that is resistant to Begomoviruses selected from the group consisting of ToLCNDV, ToLCPMV, CuLCV, MCLCV, MLCV, SqLCV, CuLCrV, TYLCV, WmCSV and WmCMoV, if one or more modifications in the PriL gene are present;

(c) optionally verifying if the plant is resistant to Begomoviruses selected from the group consisting of ToLCNDV, ToLCPMV, CuLCV, MCLCV, MLCV, SqLCV, CuLCrV, TYLCV, WmCSV and WmCMoV.

23. The method of paragraph 21 or 22, wherein the one or more modifications in the PriL gene is a SNP that results in a phenylalanine (F) to cysteine (C) substitution at a position corresponding to position 147 according to SEQ ID NO: 5, and/or a glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5, and/or wherein the one or more modifications in the PriL gene is a SNP that results in a tyrosine (Y) to histidine (H) substitution at a position corresponding to position 4 according to SEQ ID NO: 9, and/or proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9.

24. The method of any one of the paragraphs 18 to 22, wherein the plant is a plant of the species Cucurbita moschata, Cucurbita pepo, Cucumis melo, Cucumis sativus, Cucurbita maxima, Citrullus lanatus, Solanum lycopersicum, Solanum melongena or Capsicum annuum, in particular Cucurbita moschata, Cucurbita pepo or Cucumis melo.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. 1. A modified PriL gene, which encodes a modified protein comprising one or more modifications in the wild type protein sequence of SEQ ID NO: 1 or SEQ ID NO: 3 or in a protein sequence having at least 60% sequence identity to SEQ ID NO: 1 or SEQ ID NO: 3.

2. The modified PriL gene of claim 1, wherein the protein sequence encoded by the modified PriL gene has at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1 or SEQ ID NO: 3.

3. The modified PriL gene of claim 1, the wild type of which encodes a protein according to SEQ ID NO: 1, SEQ ID NO: 3, SEQ ID NO: 7, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, or SEQ ID NO: 31.

4. The modified PriL gene of claim 1, wherein the one or more modifications in the wild type protein sequence is an amino acid substitution.

5. The modified PriL gene of claim 1, wherein the modified PriL gene encodes a modified protein that comprises a phenylalanine (F) to cysteine (C) substitution at a position corresponding to position 147 according to SEQ ID NO: 5, and/or glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5, and/or wherein the modified gene encodes a modified protein that comprises a tyrosine (Y) to histidine (H) substitution at a position corresponding to position 4 according to SEQ ID NO: 9, and/or a proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9.

6. The modified PriL gene of claim 1, wherein the modified protein as a result of the one or more modifications, imparts Begomovirus resistance selected from ToLCNDV and/or ToLCPMV and/or CuLCV and/or MCLCV and/or MLCV and/or SqLCV and/or CuLCrV and/or TYLCV and/or WmCSV and/or WmCMoV resistance, in a plant in which the gene encoding the modified protein is homozygously present.

7. A modified PriL protein as defined in claim 1.

8. The modified PriL protein of claim 7, wherein the modified protein as a result of the one or more modifications, imparts Begomovirus resistance selected from ToLCNDV and/or ToLCPMV and/or CuLCV and/or MCLCV and/or MLCV and/or SqLCV and/or CuLCrV and/or TYLCV and/or WmCSV and/or WmCMoV resistance in a plant, when the modified gene encoding the modified protein is homozygously present in the genome of the plant.

9. A plant, comprising the modified PriL gene as claimed in claim 1.

10. The plant of claim 9, wherein the modified gene encoding the modified protein is homozygously present in the genome of the plant and the plant is resistant to Begomoviruses selected from the group consisting of ToLCNDV, ToLCPMV, CuLCV, MCLCV, MLCV, SqLCV, CuLCrV, TYLCV, WmCSV and WmCMoV.

11. A seed comprising the modified PriL gene of claim 1, wherein the modified gene encoding the modified protein is homozygously present in the genome of the plant grown from said seed and the plant is resistant to Begomoviruses selected from the group consisting of ToLCNDV, ToLCPMV, CuLCV, MCLCV, MLCV, SqLCV, CuLCrV, TYLCV, WmCSV and WmCMoV.

12. The plant of claim 9, wherein the plant belongs to any one of the species Cucurbita moschata, Cucurbita pepo, Cucumis melo, Cucumis sativus, Cucurbita maxima, Citrullus lanatus, Solanum lycopersicum, Solanum melongena or Capsicum annuum, in particular Cucurbita moschata, Cucurbita pepo or Cucumis melo.

13. The seed of claim 11, wherein the plant or seed belongs to any one of the species Cucurbita moschata, Cucurbita pepo, Cucumis melo, Cucumis sativus, Cucurbita maxima, Citrullus lanatus, Solanum lycopersicum, Solanum melongena or Capsicum annuum, in particular Cucurbita moschata, Cucurbita pepo or Cucumis melo.

14. A progeny plant of the plant of claim 9, wherein the modified gene encoding the modified protein is homozygously present in the genome of the progeny plant and the progeny plant is resistant to Begomoviruses selected from the group consisting of ToLCNDV, ToLCPMV, CuLCV, MCLCV, MLCV, SqLCV, CuLCrV, TYLCV, WmCSV and WmCMoV.

15. A progeny plant of the plant grown from the seed of claim 11, wherein the modified gene encoding the modified protein is homozygously present in the genome of the progeny plant and the progeny plant is resistant to Begomoviruses selected from the group consisting of ToLCNDV, ToLCPMV, CuLCV, MCLCV, MLCV, SqLCV, CuLCrV, TYLCV, WmCSV and WmCMoV.

16. A fruit harvested from the plant of claim 9, wherein the fruit comprises the modified PriL gene.

17. A fruit harvested from a plant grown from the seed of claim 11, wherein the fruit comprises the modified PriL gene.

18. A propagation material suitable for producing the plant of claim 9, wherein the propagation material is suitable for sexual reproduction, and is in particular selected from a microspore, pollen, ovary, ovule, embryo sac and egg cell, or is suitable for vegetative reproduction, and is in particular selected from a cutting, root, stem cell, and protoplast, or is suitable for tissue culture of regenerable cells or protoplasts, which regenerable cells or protoplasts are in particular selected from a leaf, pollen, embryo, cotyledon, hypocotyl, meristematic cell, root, root tip, anther, flower and stem, and wherein the propagation material comprises the modified PriL gene that confers resistance to Begomoviruses selected from the group consisting of ToLCNDV, ToLCPMV, CuLCV, MCLCV, MLCV, SqLCV, CuLCrV, TYLCV, WmCSV and WmCMoV.

19. A method for producing a Begomovirus resistant plant, said method comprising:
(a) crossing a plant comprising the modified PriL gene of claim 1 with another plant to obtain an F1 population;
(b) optionally performing one or more rounds of selfing and/or crossing a plant from the F1 to obtain a further generation;
(c) selecting from the population a plant that comprises the modified PriL gene and is resistant to Begomoviruses selected from the group consisting of ToLCNDV, ToLCPMV, CuLCV, MCLCV, MLCV, SqLCV, CuLCrV, TYLCV, WmCSV and WmCMoV.

20. The method of claim 19, wherein the plant comprising the modified PriL gene in step a) is the plant of claim 9 or is a plant grown from seed deposited under NCIMB accession number 43372 or 43405.

21. A method for producing a Begomovirus resistant plant, said method comprising:
(a) introducing one or more mutations in a population of plants;
(b) selecting a plant showing resistance to Begomoviruses selected from the group consisting of ToLCNDV, ToLCPMV, CuLCV, MCLCV, MLCV, SqLCV, CuLCrV, TYLCV, WmCSV and WmCMoV;
(c) verifying if the plant selected in step (b) has a mutation in its PriL gene, and selecting a plant comprising such a mutation;
(d) growing the plant obtained in step (c),
wherein the wild type PriL gene encodes a protein comprising at least 60% sequence identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1 or SEQ ID NO: 3.

22. A method for identifying a Begomovirus resistant plant, said method comprising:
(a) assaying genomic nucleic acids of a plant for the presence of one or more modifications in the PriL gene;
(b) identifying or selecting a plant as a plant that is resistant to Begomoviruses selected from the group consisting of ToLCNDV, ToLCPMV, CuLCV, MCLCV, MLCV, SqLCV, CuLCrV, TYLCV, WmCSV and WmCMoV, if one or more modifications in the PriL gene are present;
(c) optionally verifying if the plant is resistant to Begomoviruses selected from the group consisting of ToLCNDV, ToLCPMV, CuLCV, MCLCV, MLCV, SqLCV, CuLCrV, TYLCV, WmCSV and WmCMoV.

23. The method of claim 21 or 22, wherein the one or more modifications in the PriL gene is a SNP that results in a phenylalanine (F) to cysteine (C) substitution at a position corresponding to position 147 according to SEQ ID NO: 5, and/or a glycine (G) to aspartic acid (D) substitution at a position corresponding to position 162 according to SEQ ID NO: 5, and/or a glutamine (Q) to histidine (H) substitution at a position corresponding to position 163 according to SEQ ID NO: 5, and/or wherein the one or more modifications in the PriL gene is a SNP that results in a tyrosine (Y) to histidine (H) substitution at a position corresponding to position 4 according to SEQ ID NO: 9, and/or proline (P) to glutamine (Q) substitution at a position corresponding to position 166 according to SEQ ID NO: 9, and/or an isoleucine (I) to threonine (T) substitution at a position corresponding to position 200 according to SEQ ID NO: 9.

24. The method of claim 19, 21 or 22, wherein the plant is a plant of the species Cucurbita moschata, Cucurbita pepo, Cucumis melo, Cucumis sativus, Cucurbita maxima, Citrullus lanatus, Solanum lycopersicum, Solanum melongena or Capsicum annuum, in particular Cucurbita moschata, Cucurbita pepo or Cucumis melo.
